(19) United States
(12) Patent Application Publication
(10) Pub. No.: US 2023/0313283 A1
(43) Pub. Date: Oct. 5, 2023

(54) ISOTHERMAL NUCLEIC ACID DETECTION ASSAYS AND USES THEREOF

(71) Applicant: TRUSTEES OF BOSTON UNIVERSITY, Boston, MA (US)

(72) Inventors: Alexander GREEN, Chestnut Hill, MA (US); Zhaoqing YAN, Boston, MA (US); James ROBSON, Tyngsborough, MA (US); Padric Michael GARDEN, Boston, MA (US); Milad Babaei AMAMEH, Needham, MA (US)

(73) Assignee: TRUSTEES OF BOSTON UNIVERSITY, Boston, MA (US)

(21) Appl. No.: 18/115,335

(22) Filed: Feb. 28, 2023

Related U.S. Application Data
(60) Provisional application No. 63/314,743, filed on Feb. 28, 2022.

(51) Int. Cl. C12Q 1/6853; C12Q 1/6804

(52) U.S. Cl. CPC C12Q 1/6853; C12Q 1/6804; C12Q 2600/16

(57) ABSTRACT

The technology described herein is directed to methods, kits, and compositions for detecting a target nucleic acid, such as a viral RNA.

### CROSS-REFERENCE TO RELATED APPLICATIONS

This application claims benefit under § 119(e) of U.S. Provisional Application No. 63/314,743 filed Feb. 28, 2022, the contents of which is incorporated herein by reference in its entirety.

### GOVERNMENT SUPPORT

This invention was made with government support under AI148319 and EB031893 awarded by the National Institutes of Health. The government has certain rights in the invention.

### TECHNICAL FIELD

The technology described herein relates generally to methods, compositions, kits and systems for sensitive, specific, and reliable detection of target nucleic acids.

### BACKGROUND

Recent innovations in isothermal amplification of specific target nucleic acid sequences, paired with visual readout of the result have brought the prospect of highly sensitive point-of-care (POC) diagnostics that are fast, cheap, and use readily accessible equipment. For example, Loop-Mediated Isothermal Amplification (LAMP), recombinase polymerase amplification (RPA) and Helicase-dependent isothermal DNA amplification (HDA) are isothermal amplification methods that can be used to detect a target nucleic acid. However, amplicon detection in these assays can be limited by poor specificity or sensitivity. For example, LAMP is frequently used to test for the presence or absence of specific nucleic acid targets in a sample by coupling the amplification with a reporting scheme. A reporting scheme is an observable output, like a color change or fluorescence emission, that is only produced when the target is present, or that shows a distinguishable difference from the output produced when no target is present. The two most common reporting schemes for LAMP are colorimetric output and fluorescent output. In colorimetric output, the LAMP reaction is supplemented with a dye (e.g. phenol red) that changes color in response to a change in pH. Amplification of DNA results in a change in the pH of the solution, which is visualized by the naked eye or a machine as a color change. In fluorescent output, the LAMP reaction is supplemented with a conditionally fluorescent DNA binding dye. The fluorescence increases significantly in the presence of DNA amplicons, which is detected by a fluorescent reader. The drawbacks of these reporting techniques are two-fold. First, they are not sequence specific and hence any spurious amplification (to which all amplification schemes are prone) will result in a false positive. Second, they cannot produce distinct reporting based on the target sequence and hence cannot distinguish between multiple targets.

RPA-amplified DNA detection schemes with lateral flow device (LFD) readout rely on non-DNA signals such as fluorophores or biotin, initially on separate primers but brought together during amplification. These have intrinsically limited specificity, since RPA is error prone, and primer ‘dimers’ or other non-specific connections result in positive signals on LFD. There have been several demonstrations of the application of RPA products to LFDs for rapid visual detection of target amplicons, but they lack the capability of checking the target amplicon in a sequence specific way which would eliminate the problem of false positives from RPA background amplicons.

Thus, there is a great need for methods and kits for detecting target nucleic acids with minimal background during detection and that address one or more of the above noted issues in the field. The present disclosure addresses these needs.

### SUMMARY

The technology described herein relates, in general, to the identification of nucleic acid targets using compositions related to methods, compositions, kits and systems comprising a nucleic acid strand and a molecule capable of stabilizing or enhancing interactions between two nucleic acids for sensitive, specific, and reliable detection of target nucleic acids of interest.

In one aspect as described herein is a method for detecting a target nucleic acid, the method comprising: contacting a double-stranded or single-stranded amplicon from amplification of a target nucleic acid with a first probe to form a complex comprising the first probe and the amplicon, wherein the first probe comprises a first nucleic acid strand and a molecule bound with the first nucleic acid strand, wherein the molecule is capable of localizing a single-stranded nucleic acid to a double-stranded nucleic acid or enhancing the kinetics of hybridization between two single-stranded nucleic acids, and wherein the first nucleic acid strand comprises a binding domain comprising a nucleotide sequence substantially complementary to at least a first portion of the amplicon; and detecting the complex comprising the first probe and the amplicon.

In some embodiments of any of the aspects described herein, one of the amplicon and the first nucleic acid strand comprises a reporter molecule capable of producing a detectable signal and the other of the amplicon and the first nucleic acid strand comprises a capture ligand, and wherein said step of detecting the complex comprises detecting the reporter molecule in the complex.

In some embodiments of any of the aspects described herein, the first nucleic acid strand comprises a first hybridization domain linked with the binding domain linked with a second hybridization domain, and the first probe further comprises a second nucleic acid strand hybridized with the first nucleic acid strand, wherein the second nucleic acid strand comprises a first hybridization domain linked with a non-hybridizing domain linked with a second hybridization domain, wherein the first hybridization domain of the first nucleic acid strand and the second hybridization domain of the second nucleic acid strand are hybridized with each other forming a first double-stranded region, wherein the second hybridization domain of the first nucleic acid strand and the first hybridization domain of the second nucleic acid strand are hybridized with each other forming a second double-stranded region, wherein the binding domain of the first nucleic acid strand and the non-hybridizing domain of the second nucleic acid strand do not hybridize to each other, and wherein one of the amplicon and the first nucleic acid strand comprises a reporter molecule capable of producing a detectable signal and the other of the amplicon and the first nucleic acid strand comprises a capture ligand, and wherein said step of detecting the complex comprises detecting the reporter molecule in the complex.

In some embodiments of any of the aspects described herein, the first probe comprises a second nucleic acid strand hybridized with the first strand and forming a double-stranded structure comprising a single-stranded loop region, wherein the first nucleic acid strand comprises a first hybridization domain linked to the binding domain linked to a second hybridization domain, wherein the second nucleic acid strand comprises a first hybridization domain linked to a non-hybridizing domain linked to a second hybridization domain, wherein the first hybridization domain of the first nucleic acid strand and the second hybridization domain of the second nucleic acid strand are hybridized with each other forming a first double-stranded region, wherein the second hybridization domain of the first nucleic acid strand and the first hybridization domain of the second nucleic acid strand are hybridized with each other forming a second double-stranded region, wherein the binding domain of the first nucleic acid strand and the non-hybridizing domain of the second nucleic acid strand do not hybridize to each other, wherein the first nucleic acid strand comprises a reporter molecule capable of producing a detectable signal and the second nucleic acid strand comprises a capture ligand, and wherein absence of a detectable signal from the reporter molecule indicates presence of the target nucleic acid.

In some embodiments of any of the aspects described herein, the method comprises contacting the amplicon with the first probe and a second probe to form a complex comprising the first probe, the second probe and the amplicon, and wherein the second probe comprises a first nucleic acid strand (first nucleic acid of the second probe) and a molecule bound with the first nucleic acid strand of the second probe and capable of localizing a single-strand nucleic acid strand to a double-stranded nucleic acid or enhancing the kinetics of hybridization between two single-stranded nucleic acids, and wherein the first nucleic acid strand of the second probes comprises a binding domain comprising a nucleotide sequence substantially complementary to at least a second portion of the amplicon, wherein one of the first nucleic acid strand of the first probe and the first nucleic acid strand of the second probe comprises a reporter molecule capable of producing a detectable signal and the other of the first nucleic acid strand of the first probe and the first nucleic acid strand of the second probe comprises a capture ligand, and wherein said step of detecting the complex comprises detecting the reporter molecule in the complex.

In some embodiments of any of the aspects described herein, the first nucleic acid strand of the first probe comprises a reporter molecule capable of producing a detectable signal, and wherein said step of detecting the complex comprises: contacting the complex with a lateral flow device or a micro-array plate, wherein the lateral flow device or the micro-array plate comprises a capture/test region comprising a capture nucleic acid strand immobilized thereon, wherein the capture nucleic acid strand is bound with a molecule capable of localizing a single-strand nucleic acid strand to a double-stranded nucleic acid or enhancing the kinetics of hybridization between two single-stranded nucleic acids, and wherein the capture nucleic acid strand comprises a nucleotide sequence substantially complementary to at least a second portion of the amplicon; and detecting the reporter molecule in the complex captured by the capture probe.

In some embodiments of any of the aspects described herein, the first nucleic acid strand comprises a first hybridization domain linked to the binding domain, and the first probe comprises a second nucleic acid strand hybridized with the hybridization domain of the first strand, wherein one of the first and second nucleic strand comprises a reporter molecule capable of producing a detectable signal and the other of the first and second nucleic strand comprises a quencher molecule, and wherein the quencher molecule quenches the detectable signal from the reporter molecule when the first and second nucleic acid strands are hybridized with each other, wherein the first hybridization domain and the binding domain together comprise a nucleotide sequence substantially complementary to at least a portion of the amplicon, and wherein said step of detecting the complex comprises detecting a detectable signal produced by the reporter molecule.

In some embodiments of any of the aspects described herein, the first nucleic acid strand comprises a first hybridization domain linked with the binding domain linked with a second hybridization domain, and the first probe comprises a second nucleic acid strand hybridized with the first nucleic acid strand, wherein the second nucleic acid strand comprises a first hybridization domain linked with a non-hybridizing domain linked with a second hybridization domain, wherein the first hybridization domain of the first nucleic acid strand and the second hybridization domain of the second nucleic acid strand are hybridized with each other forming a first double-stranded region, wherein the second hybridization domain of the first nucleic acid strand and the first hybridization domain of the second nucleic acid strand are hybridized with each other forming a second double-stranded region, wherein the binding domain of the first nucleic acid strand and the non-hybridizing domain of the second nucleic acid strand do not hybridize to each other, wherein one of the first and second nucleic strand comprises a reporter molecule capable of producing a detectable signal and the other of the first and second nucleic strand comprises a quencher molecule, and wherein the quencher molecule quenches the detectable signal from the reporter molecule when the first and second nucleic acid strands are hybridized with each other, and wherein said step of detecting the complex comprises detecting a detectable signal produced by the reporter molecule.

In some embodiments of any of the aspects described herein, the first nucleic acid strand comprises a first hybridization domain linked to the binding domain, and the first probe further comprises a second nucleic acid strand hybridized with the hybridization domain of the first strand, wherein the first or second nucleic strand comprises a capture ligand, and wherein the amplicon comprises a reporter molecule capable of producing a detectable signal, and wherein said step of detecting the complex comprises: contacting the complex with a lateral flow device or micro-array plate, wherein the lateral flow device or the micro-array plate comprises a capture/test region comprising a capture probe immobilized thereon, wherein the capture probe is capable of binding with capture ligand; and detecting the reporter molecule in a complex captured by the capture probe.

In some embodiments of any of the aspects described herein, the first nucleic acid strand comprises the capture ligand, and the amplicon comprises the reporter molecule, and wherein the capture ligand is a single-stranded nucleic acid, and wherein said step of detecting the complex comprises: contacting the complex with a lateral flow device or micro-array plate, wherein the lateral flow device or the micro-array plate comprises a capture/test region comprising a nucleic acid strand immobilized thereon, wherein the immobilized nucleic acid strand comprises a nucleotide sequence substantially complementary to a nucleotide sequence of the single-stranded nucleic acid capture ligand; and detecting a detectable signal from the reporter molecule.

In some embodiments of any of the aspects described herein, the first nucleic acid strand comprises the capture ligand, and the amplicon comprises the reporter molecule, and wherein the capture ligand is a toehold domain, and wherein said step of detecting the complex comprises: contacting the complex with a lateral flow device or micro-array plate, wherein the lateral flow device or the micro-array plate comprises a capture/test region comprising a nucleic acid strand immobilized thereon, wherein the immobilized nucleic acid strand comprises a nucleotide sequence substantially complementary to a region of the toehold domain; and detecting a detectable signal from the reporter molecule.

In some embodiments of any of the aspects described herein, the method comprises: contacting amplicons from amplification of a plurality of target nucleic acids with a plurality of probes to form a plurality of complexes, where each complex comprises a probe and an amplicon, wherein the amplicon comprises a reporter molecule, wherein each probe comprises a first nucleic acid strand and a molecule complexed with the first nucleic acid strand, the molecule capable of localizing a single-strand nucleic acid strand to a double-stranded nucleic acid or enhancing the kinetics of hybridization between two single-stranded nucleic acids, wherein the first nucleic acid strand comprises a binding domain comprising a nucleotide sequence substantially complementary to at least a first portion of an amplicon, wherein the first nucleic acid strand comprises a capture ligand, wherein the amplicon and the capture ligand in a first member of the plurality of complexes is different from the amplicon and the capture ligand in a second member of the plurality of complexes, and detecting the complex comprising the probe and the amplicon, wherein said step of detecting the complex comprises: contacting the plurality of complexes with a lateral flow device or micro-array plate, wherein the lateral flow device or the micro-array plate comprises a plurality of capture/test regions, each capture region comprising a capture probe immobilized thereon and capable of binding with capture ligand, wherein the capture probe in at least two capture/test regions bind a different capture ligand from each other; and detecting a detectable signal from the reporter molecules in the plurality of capture/test regions.

In another aspect, provided herein are compositions useful in detecting a target nucleic acid. In some embodiments the composition comprises a probe and an amplicon from amplification of the target nucleic acid, wherein the probe comprises a first nucleic acid strand and a molecule capable of localizing a single-stranded nucleic acid to a double-stranded nucleic acid or enhancing the kinetics of hybridization between two single-strand nucleic acids bound with the first nucleic acid strand, and wherein the first nucleic acid strand comprises a binding domain comprising a nucleotide sequence substantially complementary to at least a first portion of the amplicon.

In some embodiments, the composition comprises a probe and an amplicon from amplification of the target nucleic acid, wherein the probe comprises a first nucleic acid strand, a second nucleic acid strand, and a molecule capable of localizing a single-stranded nucleic acid to a double-stranded nucleic acid or enhancing the kinetics of hybridization between two single-strand nucleic acids, wherein the first nucleic acid strand comprises a binding domain comprising a nucleotide sequence substantially complementary to at least a first portion of amplicon, wherein the first nucleic acid strand comprises a first hybridization domain linked with the binding domain and the second nucleic acid strand is hybridized with the first hybridization domain of the first nucleic acid strand, and wherein the first nucleic acid strand comprises a reporter molecule capable of producing a detectable signal and the second nucleic acid strand comprises a capture ligand. Optionally, the first nucleic acid strand and the second nucleic acid strand hybridized with each other to form a double-stranded structure comprising a single-stranded loop region, wherein the first nucleic acid strand comprises a first hybridization domain linked to the binding domain linked to a second hybridization domain.

In some embodiments of any one of the aspects, the composition comprises a first probe, a second probe, and an amplicon from amplification of a target nucleic acid, wherein the first probe comprises a first nucleic acid strand and a first molecule localizing a single-stranded nucleic acid to a double-stranded nucleic acid or enhancing the kinetics of hybridization between two single-strand nucleic acids, and wherein the first nucleic acid strand of the first probe comprises a binding domain comprising a nucleotide sequence substantially complementary to at least a first portion of the amplicon. Optionally, the second probe comprises a first nucleic acid strand and a first molecule capable of localizing a single-stranded nucleic acid to a double-stranded nucleic acid or enhancing the kinetics of hybridization between two single-strand nucleic acids, and wherein the first nucleic acid strand of the second probe comprises a binding domain comprising a nucleotide sequence substantially complementary to at least a second portion of the amplicon, and wherein one of the first nucleic acid strand of the first probe and the first nucleic acid strand of the second probe comprises a reporter molecule capable of producing a detectable signal and the other of the first nucleic acid strand of the first probe and the first nucleic acid strand of the second probe comprises a capture ligand.

In some embodiments, the composition comprises a probe and an amplicon from amplification of a target nucleic acid, wherein the probe comprises a first nucleic acid strand, a second nucleic acid strand, and a first molecule capable of capable of localizing a single-stranded nucleic acid to a double-stranded nucleic acid or enhancing the kinetics of hybridization between two single-strand nucleic acids, and wherein the first nucleic acid strand of the first probe comprises a binding domain comprising a nucleotide sequence substantially complementary to at least a first portion of the amplicon, wherein the first nucleic acid strand comprises a first hybridization domain linked with the binding domain and the second nucleic acid strand is hybridized with the first hybridization domain of the first nucleic acid strand. Optionally, one of the first and second nucleic strand comprises a reporter molecule capable of producing a detectable signal and the other of the first and second nucleic strand comprises a quencher molecule, and wherein the quencher molecule quenches the detectable signal from the reporter molecule when the first and second nucleic acid strands are hybridized with each other, wherein the first hybridization domain and the binding domain of the first nucleic acid strand together comprise a nucleotide sequence substantially complementary to at least a portion of the amplicon.

In some embodiments, the composition further comprises one or more reagents for preparing an amplicon from a target nucleic acid.

Another aspect of the technology described herein relates to kits for detecting a target nucleic acid. Described herein are kit components that can be included in one or more of the kits described herein. The kit can comprise any of the compositions provided herein and packaging and materials therefore. Accordingly, in some embodiments the kit comprises a primer set for preparing an amplicon from a target nucleic acid, and a first probe, wherein the first probe comprises a first nucleic acid strand and a molecule capable of localizing a single-strand nucleic acid strand to a double-stranded nucleic acid or enhancing the kinetics of hybridization between two single-strand nucleic acids bound with the first nucleic acid strand, wherein the first nucleic acid strand comprises a binding domain comprising a nucleotide sequence substantially complementary to at least a first portion of the amplicon prepared from a target nucleic acid using the primer set. In some embodiments, the first nucleic acid strand comprises a reporter molecule capable of producing a detectable signal and at least one primer in the primer set comprises a capture ligand. In some other embodiments, the first nucleic acid strand comprises capture ligand and at least one primer in the primer set comprises a reporter molecule capable of producing a detectable signal. In some embodiments, the first nucleic acid strand comprises a first hybridization domain linked with the binding domain and the first probe further comprises a second nucleic acid strand hybridized with the first hybridization domain of the first nucleic acid strand.

In some embodiments, the kit further comprises a second probe, wherein the second probe comprises a first nucleic acid strand and a first molecule capable of localizing a single-strand nucleic acid strand to a double-stranded nucleic acid or enhancing the kinetics of hybridization between two single-strand nucleic acids, and wherein the first nucleic acid strand of the second probe comprises a binding domain comprising a nucleotide sequence substantially complementary to at least a second portion of the amplicon prepared from a target nucleic acid using the primer set. In some embodiments, one of the first nucleic acid strand of the first probe and the first nucleic acid strand of the second probe comprises a reporter molecule capable of producing a detectable signal and the other of the first nucleic acid strand of the first probe and the first nucleic acid strand of the second probe comprises a capture ligand.

In some embodiments of any of the aspects described herein, the molecule capable of localizing a single-stranded nucleic acid to a double-stranded nucleic acid or enhancing the kinetics of hybridization between two single-stranded nucleic acids is selected from the group consisting of recombinases, CRISPR-Cas proteins, single-stranded binding proteins, zinc finger nucleases, transcription activator-like effector nucleases (TALEN), site-specific recombinases, transcription factors, and any combinations thereof.

In some embodiments of any of the aspects described herein, the molecule capable of localizing a single-strand nucleic acid strand to a double-stranded nucleic acid or enhancing the kinetics of hybridization between two single-stranded nucleic acids is a recombinase or CRISPR-Cas protein. Generally, the molecule capable of localizing a single-strand nucleic acid strand to a double-stranded nucleic acid or enhancing the kinetics of hybridization between two single-stranded nucleic acids lacks nuclease activity.

In some embodiments of any of the aspects described herein, the molecule capable of localizing a single-strand nucleic acid strand to a double-stranded nucleic acid is a recombinase.

In some embodiments of any of the aspects described herein, the recombinase is selected from the group consisting of RecA, UvsX, RadA, Rad51, Dmcl, UvsY, Cre, Flp, Dre, SCre, VCre, Vika, B2, B3, KD, ΦC31, Bxb1, λ, HK022, HP1, γδ, ParA, Tn3, Gin, R4, TP901-1, TG1, PhiRv1, PhiBT1, SprA, XisF, TnpX, R, A118, spoIVCA, PhiMR11, SCCmec, TndX, XerC, XerD, XisA, Hin, Cin, mrpA, beta, PhiFC1, Fre, Clp, sTre, FimE, HbiFm, and homologues thereof, and modified versions thereof.

In some embodiments of any of the aspects described herein, the recombinase is RecA.

In some embodiments of any of the aspects described herein, the molecule capable of localizing a single-strand nucleic acid strand to a double-stranded nucleic acid or enhancing the kinetics of hybridization between two single-stranded nucleic acids is a CRISPR-Cas protein. Foe example, the molecule capable of localizing a single-strand nucleic acid strand to a double-stranded nucleic acid or enhancing the kinetics of hybridization between two single-stranded nucleic acids is a CRISPR-Cas protein selected from the group consisting of C2c1, C2c3, Cas1, Cas100, Cas12a, Cas12b, Cas12c, Cas12d, Cas12e, Cas13a, Cas13b, Cas13c, Cas1B, Cas2, Cas3, Cas4, Cas5, Cas6, Cas7, Cas8, Cas9 (also known as Csnl and Csxl2), Casl, CaslB, CaslO, Cmr1, Cmr3, Cmr4, Cmr5, Cmr6, Cpf1, Csa5, Csa5, CsaX, Csb1, Csb2, Csb3, Csc1, Csc2, Cse1, Cse2, Csf1, Csf2, Csf3, Csf4, Csm2, Csm3, Csm4, Csm5, Csm6, Csn2, Csx1, Csx10, Csx14, Csx15, Csx16, Csx17, Csx3, Csy1, Csy2, Csy3, and homologues thereof, or modified versions thereof. In some embodiments of any one of the aspects described herein, the CRISPR-Cas protein lack nuclease activity. In some embodiments of any of the aspects described herein, the CRISPR-Cas protein is Cas9. For example, the CRISPR-Cas protein is Cas9, and wherein the Cas9 lacks nuclease activity.

In some embodiments of any of the aspects described herein, the first nucleic acid strand comprises a first hybridization domain linked with the binding domain and the first probe comprises a second nucleic acid strand hybridized with the first hybridization domain of the first nucleic acid strand.

In some embodiments of any of the aspects described herein, the first hybridization domain is linked to the 5′-end of the binding domain.

In some embodiments of any of the aspects described herein, the first hybridization domain is linked to the 3′-end of the binding domain.

In some embodiments of any of the aspects described herein, the first nucleic acid strand comprises the reporter molecule and the amplicon comprises the capture ligand.

In some embodiments of any of the aspects described herein, the capture ligand is attached to the 5′-end of the nucleic acid strand it is attached to.

In some embodiments of any of the aspects described herein, the capture ligand is attached to the 3′-end of the nucleic acid strand it is attached to.

In some embodiments of any of the aspects described herein, the capture ligand is at an internal position of the nucleic acid strand it is attached to.

In some embodiments of any of the aspects described herein, the amplicon comprises at least one second capture ligand.

In some embodiments of any of the aspects described herein, one capture ligand is at the 5′-end and one capture ligand is the 3′-end of the nucleic acid strand they are attached to.

In some embodiments of any of the aspects described herein, one capture ligand is at the 5′-end and one capture ligand is at an internal position of the nucleic acid strand they are attached to.

In some embodiments of any of the aspects described herein, one capture ligand is at the 3′-end and one capture ligand is at an internal position of the nucleic acid strand they are attached to.

In some embodiments of any of the aspects described herein, one capture ligand is at the 5′-end, one capture ligand is at the 3′-end, and one capture ligand is at an internal position of the nucleic acid strand they are attached to.

In some embodiments of any of the aspects described herein, the amplicon comprises the reporter molecule and the first nucleic acid strand comprises the capture ligand.

In some embodiments of any of the aspects described herein, the amplicon further comprises a second reporter molecule.

In some embodiments of any of the aspects described herein, the capture ligand is attached to the 5′-end of the nucleic acid strand it is attached to.

In some embodiments of any of the aspects described herein, the capture ligand is attached to the 3′-end of the nucleic acid strand it is attached to.

In some embodiments of any of the aspects described herein detecting the reporter molecule comprises detecting a detectable signal produced by the reporter molecule.

In some embodiments of any of the aspects described herein, said step of detecting the complex comprises fluorescence detection, luminescence detection, chemiluminescence detection, colorimetric detection, immunofluorescence detection, or electrochemical detection.

In some embodiments of any of the aspects described herein, said step of detecting the complex comprises a lateral flow assay.

In some embodiments of any of the aspects described herein, said step of detecting the complex comprises: contacting the complex with a lateral flow device, wherein the lateral flow device or the micro-array plate comprises a capture/test region comprising a capture probe immobilized thereon, wherein the capture probe is capable of binding with capture label; and detecting the reporter molecule in the complex captured by the capture probe.

In some embodiments of any of the aspects described herein, said step of detecting the complex comprises micro-array detection.

In some embodiments of any of the aspects described herein, said step of detecting the complex comprises: contacting the complex with a micro-array plate, wherein the micro-array plate comprises a capture/test region comprising a capture probe immobilized thereon and capable of binding with capture label; and detecting the reporter molecule in the complex captured by the capture probe.

In some embodiments of any of the aspects described herein, the method comprises immobilizing the capture nucleic acid strand on the capture/test region prior to contacting with the complex.

In some embodiments of any of the aspects described herein, the capture nucleic acid comprises a capture ligand conjugated thereto and the capture/test region comprises a capture probe immobilized thereon, and wherein the capture probe is capable of binding with capture ligand of the capture nucleic acid strand.

In some embodiments of any of the aspects described herein, the capture nucleic acid comprises a molecule capable of localizing a single-strand nucleic acid strand to a double-stranded nucleic acid.

In some embodiments of any of the aspects described herein, the first and second portion of the amplicon are separated from each other by at least 5 nucleotides or at least 5 nucleotide base-pairs.

In some embodiments of any of the aspects described herein, the one of the first and second portion of the amplicon is on a first strand of the amplicon and the other of the first and second portion of the amplicon is on a second strand of the amplicon.

In some embodiments of any of the aspects described herein, the first and second portion of the amplicons are on the same strand of the amplicon.

In some embodiments of any of the aspects described herein, the capture ligand is linked at the 5′-end of the nucleic acid strand it is attached to.

In some embodiments of any of the aspects described herein, the capture ligand is linked at the 5′-end of the nucleic acid strand it is attached to.

In some embodiments of any of the aspects described herein, the reporter molecule and the quencher molecule are a FRET pair.

In some embodiments of any of the aspects described herein, the first nucleic acid strand comprises the reporter molecule.

In some embodiments of any of the aspects described herein, the second nucleic acid strand comprises the reporter molecule.

In some embodiments of any of the aspects described herein, the reporter molecule is attached to the 5′-end of the nucleic acid strand it is attached to.

In some embodiments of any of the aspects described herein, the reporter molecule is attached to the 3′-end of the nucleic acid strand it is attached to.

In some embodiments of any of the aspects described herein, the quencher molecule is attached to the 5′-end of the nucleic acid strand it is attached to.

In some embodiments of any of the aspects described herein, the quencher molecule is attached to the 3′-end of the nucleic acid strand it is attached to.

In some embodiments of any of the aspects described herein, said detecting the complex comprises fluorescence detection, luminescence detection, chemiluminescence detection, colorimetric detection, immunofluorescence detection, or electrochemical detection.

In some embodiments of any of the aspects described herein, the first nucleic acid strand comprises the capture ligand.

In some embodiments of any of the aspects described herein, the second nucleic acid strand comprises the capture ligand.

In some embodiments of any of the aspects described herein, the capture ligand is attached to the 5′-end of the nucleic acid strand it is attached to.

In some embodiments of any of the aspects described herein, the capture ligand is attached to the 3′-end of the nucleic acid strand it is attached to.

In some embodiments of any of the aspects described herein, the molecule capable of localizing a single-strand nucleic acid strand to a double-stranded nucleic acid is a CRISPR-Cas protein, the first nucleic acid strand is crRNA, and the second strand is a tracrRNA.

In some embodiments of any of the aspects described herein, the capture ligand is attached to the 5′-end of the nucleic acid strand it is attached to.

In some embodiments of any of the aspects described herein, the capture ligand is attached to the 3′-end of the nucleic acid strand it is attached to.

In some embodiments of any of the aspects described herein, the toehold domain is attached to the 5′-end of the nucleic acid strand it is attached to.

In some embodiments of any of the aspects described herein, the toehold domain is attached to the 3′-end of the nucleic acid strand it is attached to.

In some embodiments of any of the aspects described herein, the molecule capable of localizing a single-strand nucleic acid strand to a double-stranded nucleic acid or enhancing the kinetics of hybridization between two single-stranded nucleic acids is a CRISPR-Cas protein and the first nucleic acid strand is a guide RNA (sgRNA). In some further embodiments, the CRISPR-Cas protein lacks nuclease activity.

In some embodiments of any of the aspects described herein, the reporter molecule can be selected from the group consisting of fluorescent molecules, radioisotopes, chromophores, enzymes, enzyme substrates, chemiluminescent moieties, bioluminescent moieties, echogenic substances, non-metallic isotopes, optical reporters, paramagnetic metal ions, ferromagnetic metals, quantum dots (or semiconductor nanocrystals), nanoparticles (e.g. gold nanoparticles used in lateral flow assays, carbon nanoparticles), and latex and fluorescent beads.

In some embodiments of any of the aspects described herein, the reporter molecule is a fluorophore. Exemplary reporter molecules are described herein.

In some embodiments of any of the aspects described herein, the reporter molecule is 5-FAM.

In some embodiments of any of the aspects described herein, the reporter molecule is attached to the 5′-end of the nucleic acid strand it is attached to.

In some embodiments of any of the aspects described herein, the reporter molecule is attached to 3′-end of the nucleic acid strand it is attached to.

In some embodiments of any of the aspects described herein, the reporter molecule is a lateral flow detectable moiety.

In some embodiments of any of the aspects described herein, the nucleic acid strand comprising the reporter molecule comprises a second reporter molecule.

In some embodiments of any of the aspects described herein, one reporter molecule is at 5′-end and one reporter molecule is at 3′-end of nucleic acid strand they are attached to.

In some embodiments of any of the aspects described herein, the capture ligand is selected from the group consisting of binding pairs, nucleic acids, nucleosides and nucleotides, vitamins, hormones, proteins, peptides, peptidomimetics, amino acids, monosaccharides, disaccharides, oligosaccharides, polysaccharides, lipopolysaccharides, polyols, receptors, and ligands for a receptor.

In some embodiments of any of the aspects described herein, the capture agent is selected from the group consisting of binding pairs and nucleic acids.

In some embodiments of any of the aspects described herein, the capture ligand is biotin, or digoxigenin (dig).

In some embodiments of any of the aspects described herein, the capture ligand is a nucleic acid (e.g. a single stranded nucleic acid).

In some embodiments of any of the aspects described herein, the capture ligand is a nucleic acid and comprises a toehold domain.

In some embodiments of any of the aspects described herein, the capture ligand is attached to the 5′-end of the nucleic acid strand it is attached to.

In some embodiments of any of the aspects described herein, the capture ligand is attached to the 3′-end of the nucleic acid strand it is attached to.

In some embodiments of any of the aspects described herein, the method further comprising a step of amplifying the target nucleic acid to produce the amplicon.

In some embodiments of any of the aspects described herein, said step of amplifying the target nucleic acid to produce the amplicon comprises isothermal amplification. Exemplary, isothermal amplifications include, but is not limited to, Recombinase Polymerase Amplification (RPA), Loop Mediated Isothermal Amplification (LAMP), Helicase-dependent isothermal DNA amplification (HDA), Rolling Circle Amplification (RCA), Nucleic acid sequence-based amplification (NASBA), strand displacement amplification (SDA), nicking enzyme amplification reaction (NEAR), Polymerase Spiral Reaction (PSR), Hybridization Chain Reaction (HCR), Primer Exchange Reaction (PER), Signal Amplification by Exchange Reaction (SABER), transcription-based amplification system (TAS), Self-sustained sequence replication reaction (3 SR), Single primer isothermal amplification (SPIA), and cross-priming amplification (CPA).

In some embodiments of any of the aspects described herein, the steps of step of amplifying the target nucleic acid to produce the amplicon and contacting the amplicon with the first probe are simultaneous.

In some embodiments of any of the aspects described herein, the steps of step of amplifying the target nucleic acid to produce the amplicon and contacting the amplicon with the first probe are sequential.

In some embodiments of any of the aspects described herein, each hybridization domain is independently from about 5 nucleotides to about 100 nucleotides in length. For example, each hybridization domain is independently from about 10 nucleotides to about 75 nucleotides in length. In some embodiments of any of the aspects described herein, each hybridization domain is independently from about 15 nucleotides to about 50 nucleotides in length.

In some embodiments of any of the aspects described herein, each binding domain is independently from about 5 nucleotides to about 100 nucleotides in length. For example, each binding domain is independently from about 10 nucleotides to about 75 nucleotides in length. In some embodiments of any of the aspects described herein, each binding domain is independently from about 15 nucleotides to about 50 nucleotides in length.

In some embodiments of any of the aspects described herein, the target nucleic acid is single-stranded. In some embodiments of any of the aspects described herein, the target nucleic acid is double-stranded. In some embodiments of any of the aspects described herein, the target nucleic acid is DNA. In some embodiments of any of the aspects described herein, the target nucleic acid is RNA. In some embodiments of any of the aspects described herein, the method further comprises a step of preparing a cDNA from the target nucleic acid prior to preparing the amplicon.

In some embodiments of any of the aspects described herein, the amplicon is single-stranded. In some embodiments of any of the aspects described herein, the amplicon is double-stranded.

In some embodiments of any of the aspects described herein, the first nucleic acid strand comprises two or more hybridization domains. Optionally, the two or more hybridization domains are separated by a non-hybridizing domain.

In some embodiments of any of the aspects described herein, the first nucleic acid strand comprises two or more binding domains. Optionally, the two or more binding domains are separated by a non-hybridizing domain.

In some embodiments of any of the aspects described herein, the first nucleic acid strand and the second nucleic acid strand hybridized with each other to form a double-stranded structure comprising a single-stranded loop region, wherein the first nucleic acid strand comprises a first hybridization domain linked to the binding domain linked to a second hybridization domain, wherein the second nucleic acid strand comprises a first hybridization domain linked to a non-hybridizing domain linked to a second hybridization domain, wherein the first hybridization domain of the first nucleic acid strand and the second hybridization domain of the second nucleic acid strand are hybridized with each other forming a first double-stranded region, wherein the second hybridization domain of the first nucleic acid strand and the first hybridization domain of the second nucleic acid strand are hybridized with each other forming a second double-stranded region, wherein the binding domain of the first nucleic acid strand and the non-hybridizing domain of the second nucleic acid strand do not hybridize to each other.

In some embodiments of any of the aspects described herein, the first and second portion of the amplicons are on the same strand of the amplicon. In some other embodiments, the first and second portion of the amplicons are on different strands of the amplicon

In some embodiments of any of the aspects described herein, the first nucleic acid strand comprises a first hybridization domain linked with the binding domain linked with a second hybridization domain, wherein the second nucleic acid strand comprises a first hybridization domain linked with a non-hybridizing domain linked with a second hybridization domain, wherein the first hybridization domain of the first nucleic acid strand and the second hybridization domain of the second nucleic acid strand are hybridized with each other forming a first double-stranded region, wherein the second hybridization domain of the first nucleic acid strand and the first hybridization domain of the second nucleic acid strand are hybridized with each other forming a second double-stranded region, wherein the binding domain of the first nucleic acid strand and the non-hybridizing domain of the second nucleic acid strand do not hybridize to each other.

In some embodiments of any of the aspects described herein, the molecule capable of localizing a single-strand nucleic acid strand to a double-stranded nucleic acid enhancing the kinetics of hybridization between two single-stranded nucleic acids is a recombinase.

In some embodiments of any of the aspects described herein, the first nucleic acid strand comprises a first hybridization domain linked to the binding domain, and the first probe further comprises a second nucleic acid strand hybridized with the hybridization domain of the first strand, wherein the first or second nucleic strand comprises the capture ligand, wherein the amplicon comprises the reporter molecule capable of producing a detectable signal, and wherein the molecule capable of localizing a single-strand nucleic acid strand to a double-stranded nucleic acid or enhancing the kinetics of hybridization between two single-strand nucleic acids is a CRISPR-Cas protein, the first nucleic acid strand is crRNA, and the second strand is a tracrRNA, optionally, the CRISPR-Cas protein lacks nuclease activity.

In some embodiments of any of the aspects described herein, the first nucleic acid strand comprises the capture ligand, and the amplicon comprises the reporter molecule, wherein the capture ligand is a toehold domain, and wherein the molecule capable of localizing a single-strand nucleic acid strand to a double-stranded nucleic acid or enhancing the kinetics of hybridization between two single-strand nucleic acids is a CRISPR-Cas protein and the first nucleic acid strand is a guide RNA (sgRNA), optionally, the CRISPR-Cas protein lacks nuclease activity.

In some embodiments of any of the aspects described herein, the reporter molecule is a fluorophore. For example, the reporter molecule is a 5-FAM.

In some embodiments of any of the aspects described herein, the reporter molecule is attached to the 5′-end of the nucleic acid strand it is attached to. In some other embodiments of any of the aspects described herein, the reporter molecule is attached to the 3′-end of the nucleic acid strand it is attached to.

In another embodiment of any of the aspects described herein, the reporter molecule is a lateral flow detectable moiety.

In some embodiments of any of the aspects described herein, the nucleic acid strand comprising the reporter molecule further comprises a second reporter molecule.

In some embodiments of any of the aspects described herein, one reporter molecule is attached to the 5′-end and one reporter molecule is attached to the 3′-end of the nucleic acid strand they are attached to.

In some embodiments of any of the aspects described herein, the capture ligand is biotin or digoxigenin.

In some embodiments of any of the aspects described herein, the capture ligand is a nucleic acid and comprises a toehold domain.

In some embodiments of any of the aspects described herein, wherein the amplicon comprises at least one second capture ligand.

### BRIEF DESCRIPTION OF THE DRAWINGS

FIGS. 1A-1B show a schematic of single target detection assay through RecA-assisted DNA binding and isothermal amplification using either biotin (FIG. 1A) or FAM (FIG. 1B) as the detection label on the ssDNA.

FIG. 2A is a schematic of the SARS-CoV2 virus and the RdRp region that is being targeted.

FIG. 2B is a flowchart that shows how SARS-CoV2 is detected using either a two-step RecA or one-pot RecA method.

FIG. 3A-3B examines the screening of probes in one-pot RecA/RT-LAMP assays with a reference sequence (Ref2A_ACTB) using either short or long probes and biotin (FIG. 3A) or FAM (FIG. 3B) as the probe modification.

FIG. 4 examines the screening of probes with either a single modification or a dual modification.

FIG. 5A-5B examines the screening of probes in one-pot RecA/RT-LAMP assays with a reference sequence (Ref2A) using either short or long probes and biotin (FIG. 5A) or FAM (FIG. 5B) as the probe modification.

FIG. 6 depicts the detection of the spike sequence using a lateral flow assay using either short or long probes and FAM or biotin as the probe modification.

FIG. 7 depicts the detection of the RdRp sequence using a lateral flow assay using either short or long probes and FAM or biotin as the probe modification.

FIG. 8 depicts the detection of the nucleocapsid sequence using a lateral flow assay using long probes and FAM as the probe modification.

FIG. 9 examines the copy number limit of detection of the nucleocapsid sequence using short probes in a lateral flow assay.

FIG. 10 examines the copy number limit of detection of the nucleocapsid sequence using short probes in a lateral flow assay.

FIGS. 11A-11C examines the limit of detection of the actin beta (ACTB) mRNA sequence on a lateral flow strip after a 30-minute reaction (FIG. 11A), a 20-minute reaction (FIG. 11B), and a 10-minute reaction (FIG. 11C), with titrated concentrations of input ACTB mRNA.

FIG. 11D examines the copy number limit of detection of the actin beta mRNA sequence using long probes in a lateral flow assay.

FIGS. 12A-12C: FIG. 12A demonstrates targeting two double-stranded DNA target regions of LAMP amplicon with two distinct probes, each modified with a different moiety. FIG. 12B-12C examines the detection of SARS-CoV2 mRNA using either biotin as the forward probe (FIG. 12B) or FAM as the forward probe (FIG. 12C)

FIGS. 13A-13C shows the detection of exon-exon junctions on a lateral flow assay. FIG. 13A shows XPC, LRRC37A, and CHRNE. FIG. 13B shows MRS2, FAM98B, and C1QBP. FIG. 13C show FAM209A, MTAP, MXD2, TMEM140, PPP1R15A, CYBC1, and TSN.

FIGS. 14A-14F shows the detection of exon-exon junctions on a gel electrophoresis after a 15-minute or 30-minute reaction for MTAP_05-06 (FIG. 14A), MXD3_01-02 (FIG. 14B), TMEM140_01-02 (FIG. 14C), PPP1R15A_02-03 (FIG. 14D), CYBC1_04-05 (FIG. 14E), and TSN01-02 (FIG. 14F).

FIG. 15 shows the detection of different genes on both a lateral flow assay and gel electrophoresis at titrated concentrations of input exon-exon junction mRNA.

FIG. 16 shows the limit of detection of different genes on both a lateral flow assay and gel electrophoresis at titrated concentrations of input exon-exon junction mRNA, after a 15-minute reaction incubation.

FIGS. 17A-17C shows the limit of detection of different genes on both a lateral flow assay and gel electrophoresis at titrated concentration of input exon-exon junction mRNA, after a 30-minute reaction incubation.

FIG. 18 depicts the detection of amplicon in the presence of a probe featuring a poly T spacer next to the FAM modification at either the 3′ or 5′ end.

FIG. 19A shows a schematic of a mismatch probe proximal from the FAM detection agent.

FIG. 19B depicts the detection of the mismatch probe proximal from the FAM detection agent when the length of the mismatched section is increased.

FIG. 19C shows a schematic of a mismatch probe distal from the FAM detection agent.

FIG. 19D depicts the detection of the mismatch probe distal from the FAM detection agent when the length of the mismatched section is increased.

FIG. 20A shows a schematic of a SNP mutation probe.

FIG. 20B depicts the detection of the SNP mutation probe on a lateral flow assay when the number of SNPs is increased.

FIG. 20C shows a schematic of a probe with a mismatch region central to the probe binding region.

FIG. 20D depicts the detection of a probe with a mismatch region central to the probe binding region when length of the mismatched section is increased.

FIG. 21A examines a schematic of probes with two distinct binding domains, modified with both biotin and FAM.

FIG. 21B shows how a capture and reporter probe can bind to dsDNA.

FIG. 22A-22B show the dual reporter/capture probe detection scheme on a lateral flow assay using different templates. FIG. 22A shows templates directed to one gene, while FIG. 22B shows templates directed to another gene.

FIG. 23 depicts the steps needed to prepare to use both the reporter and capture probes in a lateral flow assay when the capture probe is pre-deposited on the lateral flow strip.

FIG. 24A-24B show the detection on a lateral flow assay using different templates. FIG. 24A shows templates directed to one gene, while FIG. 24B shows templates directed to another gene.

FIG. 25A-25B show the limit of detection on a lateral flow assay using different concentrations of template 2, using a dual reporter/capture probe detection scheme, with capture probe pre-deposited on the lateral flow strip. FIG. 25A shows a range of template amounts from 40 nM to 10.24 fM. FIG. 25B shows a range of template amounts from 200 nM to 10.24 fM.

FIG. 26A-26B shows the specificity of the probes using an unrelated target (FIG. 26A) or a partially related target (FIG. 26B).

FIG. 27 is a schematic that shows how the RecA/probe filament complex associates with the PCR amplicon and forms a complex by releasing the fluorophore strand.

FIG. 28A shows a schematic of multiplexed testing through RecA-based DNA binding-mediated strand displacement.

FIG. 28B shows the detection on lateral flow assays using RecA-based DNA binding-mediated strand displacement.

FIG. 29A shows a schematic of dual mismatch binding of multiplexed testing through RecA-based DNA binding-mediated strand displacement.

FIG. 29B examines detection on lateral flow assay when the mismatching is increased and when the incubation time is increased to 25 minutes after a 13-nt mismatch.

FIG. 30A shows a schematic of a mismatch probe proximal to the FAM detection agent and the detection of the mismatch probe proximal to the FAM detection agent when mismatches are increased.

FIG. 30B shows a schematic of a mismatch probe distal from the FAM detection agent and depicts the detection of the mismatch probe distal from the FAM detection agent when mismatches are increased.

FIG. 31 is a schematic on how to detect exon-exon junctions using the probes in a lateral flow assay.

FIG. 32 depicts a two-pot recombinase-based detection strategy workflow and the amount of time of detection for reverse transcriptase recombinase polymerase amplification (RT-PA), RecA incubation, and lateral flow immunoassay.

FIG. 33 examines how lateral flow immunoassays allow for amplified detection of an amplified product.

FIG. 34 depicts how ssDNA probes bind to synthetic DNA templates and are visible using a lateral flow assay.

FIG. 35 analyzes the screening of six primer pairs for each biomarker to identify those optimal for recombinase polymerase amplification.

FIG. 36 shows the lowest limit of detection using a lateral flow assay for two-pot RT-PA/RecA detection strategy.

FIG. 37 shows the lowest limit of detection using a lateral flow assay for one-pot RT-PA/RecA detection strategy.

FIG. 38 depicts a schematic showing Cas9 guides can be modified at the location of the tetraloop, for example the guide can be extended and labeled with a biotin to enable capture on a lateral flow assay.

FIG. 39 shows the recipe and readout with a lateral flow assay after targeting XPC exon-exon junction 4-5 in technical replicates.

FIG. 40 shows the recipe and readout with a lateral flow assay after targeting XPC exon-exon junction 4-5 in technical replicates with and without dCas9.

FIG. 41 examines the time to readout in a lateral flow assay after targeting XPC exon-exon junction 4-5 in technical replicates.

FIG. 42 shows the readout with a lateral flow assay after targeting LRRC37A exon-exon junction 7-8 in technical replicates with three different guide RNAs (gRNAs).

FIG. 43 examines time to readout for either XPC or LRRC37A using a lateral flow assay.

FIG. 44 depicts the screening of the detection of PPP6R3 using three different gRNAs on a lateral flow assay.

FIG. 45 depicts a schematic of how dCas9 single guides (sgRNA) can be modified at the 3′ end, for example the guide can be extended with a barcode region to enable capture by hybridization. In this case the capture strand hybridizes to a biotinylated capture probe for detection on a lateral flow assay.

FIG. 46 shows the recipe and readout with a lateral flow assay of a dCas9-RPA product captured with a pre-annealed capture probe.

FIG. 47 shows the recipe and readout with a lateral flow assay of a dCas9-RPA product captured with a capture probe in solution.

FIG. 48 examines the orthogonality test of the barcoded sgRNA against capture probes.

FIG. 49 depicts a schematic of how 3′ modified dCas9 sgRNA can be used for the simultaneous capture of orthogonal targets. For example, four different targets can be detected using four sgRNAs containing four orthogonal capture barcodes. By flowing the sample on a lateral flow pre-labeled with the respective probe sequences, each target can be detected via hybridization.

FIG. 50 depicts a schematic of how the use of toehold structures in the modified guide regions may enhance specificity by reducing non-specific binding due to the blocking of the capture nucleotides via the hairpin structure. Once the capture probe begins to hybridize it can invade the hairpin to stabilize the interaction.

FIG. 51 depicts a schematic of how this barcoded RNA strategy can also be expanded to a microarray format for improved readout of several targets. DNA amplicons with fluorescent labels can be labeled with barcoded dCas9 RNPs in solution. This can then be flowed past a microarray pre-labeled with capture probes. The binding of the DNA amplicons via the dCas9 RNPs enables readout of the relative abundance of each target nucleic acid.

FIG. 52A-52B depicts a schematic of how Cas9 guide RNA can be modified to incorporate barcodes. (FIG. 52A) depicts a standard Cas9 sgRNA (FIG. 52B) depicts three locations where extended sequences can be added without impacting Cas9 functionality: the 3′ end, 5′ end, and replacing the tetraloop.

### DETAILED DESCRIPTION

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference in their entirety for any purpose.

In embodiments described herein is a method for detecting a target nucleic acid. Generally, the method comprises: (i) contacting a target nucleic acid with a probe to form a complex comprising the probe and the target nucleic acid, wherein the probe comprises a nucleic acid strand and a molecule capable of stabilizing or enhancing interactions between two nucleic acids; and (ii) detecting the complex comprising the probe and the target nucleic acid. The nucleic acid strand of the probe comprises a binding domain comprising a nucleotide sequence substantially complementary to at least a first portion of the target molecule. Generally, the molecule capable of stabilizing or enhancing interactions between two nucleic acids is a molecule capable of localizing, binding, hybridizing or enhancing the kinetics of hybridization between two single-strand nucleic acids or between a single-stranded nucleic acid and a double-stranded nucleic acid. In some embodiments of any one of the aspects described herein, the molecule capable of stabilizing or enhancing interactions between two nucleic acids is capable of localizing, binding, hybridizing or enhancing the kinetics of hybridization between two-single-stranded nucleic acids. In some embodiments of any one of the aspects described herein, the molecule capable of stabilizing or enhancing interactions between two nucleic acids is capable of localizing, binding, hybridizing or enhancing the kinetics of hybridization between two-single-stranded nucleic acid and a double-stranded nucleic acid. For example, the molecule is capable of localizing, binding, hybridizing or enhancing the kinetics of hybridization between a single-stranded nucleic acid and one of the strands of a double-stranded nucleic acid.

In embodiments of the various aspects described herein, the complex comprising the probe and target nucleic acid is detected using a reporter molecule conjugated to a component in the complex comprising the probe and the target nucleic acid. For example, one of the nucleic acid strands of the probe or the target nucleic acid, e.g., an amplicon from amplification of a target nucleic acid can comprise a reporter molecule. In some embodiments, the nucleic acid strand of the probe comprises a reporter molecule. In some other embodiments, the target nucleic acid, e.g., an amplicon from amplification of the target nucleic acid comprises a reporter molecule.

### Stabilizing Molecules

Embodiments of the various aspects described herein include a molecule capable of stabilizing or enhancing interactions between two nucleic acids. In some embodiments of any one of the aspects described herein, the molecule capable of stabilizing or enhancing interactions between two nucleic acids can be a recombinase. Exemplary recombinases include, but are not limited to, RecA, UvsX, RadA, Rad51, Dmcl, UvsY, and homologues thereof, and modified versions thereof. In some embodiments, the recombinase can be a site-specific recombinase. Exemplary recombinases that are site specific include, but are not limited to, Cre, Flp, Dre, SCre, VCre, Vika, B2, B3, KD, ΦC31, Bxb1, λ, HK022, HP1, γδ, ParA, Tn3, Gin, R4, TP901-1, TG1, PhiRv1, PhiBT1, SprA, XisF, TnpX, R, A118, spoIVCA, PhiMR11, SCCmec, TndX, XerC, XerD, XisA, Hin, Cin, mrpA, beta, PhiFC1, Fre, Clp, sTre, FimE, HbiFm, and homologues thereof, and modified versions thereof. It is noted that the recombinase can be from an analog or variant of a known recombinase protein. In some preferred embodiments, the recombinase is RecA or a homologue or modified version thereof.

In some embodiments of any one of the aspects described herein, the molecule capable of stabilizing or enhancing interactions between two nucleic acids is a sequence guides nuclease, e.g., a sequence guided nuclease. When the is the molecule capable of stabilizing or enhancing interactions between two nucleic acids is a sequence guides nuclease, it preferably lacks any nuclease activity. One exemplary class of sequence guidance endonuclease is a CRISPR-Cas protein. Accordingly, in some embodiments of any one of the aspects described herein, the molecule capable of stabilizing or enhancing interactions between two nucleic acids is a CRISPR-Cas protein. In embodiments of the various aspects described herein, the CRISPR-Cas protein lacks any endonuclease activity. CRISPR-Cas proteins that lack any endonuclease activity are also referred as dCas herein. For example, the sequence guided endonuclease is catalytically inactive. In other words, the sequence guided endonuclease lacks nuclease, e.g., endonuclease activity of the parent CRISPR-Cas protein. Exemplary CRISPR-Cas protein selected from the group consisting of C2c1, C2c3, Cas1, Cas100, Cas12a, Cas12b, Cas12c, Cas12d, Cas12e, Cas13a, Cas13b, Cas13c, Cas1B, Cas2, Cas3, Cas4, Cas5, Cas6, Cas7, Cas8, Cas9 (also known as Csnl and Csxl2), Casl, CaslB, CaslO, Cmr1, Cmr3, Cmr4, Cmr5, Cmr6, Cpf1, Csa5, Csa5, CsaX, Csb1, Csb2, Csb3, Csc1, Csc2, Cse1, Cse2, Csf1, Csf2, Csf3, Csf4, Csm2, Csm3, Csm4, Csm5, Csm6, Csn2, Csx1, Csx10, Csx14, Csx15, Csx16, Csx17, Csx3, Csy1, Csy2, Csy3, and homologues thereof, or modified versions thereof. It is noted that the sequence guided endonuclease can be from an analog or variant of a known CRISPR-Cas protein. In some embodiments of any one of the aspects described herein, the molecule capable of stabilizing or enhancing interactions between two nucleic acids is Cas9 or a homologue or modified version thereof. For example, the molecule capable of stabilizing or enhancing interactions between two nucleic acids is Cas or a homologue or modified version thereof and wherein the Cas9 lacks endonuclease activity, i.e., the molecule capable of stabilizing or enhancing interactions between two nucleic acids is dCas9.

In some embodiments of any one of the aspects described herein, the molecule capable of stabilizing or enhancing interactions between two nucleic acids is a single-stranded binding (SSB) protein. Exemplary SSB proteins include, but are not limited to, E. coli SSB. T4 gp32, T7 gene 2.5 SSB, phage phi 29 SSB, RB69 bacteriophage gp32 protein, or a homologue or modified version thereof.

In some embodiments of any one of the aspects described herein, the molecule capable of stabilizing or enhancing interactions between two nucleic acids can be a zinc finger protein. Exemplary zince finger nucleases include, but are not limited to, members of the KU, Sp1, nuclear hormone receptor, and GATA protein families.

In some embodiments of any one of the aspects described herein, the molecule capable of stabilizing or enhancing interactions between two nucleic acids can be a transcription activator-like effector nucleases (TALEN).

It is noted that the molecule capable of stabilizing or enhancing interactions between two nucleic acids can be from any species.

### Reporter Molecules

Embodiments of the various aspects described herein include a reporter molecule. As used herein, the term “reporter” or “reporter molecule” refers to a molecule or composition capable of producing a detectable signal. Reporter molecules include any molecule, composition or moiety detectable by spectroscopic, photochemical, biochemical, immunochemical, electrical, optical or chemical means. Exemplary reporter molecules include, but are not limited, fluorescent molecules, radioisotopes, chromophores, enzymes, enzyme substrates, chemiluminescent moieties, bioluminescent moieties, echogenic substances, non-metallic isotopes, optical reporters, paramagnetic metal ions, ferromagnetic metals, quantum dots (or semiconductor nanocrystals), nanoparticles (e.g. gold nanoparticles used in lateral flow assays, carbon nanoparticles), and latex and fluorescent beads.

In some embodiments of any one of the aspects described herein, a reporter molecule can be a fluorescent dye molecule, or fluorophore. A wide variety of fluorescent reporter dyes are known in the art. Typically, the fluorophore is an aromatic or heteroaromatic compound and can be a pyrene, anthracene, naphthalene, acridine, stilbene, indole, benzindole, oxazole, thiazole, benzothiazole, cyanine, carbocyanine, salicylate, anthranilate, coumarin, fluorescein, rhodamine or other like compound.

In some embodiments, the reporter molecule is selected from the group consisting of 5-Carboxyfluorescein (5-FAM); 1,5 IAEDANS; 1,8-ANS; 4-Methylumbelliferone; 5-carboxy-2,7-dichlorofluorescein; 5-Carboxynapthofluorescein (pH 10); 5-Carboxytetramethylrhodamine (5-TAMRA); 5-FAM (5-Carboxyfluorescein); 5-Hydroxy Tryptamine (HAT); 5-ROX (carboxy-X-rhodamine); 5-TAMRA (5-Carboxytetramethylrhodamine); 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Aequorin (Photoprotein); Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; AMCA (Aminomethylcoumarin); AMCA-X; Aminoactinomycin D; Aminocoumarin; Anilin Blue; Anthrocyl stearate; APC-Cy7; APTS; Astrazon Brilliant Red 4G; Astrazon Orange R; Astrazon Red 6B; Astrazon Yellow 7 GFF; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Factamase; BFP blue shifted GFP (Y66H); BG-647; Bimane; Bisbenzamide; Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy FI; Bodipy FF ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Calcein; Calcein Blue; Calcium Crimson™; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CFDA; CFP Cyan Fluorescent Protein; Chlorophyll; Chromomycin A; Chromomycin A; CMFDA; Coelenterazine; Coelenterazine cp; Coelenterazine f; Coelenterazine fcp; Coelenterazine h; Coelenterazine hep; Coelenterazine ip; Coelenterazine O; Coumarin Phalloidin; CPM Methylcoumarin; CTC; Cy2™; Cy3.1 8; Cy3.5™; Cy3™; Cy5.1 8; Cy5.5™; Cy5™; Cy7™; Cyan GFP; cyclic AMP Fluorosensor (FiCRhR); d2; Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di-16-ASP); DIDS; Dihydorhodamine 123 (DHR); DiO (DiOC18(3)); DiR; DiR (DiIC18(7)); Dopamine; DsRed; DTAF; DY-630-NHS; DY-635-NHS; EBFP; ECFP; EGFP; EFF 97; Eosin; Erythrosin; Erythrosin ITC; Ethidium homodimer-1 (EthD-1); Euchrysin; Europium (III) chloride; Europium; EYFP; Fast Blue; FDA; Feulgen (Pararosaniline); FITC; FL-645; Flazo Orange; Fluo-3; Fluo-4; Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura-2, high calcium; Fura-2, low calcium; Genacryl Brilliant Red B; Genacryl Brilliant Yellow 10GF; Genacryl Pink 3G; Genacryl Yellow 5GF; GFP (S65T); GFP red shifted (rsGFP); GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indodicarbocyanine (DiD); Indotricarbocyanine (DiR); Intrawhite Cf; JC-1; JO-JO-1; JO-PRO-1; FaserPro; Faurodan; EDS 751; Feucophor PAF; Feucophor SF; FeucophorWS; Fissamine Rhodamine; Fissamine Rhodamine B; FOFO-1; FO-PRO-1; Fucifer Yellow; Mag Green; Magdala Red (Phloxin B); Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxadidole; Noradrenaline; Nuclear Fast Red; Nuclear Yellow; Nylosan Brilliant Iavin EBG; Oregon Green™; Oregon Green 488-X; Oregon Green™ 488; Oregon Green™ 500; Oregon Green™ 514; Pacific Blue; Pararosaniline (Feulgen); PE-Cy5; PE-Cy7; PerCP; PerCP-Cy5.5; PE-TexasRed (Red 613); Phloxin B (Magdala Red); Phorwite AR; Phorwite BKF; Phorwite Rev; Phorwite RPA; Phosphine 3R; PhotoResist; Phycoerythrin B [PE]; Phycoerythrin R [PE]; PKH26; PKH67; PMIA; Pontochrome Blue Black; POPO-1; POPO-3; PO-PRO-1; PO-PRO-3; Primuline; Procion Yellow; Propidium Iodid (PI); PyMPO; Pyrene; Pyronine; Pyronine B; Pyrozal Brilliant Flavin 7GF; QSY 7; Quinacrine Mustard; Resorufm; RH 414; Rhod-2; Rhodamine; Rhodamine 110; Rhodamine 123; Rhodamine 5 GFD; Rhodamine 6G; Rhodamine B 540; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycoerythrin (PE); red shifted GFP (rsGFP, S65T); S65A; S65C; S65F; S65T; Sapphire GFP; Serotonin; Sevron Brilliant Red 2B; Sevron Brilliant Red 4G; Sevron Brilliant Red B; Sevron Orange; Sevron Yellow F; sgBFP™; sgBFP™ (super glow BFP); sgGFP™; sgGFP™ (super glow GFP); SITS; SITS (Primuline); SITS (Stilbene Isothiosulphonic Acid); SPQ (6-methoxy-N-(3-sulfopropyl)-quinolinium); Stilbene; Sulphorhodamine B can C; Sulphorhodamine G Extra; Tetracycline; Tetramethylrhodamine; Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC (TetramethylRodaminelsoThioCyanate); True Blue; TruRed; Ultralite; Uranine B; Uvitex SFC; wt GFP; WW 781; XF665; X-Rhodamine; XRITC; Xylene Orange; Y66F; Y66H; Y66W; Yellow GFP; YFP; YO-PRO-1; YO-PRO-3; YOYO-1; and YOYO-3. Many suitable forms of these fluorescent compounds are available and can be used.

In some embodiments of any one of the aspects described herein, the reporter molecule is 5-FAM.

Other exemplary detectable labels include luminescent and bioluminescent markers (e.g., biotin, luciferase (e.g., bacterial, firefly, click beetle and the like), luciferin, and aequorin), radiolabels (e.g., 3H, 125I, 35S, 14C, or 32P), enzymes (e.g., galactosidases, glucorinidases, phosphatases (e.g., alkaline phosphatase), peroxidases (e.g., horseradish peroxidase), and cholinesterases), and calorimetric labels such as colloidal gold or colored glass or plastic (e.g., polystyrene, polypropylene, and latex) beads. Patents teaching the use of such labels include U.S. Pat. Nos. 3,817,837, 3,850,752, 3,939,350, 3,996,345, 4,277,437, 4,275,149, and 4,366,241, each of which is incorporated herein by reference.

In some embodiments of any of the aspects, a detectable label can be a radiolabel including, but not limited to ³H, ¹²⁵I, ³⁵S, ¹⁴C, ³²P, and ³³P. Suitable non-metallic isotopes include, but are not limited to, ¹¹C, ¹⁴C, ¹³N, ¹⁸F, ¹²³I, ¹²⁴I, and ¹²⁵I. Suitable radioisotopes include, but are not limited to, ⁹⁹mTc, ⁹⁵Tc, ¹¹¹In, ⁶²Cu, ⁶⁴Cu, Ga, ⁶⁸Ga, and ¹⁵³Gd. Suitable paramagnetic metal ions include, but are not limited to, Gd(III), Dy(III), Fe(III), and Mn(II). Suitable X-ray absorbers include, but are not limited to, Re, Sm, Ho, Lu, Pm, Y, Bi, Pd, Gd, La, Au, Au, Yb, Dy, Cu, Rh, Ag, and Ir.

In some embodiments of any one of the aspects described herein, the reporter molecule is a radionuclide and wherein the radionuclide is bound to a chelating agent or chelating agent-linker attached to the nucleic acid strand the reporter molecule is attached to. Exemplary chelating agents include, but are not limited to, diethylenetriaminepentaacetic acid (DTPA) and ethylenediaminetetraacetic acid (EDTA). Suitable radionuclides for direct conjugation include, without limitation, ³H, ¹⁸F, ¹²⁴I, ¹²⁵I, ¹³¹I, ³⁵S, ¹⁴C, ³²P, and ³³P and mixtures thereof. Suitable radionuclides for use with a chelating agent include, without limitation, ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹¹In, ¹¹⁷mSn, ¹⁴⁹Pm, ¹⁵³Sm, ¹⁶⁶Ho, ¹⁷⁷Lu, ¹⁸⁶Re, ¹⁸⁸Re, ²¹¹At, ²¹²Bi, and mixtures thereof. Suitable chelating agents include, but are not limited to, DOTA, BAD, TETA, DTPA, EDTA, NTA, HDTA, their phosphonate analogs, and mixtures thereof. One of skill in the art will be familiar with methods for attaching radionuclides, chelating agents, and chelating agent-linkers to molecules such as nucleic acids.

In some embodiments of any of the aspects, a reporter molecule can be an enzyme, e.g., an enzymatic label including, but not limited to horseradish peroxidase and alkaline phosphatase. An enzymatic label can produce, for example, a chemiluminescent signal, a color signal, or a fluorescent signal. Enzymes contemplated for use to detectably label an antibody reagent include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase. In some embodiments of any of the aspects, a reporter molecule is a chemiluminescent label, including, but not limited to lucigenin, luminol, luciferin, isoluminol, theromatic acridinium ester, imidazole, acridinium salt and oxalate ester. In some embodiments of any of the aspects, a reporter molecule can be a spectral colorimetric label including, but not limited to colloidal gold or colored glass or plastic (e.g., polystyrene, polypropylene, and latex) beads.

Means of detecting such labels are well known to those of skill in the art. Thus, for example, fluorescent markers can be detected using a photo-detector to detect emitted light, and radiolabels can be detected using photographic film or scintillation counters. Enzymatic labels are typically detected by providing the enzyme with an enzyme substrate and detecting the reaction product produced by the action of the enzyme on the enzyme substrate, and calorimetric labels can be detected by visualizing the colored label

It noted that a reporter molecule can be attached at any position of the nucleic acid strand it is attached to. For example, the reporter molecule can be attached at 5′-end of the nucleic acid strand it is attached to. Alternatively, the reporter molecule can be attached at 5′-end of the nucleic acid strand it is attached to. In yet another non-limiting alternative, the reporter molecule can be attached at an internal position of the nucleic acid strand it is attached to. In addition, the reporter molecule can be linked directly, i.e., via a bond or indirectly, e.g., via linker to the nucleic acid strand it is attached to.

In some embodiments of any one of the aspects described herein, the nucleic acid strand comprising the reporter molecule comprises two or more reporter molecules. When two or more reporter molecules are present in a nucleic acid strand, they can be the same or different. Further, they can be attached independently at any position of the nucleic acid strand. For example, in some embodiments, a nucleic acid strand comprises two reporter molecules, where one of the reporter molecules is attached at the 5′-end of the strand and the second reporter molecule is attached to the 3′-end. In some embodiments, a nucleic acid strand comprises two reporter molecules, where one reporter molecule is linked to the nucleic acid strand and the second reporter molecule is linked to the reporter molecule linked to the strand.

In some embodiments of any one of the aspects described herein, the reporter molecule is linked to a nucleic acid strand of the probe. In some other embodiments of any one of the aspects described herein, the reporter molecule is linked to the target nucleic acid, e.g., to an amplicon produced from the target nucleic acid.

### Capture Ligand

Embodiments of the various aspects described herein include a capture ligand. As used herein, a “capture ligand” refers to a molecule that can bind with any molecule or moiety that can bind with another molecule or moiety (a binding partner). In some embodiments of any one of the aspects, the capture ligand binds specifically with its binding partner. As used herein, the terms “binds specifically”, and “binding specificity” in reference to a ligand binding molecule refers to its capacity to bind to a given molecule or moiety preferentially over other molecules or moieties. For example, if the capture ligand (“molecule A”) is capable of “binding specifically” to a given ligand (“molecule B”), molecule A has the capacity to discriminate between molecule B and any other number of potential alternative binding partners. Accordingly, when exposed to a plurality of different but equally accessible molecules as potential binding partners, molecule A will selectively bind to molecule B and other alternative potential binding partners will remain substantially unbound by molecule A. In general, molecule A will preferentially bind to molecule B at least 10-fold, preferably 50-fold, more preferably 100-fold, and most preferably greater than 100-fold more frequently than other potential binding partners. Molecule A may be capable of binding to molecules that are not molecule B at a weak, yet detectable level. This is commonly known as background binding and is readily discernible from molecule B-specific binding, for example, by use of an appropriate control.

Exemplary capture ligands include, but are not limited to, one member of a binding pair, nucleic acids, nucleosides and nucleotides, vitamins, hormones, proteins, peptides, peptidomimetics, amino acids, monosaccharides, disaccharides, oligosaccharides, polysaccharides, lipopolysaccharides, polyols, receptors, ligand for a receptor, and the like. In some embodiments of any one of the aspects described herein, the capture ligand is one member of a binding pair. As used herein, the term “binding pair” refers to a pair of moieties that specifically bind each other with high affinity, generally in the low micromolar to picomolar range. Non-limiting examples of binding pairs include antigen:antibody (including antigen-binding fragments or derivatives thereof), biotin:avidin, biotin:streptavidin, biotin:neutravidin (or other variants of avidin that bind biotin), two complementary nucleic acid strands, receptor:ligand, and the like. Additional molecule for binding pair can include, neutravidin, strep-tag, strep-tactin and derivatives, and other peptide, hapten, dye-based tags-anti-Tag combinations such as SpyCatcher-SpyTag, His-Tag, Fc Tag, Digitonin, GFP, FAM, haptens, SNAP-TAG. HRP, FLAG, HA, myc, glutathione S-transferase (GST), maltose binding protein (MBP), small molecules, and the like.

In some embodiments of any one of the aspects described herein, the capture ligand is biotin or a derivative or analogue of biotin. In some embodiments of any one of the aspects described herein, the capture ligand is a digoxigenin.

In some embodiments of any one of the aspects described herein, the capture ligand is a nucleic acid (e.g. a single stranded nucleic acid). For example, the capture ligand is a nucleic acid (e.g. a single stranded nucleic acid) and wherein the capture ligand comprises a toehold domain.

It noted that a capture ligand can be attached at any position of the nucleic acid strand it is attached to. For example, the capture ligand can be attached at 5′-end of the nucleic acid strand it is attached to. Alternatively, the capture ligand can be attached at 5′-end of the nucleic acid strand it is attached to. In yet another non-limiting alternative, the capture ligand can be attached at an internal position of the nucleic acid strand it is attached to. In addition, the capture ligand can be linked directly, i.e., via a bond or indirectly, e.g., via linker to the nucleic acid strand it is attached to.

In some embodiments of any one of the aspects described herein, the nucleic acid strand comprising the capture ligand comprises two or more capture ligands. When two or more capture ligands are present in a nucleic acid strand, they can be the same or different. Further, they can be attached independently at any position of the nucleic acid strand. For example, in some embodiments, a nucleic acid strand comprises two capture ligands, where one of the capture ligands is attached at the 5′-end of the strand and the second capture is attached to the 3′-end. In some embodiments, a nucleic acid strand comprises two capture ligands, where one capture ligand is linked to the nucleic acid strand and the second capture ligand is linked to the capture ligand linked to the strand.

In some embodiments of any one of the aspects described herein, the capture ligand is linked to a nucleic acid strand of the probe. In some other embodiments of any one of the aspects described herein, the capture ligand is linked to the target nucleic acid, e.g., to an amplicon produced from the target nucleic acid.

In some embodiments of any one of the aspects described herein, a reporter molecule is linked to a nucleic acid strand of the probe and a capture ligand is linked to the target nucleic acid, e.g., to an amplicon produced from the target nucleic acid. In some other embodiments, a capture ligand is linked to a nucleic acid strand of the probe and a reporter molecule is linked to the target nucleic acid, e.g., to an amplicon produced from the target nucleic acid.

### Amplification

In some embodiments of any of the aspects, the method comprises a step of amplifying the target nucleic acid prior to contacting with the probe. In such embodiments, the amplicon can be the target nucleic acid being contacted with the probe. Thus, in some embodiments, the target nucleic acid contacted with the probe is a double-stranded or single-stranded amplicon from the amplification step. As used herein, the term “amplifying” refers to a step of submitting a nucleic acid sequence to conditions sufficient to allow for amplification of a polynucleotide if all of the components of the reaction are intact. Components of an amplification reaction include, e.g., primers, a polynucleotide template, polymerase, nucleotides, and the like. The term “amplifying” typically refers to an “exponential” increase in target nucleic acid. However, “amplifying” as used herein can also refer to linear increases in the numbers of a select target sequence of nucleic acid, such as is obtained with cycle sequencing. Methods of amplifying and synthesizing nucleic acid sequences are known in the art. For example, see U.S. Pat. Nos. 7,906,282, 8,367,328, 5,518,900, 7,378,262, 5,476,774, and 6,638,722, contents of all of which are incorporated by reference herein in their entirety.

In some embodiments, the amplification is selected from the group consisting of Recombinase Polymerase Amplification (RPA), Loop Mediated Isothermal Amplification (LAMP), Helicase-dependent isothermal DNA amplification (HDA), Rolling Circle Amplification (RCA), Nucleic acid sequence-based amplification (NASBA), strand displacement amplification (SDA), nicking enzyme amplification reaction (NEAR), polymerase Polymerase Spiral Reaction (PSR), Hybridization Chain Reaction (HCR), Primer Exchange Reaction (PER), Signal Amplification by Exchange Reaction (SABER), transcription-based amplification system (TAS), Self-sustained sequence replication reaction (3 SR), Single primer isothermal amplification (SPIA), and cross-priming amplification (CPA).

In some embodiments, the amplification is Loop-mediated Isothermal Amplification (LAMP). LAMP allows for the amplification of target DNA using strand displacement DNA synthesis using primer sets without the need for a thermocycler. In contrast to PCR techniques, LAMP provides high specificity, efficiency, and rapidity under isothermal conditions to amplify a target sequence. LAMP is described in detail, e.g. in Notomi T, et al. “Loop-mediated isothermal amplification of DNA.” Nucleic Acids Res. 2000; 28(12):E63, which is incorporated herein by reference in its entirety.

The advantage of the methods provided herein is that the step of contacting the probe with the target nucleic acid can be carried out simultaneously with amplification of the target nucleic acid. In other words, the amplification and contacting with the probe steps can be performed in a single reaction vessel.

In some embodiments, said step of contacting the target nucleic acid, i.e., the amplicon with the probe is after the amplification of the target nucleic acid. As a non-limiting example, said contacting with the probe can be performed at least 5 seconds, at least 10 seconds, at least 30 seconds, at least 45 seconds, at least 1 minute (min), at least 2 min, at least 3 min, at least 4 min, at least 5 min, at least 6 min, at least 7 min, at least 8 min, at least 9 min, at least 10 min, at least 20 min, at least 30 min, at least 40 min, at least 50 min, at least 60 min or more after the amplification.

In some embodiments, said contacting of the target nucleic acid, e.g., the amplicon with the probe is performed after isolation or purification of the amplicons from the amplification of the target nucleic acid. In other words, the method further comprises a step of isolating or purifying the amplicon from the amplification reaction prior to contacting with the probe.

The methods described herein allow fast detection of target nucleic acids. For example, the total time from starting the assay and detecting a signal can be few minutes to less than 2 hours. For clarity, starting the assay means adding the probe to the sample comprising the target nucleic acids. The total time from starting the assay and detecting a signal can be from about 5 minutes to about 90 minutes. Thus, the total time, from starting the assay to detecting a signal can be about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 70 minutes, about 75 minutes, about 80 minutes, or about 90 minutes. In some embodiments, the assay time from contacting the probe with the target nucleic acid and detecting a signal is from about 5 to about 15 minutes. For example, the assay time from contacting the probe with the target nucleic acid and detecting a signal is about 5 minutes, about 6 minutes, about 7 minutes, about 8 minutes, about 9 minutes, about 10 minutes, about 11 minutes, about 12 minutes, about 13 minutes, about 14 minutes, about 15 minutes.

When the method comprises an amplification step, e.g., a step of amplifying the target nucleic acid, the total time from starting the assay and detecting a signal can be few minutes to less than 2 hours. For clarity, starting the assay in this context means adding reagents to the sample for amplifying the target nucleic acids. The total time from starting the assay and detecting a signal can be from about 15 minutes to about 90 minutes. Thus, the total time, from starting the assay to detecting a signal can be about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 70 minutes, about 75 minutes, about 80 minutes, or about 90 minutes.

In some embodiments of any of the aspects, the total time for the methods described herein can be at most 15 minutes, at most 16 minutes, at most 17 minutes, at most 18 minutes, at most 19 minutes, at most 20 minutes, at most 21 minutes, at most 22 minutes, at most 23 minutes, at most 24 minutes, at most 25 minutes, at most 26 minutes, at most 27 minutes, at most 28 minutes, at most 29 minutes, at most 30 minutes, at most 31 minutes, at most 32 minutes, at most 33 minutes, at most 34 minutes, at most 35 minutes, at most 36 minutes, at most 37 minutes, at most 38 minutes, at most 39 minutes, at most 40 minutes, at most 41 minutes, at most 42 minutes, at most 43 minutes, at most 44 minutes, at most 45 minutes, at most 46 minutes, at most 47 minutes, at most 48 minutes, at most 49 minutes, at most 50 minutes, at most 51 minutes, at most 52 minutes, at most 53 minutes, at most 54 minutes, at most 55 minutes, at most 56 minutes, at most 57 minutes, at most 58 minutes, at most 59 minutes, at most 60 minutes, at most 70 minutes, at most 75 minutes, at most 80 minutes, or at most 90 minutes.

In some embodiments, the total time for the methods described herein can be from about 5 minutes to about 60 minutes. For example, the total time for the methods described herein can be from about 5 minutes to about 45 minutes. For example, the total time for the methods described herein can be from about 5 minutes to about 30 minutes, from about 5 minutes to about 25 minutes, from about 5 minutes to about 20 minutes, or from about 5 minutes to about 15 minutes. In some embodiment, the methods described herein can be from about 10 minutes to about 45 minutes, from about 10 minutes to about 30 minutes, from about 20 minutes to about 40 minutes, or from about 25 minutes to about 35 minutes.

The step of contacting the probe to the target nucleic acid can be performed at a temperature between from about 20° C. to about 75° C. For example, step of contacting the probe to the target nucleic acid can be performed at about 25° C. to about 70° C., from about 30° C. to about 65° C. or from about 35° C. to about 60° C. In some embodiments, the step of contacting the probe to the target nucleic acid can be performed at a temperature at 65° C. In some embodiments, the amplification, and the contacting steps are performed at a constant temperature.

### Detection

Embodiments of the various aspects described herein include a step of detecting the complex comprising the probe and the target nucleic acid. Generally, the detecting step comprises detecting a detectable signal produced by the reporter molecule linked to one of the components of the complex. Methods for detecting a detectable signal produced by a reporter molecule are well known and available to one of skill in the art. Exemplary methods include, but are not limited to, fluorescence detection, luminescence detection, chemiluminescence detection, colorimetric detection, immunofluorescence detection, or electrochemical detection.

In some embodiments of any one of the aspects described herein, the step of detecting the complex comprises capturing or immobilizing the complex on a surface and detecting a detectable signal produced by the reporter molecule. For example, a component in the complex comprises a capture ligand and the surface comprises a capture/test region comprising a capture probe immobilized thereon, wherein the capture probe is capable of binding with capture ligand. In some embodiments, the surface can be a surface of a diagnostic device. For example, the surface can be the surface of a lateral flow device or a micro-plate.

In some embodiments of any of the aspects, the reporter molecule can be detected using lateral flow detection, also known as a lateral flow immunoassay assay (LFIA), laminar flow, the immunochromatographic assay, or strip test. LFIAs are a simple device intended to detect the presence (or absence) of a target molecule, e.g. a reporter molecule. There are currently many LFIA tests used for medical diagnostics, either for home testing, point of care testing, or laboratory use. LFIA tests are a form of immunoassay in which the test sample flows along a solid substrate via capillary action. After the sample is applied to the test strip it encounters a colored reagent (generally comprising a capture probe capable of binding with the test target molecule) bound to microparticles which mixes with the sample and transits the substrate encountering lines or zones which have been pretreated with a capture probe that can bind with the capture ligand. Depending upon the level of target present in the sample the colored reagent can be captured and become bound at the test line or zone. LFIAs are essentially immunoassays adapted to operate along a single axis to suit the test strip format or a dipstick format. Strip tests are extremely versatile and can be easily modified by one skilled in the art for detecting an enormous range of antigens from fluid samples such as urine, blood, water, and/or homogenized tissue samples etc. Strip tests are also known as dipstick tests, the name bearing from the literal action of “dipping” the test strip into a fluid sample to be tested. LFIA strip tests are easy to use, require minimum training and can easily be included as components of point-of-care test (POCT) diagnostics to be used on site in the field. LFIA tests can be operated as either competitive or sandwich assays. Sandwich LFIAs are similar to sandwich ELISA. The sample first encounters colored particles which are labeled capture probes capable of binding with the test target molecule. The test line will also contain capture probes to the same target, although it may bind to a different part of the test target molecule. The test line will show as a colored band in positive samples. In some embodiments of any of the aspects, the lateral flow immunoassay can be a double sandwich assay, a competitive assay, a quantitative assay or variations thereof. Competitive LFIAs are similar to competitive ELISA. The sample first encounters colored particles which are labeled with the target antigen or an analogue. The test line contains antibodies to the target/its analogue. Unlabeled antigen in the sample will block the binding sites on the antibodies preventing uptake of the colored particles. The test line will show as a colored band in negative samples. There are a number of variations on lateral flow technology. It is also possible to apply multiple capture zones to create a multiplex test.

The use of lateral flow tests to detect nucleic acids have been described in the art; see e.g., U.S. Pat. Nos. 9,121,849; 9,207,236; and 9,651,549; the content of each of which is incorporated herein by reference in its entirety. The use of “dip sticks” or LFIA test strips and other solid supports have been described in the art in the context of an immunoassay for a number of targets. U.S. Pat. Nos. 4,943,522; 6,485,982; 6,187,598; 5,770,460; 5,622,871; 6,565,808, U.S. patent application Ser. No. 10/278,676; U.S. Ser. No. 09/579,673 and U.S. Ser. No. 10/717,082, which are incorporated herein by reference in their entirety, are non-limiting examples of such lateral flow test devices. Examples of patents that describe the use of “dip stick” technology to detect soluble antigens via immunochemical assays include, but are not limited to U.S. Pat. Nos. 4,444,880; 4,305,924; and 4,135,884; which are incorporated by reference herein in their entireties. The apparatuses and methods of these three patents broadly describe a first component fixed to a solid surface on a “dip stick” which is exposed to a solution containing a soluble antigen that binds to the component fixed upon the “dip stick,” prior to detection of the component-antigen complex upon the stick.

Typically, a lateral flow strip comprises: a sample pad, a conjugate pad, a detection membrane, and optionally an absorption pad. The sample pad is the first pad of the flow strip and it is the location where sample, e.g., the complex comprising the probe and the nucleic acid or an amplicon prepared from the target nucleic acid, is added. In some embodiments of any of the aspects, the sample pad comprises cellulose fiber filters and/or woven meshes. In some embodiments of any of the aspects, the sample pad further comprises a buffer. The conjugate pad is between the sample pad and the membrane; the conjugate pad comprises detector molecules, which are distributed into the membrane of the lateral flow strip after being contacted with the running buffer from the sample pad. In some embodiments of any of the aspects, the conjugate pad comprises glass fibers, cellulose fibers, and/or surface-modified polyester. In some embodiments of any of the aspects, the detection membrane is a nitrocellulose membrane, comprising the test line(s) and control lines(s). Absorbent pads, when used, are placed at the distal end of the lateral flow strip. The primary function of the absorbent pad is to increase the total volume of running buffer that enters the lateral flow strip.

The lateral flow assay can be carried out in a lateral flow device (LFD), i.e., a lateral flow test strip. The lateral flow device or strip comprises a test region. The test region comprises a capture probe capable of binding with the capture ligand in the complex immobilized therein. In some embodiments, the lateral flow device or strip also comprises a control region comprising a different capture probe immobilized therein. The capture probe in the control region can bind to a capture probe capable of binding the target molecule, e.g., the report molecule.

The conditions for the detection step depend on the specific assay. In some embodiments of any of the aspects, the lateral flow detection step is performed in at most 1 minute, at most 2 minutes, at most 3 minutes, at most 4 minutes, at most 5 minutes, at most 6 minutes, at most 7 minutes, at most 8 minutes, at most 9 minutes, at most 10 minutes, at most 20 minutes, at most 30 minutes, at most 40 minutes, at most 50 minutes, or at most 60 minutes. In some embodiments of any of the aspects, the lateral flow detection step is performed in at least 5 minutes. As a non-limiting example, the lateral flow detection step can be for a period of 30 minutes or less, 25 minutes or less, 20 minutes or less, 15 minutes or less, 10 minutes or less, or 5 minutes or less. In some embodiments of any of the aspects, the lateral flow detection step is performed in at most 5 minutes. As a non-limiting example, the lateral flow detection step is performed in at most 5 minutes, at most 6 minutes, at most 7 minutes, at most 8 minutes, at most 9 minutes, at most 10 minutes, at most 15 minutes, at most 20 minutes, at most 25 minutes, at most 30 minutes, at most 40 minutes, at most 50 minutes, at most 60 minutes, at most 70 minutes, at most 80 minutes, at most 90 minutes, or at most 100 minutes.

In some embodiments, the step of detecting the complex comprises a lateral flow assay. For example, the step of detecting the complex comprises: contacting the complex with a lateral flow device. The lateral flow device comprises a capture/test region comprising a capture probe immobilized thereon, wherein the capture probe is capable of binding with capture ligand. After the complex is captured, a detectable signal produced by the reporter molecule in the complex captured by the capture probe is detected.

In some embodiments, the step of detecting the complex comprises a micro-array assay. For example, the step of detecting the complex comprises: contacting the complex with a micro-array plate. The micro-array plate comprises a capture/test region comprising a capture probe immobilized thereon, wherein the capture probe is capable of binding with capture ligand. After the complex is captured, a detectable signal produced by the reporter molecule in the complex captured by the capture probe is detected.

In some embodiments of any of the aspects, a detectable signal from the reporter molecule is detected using colorimetric assays. Colorimetric assays use reagents that undergo a measurable color change in the presence of the reporter molecule. For example, para-Nitrophenylphosphate is converted into a yellow product by alkaline phosphatase enzyme. Coomassie Blue once bound to proteins elicits a spectrum shift, allowing quantitative dosage. A similar colorimetric assay, the Bicinchoninic acid assay, uses a chemical reaction to determine protein concentration. Enzyme linked immunoassays use enzyme-complexed-antibodies to detect antigens. Binding of the antibody is often inferred from the color change of reagents such as TMB. A colorimetric assay can be detected using a colorimeter, which is a device used to test the concentration of a solution by measuring its absorbance of a specific wavelength of light.

In some embodiments of any of the aspects, the colorimetric assay comprises nanoparticles whose optical properties change based on the particle density, e.g., plasmonic nanoparticles. In some embodiments of any of the aspects, the colorimetric assay produces a color change via change of pH in a minimally buffered reaction. In some embodiments of any of the aspects, the colorimetric assay produces a color change via oxidation/reduction of a substrate (e.g., ABTS (2,2′-Azinobis [3-ethylbenzothiazoline-6-sulfonic acid]-diammonium salt) through assembly of split Horseradish Peroxidase (HRP). In some embodiments of any of the aspects, the colorimetric assay produces a color change via assembly of an enzyme or protein with optical properties (e.g., split luciferase or split GFP equivalents). In some embodiments of any of the aspects, the colorimetric assay produces a color change via DNA-intercalating dyes, e.g., cyanine dyes, TOTO, TO-PRO, SYTOX, ethidium bromide, propidium iodide, DAPI, Hoechst dye, acridine orange, 7-AAD, LDS 751, and hydroxystilbamidine.

### Target Nucleic Acid

Described herein are methods, compositions, kits, and systems that can be used to detect a target nucleic acid. Without limitations, the target nucleic acid can be DNA, RNA or a mix or a DNA/RNA mixture. Accordingly, in some embodiments, the target nucleic acid is DNA. In some embodiments, the target nucleic acid is RNA. In some embodiments, the target nucleic acid is a cDNA. Further, the target nucleic acid can be single-stranded, double-stranded or partially double-stranded.

In some embodiments of the various aspects described herein, the method comprises amplifying the target nucleic acid to produce an amplicon. It is noted that the amplicon can be single-stranded, double-stranded or partially double-stranded. In some embodiments, the amplicon is single-stranded. In some other embodiments, the amplicon is double-stranded.

The methods, compositions, kits and systems provided herein can be used to detect, e.g., disease biomarkers, microbial nucleic acid sequences, viral nucleic acid sequences, and the like. In some embodiments, the methods and compositions provided herein can be used to diagnose, prevent, or treat a disease (e.g., an infection). In some embodiments, the methods, compositions, and kits provided herein can be used to identify the presence of SAR-CoV2 in a sample. In some embodiments, the methods, compositions, and kits provided herein can be used to diagnose a subject with an infection. In some embodiments, the infection is COVID19.

### Quencher Molecules

Embodiments of the various aspects described herein include a quencher molecule. As used herein, a “quencher” or “quencher molecule” refers to a molecule, composition or moiety capable of quenching a detectable label from a reporter molecule. Exemplary quencher molecules include, but are not limited to, Deep Dark Quenchers (Eurogentec), DABCYL, TAN/IRA, BHQ quenchers (e.g., BHQ-0, BHQ-1, BHQ-2, BHQ-3, and BHQ-10), BBQ®-650, ECLIPSE, Iowa Black® quenchers, QSY (e.g., QSY 21, QSY 15, QSY 7, and QSY 9), and IRDye® QC-1.

In some embodiments of any of the aspects, the quencher molecule quenches the specific wavelength of the fluorescence emitted by the reporter molecule. As a non-limiting example, some fluorophores, such as TET, HEX, and FAM, with an emission range between 500 nm to 550 nm are quenched by quenchers, such as Black hole quencher 1 (BHQ1) and Dabcyl, with an absorption range of 450 nm to 550 nm. Similarly, TMR, Texas red, ROX, Cy3, and Cy5 are quenched by BHQ2. See e.g., Marras, Selection of fluorophore and quencher pairs for fluorescent nucleic acid hybridization probes, Methods Mol Biol. 2006; 335:3-16; the content of which is incorporated herein by reference in its entirety.

In some embodiments of any of the aspects, the quencher molecule is a dark quencher. A dark quencher (also known as a dark sucker) is a substance that absorbs excitation energy from a reporter molecule, e.g., a fluorophore, and dissipates the energy as heat; while a typical (fluorescent) quencher re-emits much of this energy as light. Non-limiting examples of quencher molecules (e.g., non-fluorescent or dark quenchers that dissipate energy absorbed from a fluorescent dye) include the Black Hole Quenchers™ (Biosearch Technologies™); Iowa Black quenchers (e.g., Iowa Black FQ™ (“3IABkFQ”) and Iowa Black RQ™ (e.g., “3IAbRQSp”)); Eclipse® Dark Quenchers (Epoch Biosciences™), Zen™ quenchers (Integrated DNA Technologies™; “e.g., “ZEN”); TAO™ quenchers (Integrated DNA Technologies™; “e.g., “TAO”); Dabcyl (4-(4′-dimethylaminophenylazo)benzoic acid); Qxl™ quenchers; QSY® quenchers; and IRDye® QC-1. Additional non-limiting examples of quenchers are also provided in U.S. Pat. Nos. 6,465,175, 7,439,341, Ser. No. 12/252,721, U.S. Pat. No. 7,803,536, Ser. No. 12/853,755, U.S. Pat. Nos. 7,476,735, 7,605,243, 7,645,872, 8,030,460, Ser. No. 13/224,571, U.S. Pat. No. 8,916,345, the contents of each of which are incorporated herein by reference in their entireties.

In some embodiments of any of the aspects, the quencher molecule is an Iowa Black® quencher. In some embodiments of any of the aspects, the Iowa Black® quencher is preferably at the 5′ or 3′ position of the nucleic acid probe. In some embodiments of any of the aspects, the quencher molecule is Iowa Black® FQ, which has a broad absorbance spectra ranging from 420 to 620 nm with peak absorbance at 531 nm (i.e., the green-yellow region of the visible light spectrum). In some embodiments, Iowa Black® FQ (e.g., “3IABkFQ”) is used to quench fluorescein or other fluorescent dyes that emit in the green to pink spectral range. In some embodiments of any of the aspects, the quencher molecule is Iowa Black® RQ, which has a broad absorbance spectra ranging from 500 to 700 nm with peak absorbance at 656 nm (i.e., the orange-red region of the visible light spectrum). In some embodiments, Iowa Black® RQ (e.g., “3IAbRQSp”) is used to quench Texas Red®, Cy5, or other fluorescent dyes that emit in the red spectral range.

In some embodiments of any of the aspects, the quencher molecule is a ZEN quencher. In some embodiments of any of the aspects, the ZEN quencher is preferably at an internal position of the nucleic acid probe. See e.g., Lennox et al., Mol Ther Nucleic Acids. 2013 August; 2(8): e117; U.S. Pat. Nos. 8,916,345, 9,506,059; the contents of each of which are incorporated herein by reference in their entireties. ZEN can quench a similar range of fluorophores as Iowa Black® FQ, e.g., FAM, SUN, JOE, HEX, or MAX. In some embodiments, the nucleic acid probe comprises ZEN, Iowa Black® FQ, and a reporter molecule such as FAM.

In some embodiments of any of the aspects, the quencher molecule is a TAO quencher. In some embodiments of any of the aspects, the TAO quencher is preferably at an internal position of the nucleic acid probe. TAO can quench a similar range of fluorophores as Iowa Black® RQ, e.g., Cy3, ATTO550, ROX, Texas red, ATTO647N, or Cy5. In some embodiments, the nucleic acid probe comprises TAO, Iowa Black® RQ, and a reporter molecule, such as Cy5.

In some embodiments of any of the aspects, the quencher molecule is a black hole quencher. The Black Hole Quenchers™ are structures comprising at least three radicals selected from substituted or unsubstituted aryl or heteroaryl compounds, or combinations thereof, wherein at least two of the residues are linked via an exocyclic diazo bond (see, e.g., International Publication No. WO2001086001). Black Hole Quenchers (BHQ) are capable of quenching across the entire visible spectrum. Non-limiting examples of Black Hole Quenchers include BHQ-0 (430-520 nm); BHQ-1 (480-580 nm, 534 nm absorbance (abs) max); BHQ-2 (520-650 nm, 544 nm abs max); BHQ-3 (620-730 nm, 672 nm abs max); and BHQ-10 (480-550 nm, 516 nm abs max; Water Soluble).

In some embodiments of any of the aspects, the quencher molecule is Dabcyl (4-(4′-dimethylaminophenylazo)benzoic acid) or a derivative thereof. Dabcyl absorbs in the green region of the visible light spectrum (e.g., 346-489 nm, with a peak absorbance at 474 nm) and can be used with fluorescein or other fluorophores that emit in the green region.

In some embodiments of any of the aspects, the quencher molecule is an Eclipse® Dark Quencher. The absorbance maximum for the Eclipse Quencher is at 522 nm, compared to 479 nm for Dabcyl. In addition, the structure of the Eclipse Quencher is substantially more electron deficient than that of Dabcyl and this leads to better quenching over a wider range of dyes, especially those with emission maxima at longer wavelengths (red shifted) such as Redmond Red and Cyanine 5. In addition, with an absorption range from 390 nm to 625 nm, the Eclipse Quencher is capable of effective quenching of a wide range of fluorophores.

In some embodiments of any of the aspects, the quencher molecule is a QSY® quencher. Non-limiting examples of QSY quenchers include QSY35 (410-500 nm, 475 nm max abs), QSY7 (500-600 nm, 560 nm max abs), QSY21 (590-720 nm, 661 nm abs max), and QSY9 (500-600 nm, 562 nm abs max).

In some embodiments of any of the aspects, the quencher molecule is a Qxl™ quencher. Qxl™ quenchers span the full visible spectrum. Non-limiting examples of QXL quenchers include QXL490 (495 nm abs max, can be used as a quencher for EDANS, AMCA, and most coumarin fluorophores), QXL520 (— 520 nm abs max, can be used as a quencher for FAM), QXL570 (578 nm abs max, can be used as a quencher for rhodamines (such as TAMRA, sulforhodamine B, ROX) and Cy3 fluorophores), QXL610 (˜610 nm abs max, can be used as a quencher for ROX), and QXL670 (668 nm abs max, can be used as a quencher for Cy5 and Cy5-like fluorophores such as HiLyte™ Fluor 647).

In some embodiments of any of the aspects, the quencher molecule is IRDye QC-1. IRDye QC-1 quenches dyes from the visible to the near-infrared range (500-900 nm, max abs 737 nm).

In some embodiments, the reporter molecule and the quencher molecule are a FRET pair. In some cases, the FRET donor is Cy3 and the FRET acceptor is Cy5. Further non-limiting examples of FRET pairs include: Cy3 and MG; Cy3 and acetylenic MG; Cy3, Cy5 and MG; Cy3 and DIR; Cy3 and Cy5 and ICG; FITC and TRITC; EGFP and Cy3; CFP and YFP; and EGFP and YFP.

In embodiments of the various aspects described herein, the reporter molecule and the quencher molecule are positioned such that such that the quencher molecule quenches a detectable signal produced by the reporter molecule when the probe is not complexed with the target nucleic acid. Generally, the reporter molecule and the quencher molecule are separated by no more than 15 nucleotides. For example, the reporter molecule and the quencher molecule are separated by no more than 14 nucleotides, no more than 13 nucleotides, no more than 12 nucleotides, no more than 11 nucleotides, no more than 10 nucleotides, no more than 9 nucleotides, no more than 8 nucleotides, no more than 7 nucleotides, no more than 6 nucleotides, no more than 5 nucleotides, no more than 4 nucleotides, no more than 3 nucleotides, no more than 2 nucleotides or no more than 1 nucleotide. In some embodiments, the reporter molecule and the quencher molecule are next to each other. For example, if the reporter molecule and the quencher molecules are attached to separate strands, they are in a complementary position to each other.

In some embodiments of any one of the aspects described herein the nucleic acid strand of the probe is hybridized with a second strand, i.e., the nucleic acid of the probe comprises a first and second nucleic strands, where the first and second strands are at least partially hybridized to each other. In some further embodiments of these one of the first and second nucleic strand comprises a reporter molecule and the other of the first and second nucleic strand comprises a quencher molecule. In some embodiments, when the first and second strands are hybridized to each other, the quencher molecule quenches the detectable signal from the reporter molecule. For example, reporter molecule and the quencher molecule are positioned such that the quencher molecule quenches a detectable signal produced by the reporter molecule when the probe is not complexed with a target nucleic acid. When the probe is contacted with the target nucleic acid, one of the first or second strand is released either the reporter molecule or the quencher molecule is released, thereby unquenching the detectable signal of the reporter molecule to generate a detectable signal indicative of the target nucleic acid sequence.

It noted that a quencher molecule can be attached at any position of the nucleic acid strand it is attached to. For example, the quencher molecule can be attached at 5′-end of the nucleic acid strand it is attached to. Alternatively, the quencher molecule can be attached at 5′-end of the nucleic acid strand it is attached to. In yet another non-limiting alternative, the quencher molecule can be attached at an internal position of the nucleic acid strand it is attached to. In addition, the quencher molecule can be linked directly, i.e., via a bond or indirectly, e.g., via linker to the nucleic acid strand it is attached to.

In some embodiments of any one of the aspects described herein, the nucleic acid strand comprising the quencher molecule comprises two or more quencher molecules. When two or more quencher molecules are present in a nucleic acid strand, they can be the same or different. Further, they can be attached independently at any position of the nucleic acid strand. For example, in some embodiments, a nucleic acid strand comprises two quencher molecules, where one of the quencher molecules is attached at the 5′-end of the strand and the second capture is attached to the 3′-end. In some embodiments, a nucleic acid strand comprises two quencher molecules, where one quencher molecule is linked to the nucleic acid strand and the second quencher molecule is linked to the quencher molecule linked to the strand.

In some embodiments of any one of the aspects described herein the nucleic acid strand of the probe is hybridized with a second strand, i.e., the nucleic acid of the probe comprises a first and second nucleic strands, where the first and second strands are at least partially hybridized to each other, and wherein a reporter molecule is linked to one of the first or second nucleic acid strands of the probe and a quencher molecule is linked to the other of the first or second nucleic acid strands. In some further embodiments of this, the reporter molecule is linked to the 5′-end of the strand it is attached to and the quencher molecule is attached to the 3′-end of the strand it is attached to. In some other embodiments, the reporter molecule is linked to the 3′-end of the strand it is attached to and the quencher molecule is attached to the 5′-end of the strand it is attached to.

For clarity, it is noted that the quencher molecule can be linked to the nucleic acid strand of the probe that comprises the binding domain for the molecule capable of stabilizing or enhancing interactions between two nucleic acids, or the strand complementary to said strand.

In some embodiments of any of the aspects, the quenching is partial quenching or complete quenching. As used herein the term “completely quenched” refers to the inability to detect any signal from the reporter molecule, i.e., 100% quenched or 0% detectable signal (e.g., fluorescence). As used herein the term “partially quenched” refers to a detectable signal from the reporter molecule that is reduced compared to the full detectable signal from the reporter molecule. In some embodiments of any of the aspects, “partially quenched” refers to signal from the reporter molecule that is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.5%, at least 99.9% or more.

### Domains

In some embodiments of any one of the aspects described herein, each hybridization domain can be independently from about 5 nucleotides to about 100 nucleotides in length. For example, each domain can be independently from about 10 nucleotides to about 75 nucleotides in length. In some embodiments of any one of the aspects described herein, each domain can be independently from about 15 nucleotides to about 50 nucleotides in length. For example, each domain can be independently from about 20 nucleotides to about 40 nucleotides in length. In some embodiments of any one of the aspects described herein, each domain is independently 15, 16, 17, 18, 19, 20, 21, 22, 23, 24, 25, 26, 27, 28, 29, 30, 31, 32, 33, 34, 35, 36, 37, 38, 39 or 40 nucleotides in length.

In some embodiments of any one of the aspects described herein, each binding domain of the nucleic acid strand of the probe can be independently from about 5 nucleotides to about 100 nucleotides in length. For example, each binding domain can be independently from about 10 nucleotides to about 75 nucleotides in length. In some embodiments of any one of the aspects described herein, each binding domain can be independently from about 15 nucleotides to about 50 nucleotides in length. For example, each binding domain can be independently from about 20 nucleotides to about 40 nucleotides in length. In some embodiments of any one of the aspects described herein, each binding domain is independently 15, 16, 17, 18, 19, 20, 21, 22, 23, 24, 25, 26, 27, 28, 29, 30, 31, 32, 33, 34, 35, 36, 37, 38, 39 or 40 nucleotides in length.

In some embodiments of any one of the aspects described herein, each hybridization domain described herein can be independently from about 5 nucleotides to about 100 nucleotides in length. For example, each hybridization domain can be independently from about 10 nucleotides to about 75 nucleotides in length. In some embodiments of any one of the aspects described herein, each hybridization domain can be independently from about 15 nucleotides to about 50 nucleotides in length. For example, each hybridization domain can be independently from about 20 nucleotides to about 40 nucleotides in length. In some embodiments of any one of the aspects described herein, each hybridization domain is independently 15, 16, 17, 18, 19, 20, 21, 22, 23, 24, 25, 26, 27, 28, 29, 30, 31, 32, 33, 34, 35, 36, 37, 38, 39 or 40 nucleotides in length.

Each non-hybridization domain described herein can be independently from 1 nucleotide to about 50 nucleotides in length. For example, each non-hybridization domain can be independently from 1 nucleotide to about 25 nucleotides in length. In some embodiments of any one of the aspects described herein, each non-domain can be independently from 1 nucleotide to about 20 nucleotides in length. For example, each hybridization domain can be independently from 1 nucleotide to about 15 nucleotides in length. In some embodiments of any one of the aspects described herein, each hybridization domain is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides in length.

In some embodiments of any one of the aspects described, two domains in the same strand are separated by from 1 to 50 nucleotides. For example, two domains in the same strand are separated by from 1 to 25 nucleotides. In some embodiments of any one of the aspects described herein, two domains in the same strand can be separated by from 1 to 20 nucleotides. For example, two domains in the same strand can be separated by from 1 to 15 nucleotides. In some embodiments of any one of the aspects described herein, two domains in the same strand are separated by 1, 2, 3, 4, 5, 6, 7, 8, 9, 10, 11, 12, 13, 14, 15, 16, 17, 18, 19, 20, 21, 22, 23, 24, or 25 nucleotides.

### Compositions

In another aspect, provided herein are compositions useful in detecting a target nucleic acid. In some embodiments the composition comprises a probe and a target nucleic acid or an amplicon from amplification of the target nucleic acid, wherein the probe comprises a first nucleic acid strand and a molecule capable of stabilizing or enhancing interactions between two nucleic acids bound with the first nucleic acid strand, and wherein the first nucleic acid strand comprises a binding domain comprising a nucleotide sequence substantially complementary to at least a first portion of the target nucleic acid or the amplicon.

In some embodiments, the composition comprises a probe and a target nucleic acid or an amplicon from amplification of the target nucleic acid, wherein the probe comprises a first nucleic acid strand, a second nucleic acid strand, and a molecule capable of stabilizing or enhancing interactions between two nucleic acids, wherein the first nucleic acid strand comprises a binding domain comprising a nucleotide sequence substantially complementary to at least a first portion of the target nucleic acid or amplicon, wherein the first nucleic acid strand comprises a first hybridization domain linked with the binding domain and the second nucleic acid strand is hybridized with the first hybridization domain of the first nucleic acid strand, and wherein the first nucleic acid strand comprises a reporter molecule capable of producing a detectable signal and the second nucleic acid strand comprises a capture ligand. Optionally, the first nucleic acid strand and the second nucleic acid strand hybridized with each other to form a double-stranded structure comprising a single-stranded loop region, wherein the first nucleic acid strand comprises a first hybridization domain linked to the binding domain linked to a second hybridization domain.

In some embodiments of any one of the aspects, the composition comprises a first probe, a second probe, and a target nucleic acid or an amplicon from amplification of the target nucleic acid, wherein the first probe comprises a first nucleic acid strand and a first molecule capable of stabilizing or enhancing interactions between two nucleic acids, and wherein the first nucleic acid strand of the first probe comprises a binding domain comprising a nucleotide sequence substantially complementary to at least a first portion of the target nucleic acid or amplicon. Optionally, the second probe comprises a first nucleic acid strand and a first molecule capable of stabilizing or enhancing interactions between two nucleic acids, and wherein the first nucleic acid strand of the second probe comprises a binding domain comprising a nucleotide sequence substantially complementary to at least a second portion of the target nucleic acid or the amplicon, and wherein one of the first nucleic acid strand of the first probe and the first nucleic acid strand of the second probe comprises a reporter molecule capable of producing a detectable signal and the other of the first nucleic acid strand of the first probe and the first nucleic acid strand of the second probe comprises a capture ligand.

In some embodiments, the composition comprises a probe and a target nucleic acid or an amplicon from amplification of a target nucleic acid, wherein the probe comprises a first nucleic acid strand, a second nucleic acid strand, and a first molecule capable of stabilizing or enhancing interactions between two nucleic acids, and wherein the first nucleic acid strand of the first probe comprises a binding domain comprising a nucleotide sequence substantially complementary to at least a first portion of the target nucleic acid or the amplicon, wherein the first nucleic acid strand comprises a first hybridization domain linked with the binding domain and the second nucleic acid strand is hybridized with the first hybridization domain of the first nucleic acid strand. Optionally, one of the first and second nucleic strand comprises a reporter molecule capable of producing a detectable signal and the other of the first and second nucleic strand comprises a quencher molecule, and wherein the quencher molecule quenches the detectable signal from the reporter molecule when the first and second nucleic acid strands are hybridized with each other, wherein the first hybridization domain and the binding domain of the first nucleic acid strand together comprise a nucleotide sequence substantially complementary to at least a portion of the amplicon.

In some embodiments of any of the aspects, the composition further comprising reagents for preparing an amplicon from the target nucleic acid. For example, the composition further comprises one or more of a DNA polymerase or reverse transcriptase, RNAse H, dNTPs, and buffers. In some embodiments, the composition further comprises means for detecting a detectable signal from the reporter molecule.

In some embodiments, the composition is on a surface of a lateral flow device or micro-array plate.

### Kits

Another aspect of the technology described herein relates to kits for detecting a target nucleic acid. Described herein are kit components that can be included in one or more of the kits described herein. The kit can comprise any of the compositions provided herein and packaging and materials therefore. Accordingly, in some embodiments the kit comprises a primer set for preparing an amplicon from a target nucleic acid, and a first probe, wherein the first probe comprises a first nucleic acid strand and a molecule capable of stabilizing or enhancing interactions between two nucleic acids bound with the first nucleic acid strand, wherein the first nucleic acid strand comprises a binding domain comprising a nucleotide sequence substantially complementary to at least a first portion of the amplicon prepared from a target nucleic acid using the primer set. In some embodiments, the first nucleic acid strand comprises a reporter molecule capable of producing a detectable signal and at least one primer in the primer set comprises a capture ligand. In some other embodiments, the first nucleic acid strand comprises capture ligand and at least one primer in the primer set comprises a reporter molecule capable of producing a detectable signal. In some embodiments, the first nucleic acid strand comprises a first hybridization domain linked with the binding domain and the first probe further comprises a second nucleic acid strand hybridized with the first hybridization domain of the first nucleic acid strand.

In some embodiments, the kit further comprises a second probe, wherein the second probe comprises a first nucleic acid strand and a first molecule capable of stabilizing or enhancing interactions between two nucleic acids, and wherein the first nucleic acid strand of the second probe comprises a binding domain comprising a nucleotide sequence substantially complementary to at least a second portion of the amplicon prepared from a target nucleic acid using the primer set. In some embodiments, one of the first nucleic acid strand of the first probe and the first nucleic acid strand of the second probe comprises a reporter molecule capable of producing a detectable signal and the other of the first nucleic acid strand of the first probe and the first nucleic acid strand of the second probe comprises a capture ligand.

In some embodiments, the kit comprises a primer set for preparing an amplicon from a target nucleic acid, and a first probe, wherein the first probe comprises a first nucleic acid strand, a second nucleic acid strand, and a molecule capable of stabilizing or enhancing interactions between two nucleic acids. In some embodiments, the first nucleic acid strand comprises a first hybridization domain linked with a binding domain and/or the second nucleic acid strand is hybridized with the first hybridization domain of the first nucleic acid strand. In some embodiments, one of the first and second nucleic strand comprises a reporter molecule capable of producing a detectable signal and the other of the first and second nucleic strand comprises a quencher molecule, and wherein the quencher molecule quenches the detectable signal from the reporter molecule when the first and second nucleic acid strands are hybridized with each other.

Generally, the kit comprises an effective amount of the reagents as described herein. As will be appreciated by one of skill in the art, the reagents can be supplied in a lyophilized form or a concentrated form that can be diluted or suspended in liquid prior to use. The kit reagents described herein can be supplied in aliquots or in unit doses.

In some embodiments, the components described herein can be provided singularly or in any combination as a kit. Such a kit includes the components described herein and packaging materials thereof. In addition, a kit optionally comprises informational material.

In some embodiments, the compositions in a kit can be provided in a watertight or gas tight container which in some embodiments is substantially free of other components of the kit. For example, the reagents described herein can be supplied in more than one container, e.g., it can be supplied in a container having sufficient reagent for a predetermined number of applications, e.g., 1, 2, 3 or greater. One or more components as described herein can be provided in any form, e.g., liquid, dried or lyophilized form. Liquids or components for suspension or solution of the reagents can be provided in sterile form and should not contain microorganisms or other contaminants. When the components described herein are provided in a liquid solution, the liquid solution preferably is an aqueous solution.

The informational material can be descriptive, instructional, marketing or other material that relates to the methods described herein. The informational material of the kits is not limited in its form. In some embodiments, the informational material can include information about production of the reagents, concentration, date of expiration, batch or production site information, and so forth. In some embodiments, the informational material relates to methods for using or administering the components of the kit.

The kit will typically be provided with its various elements included in one package, e.g., a fiber-based, e.g., a cardboard, or polymeric, e.g., a Styrofoam box. The enclosure can be configured so as to maintain a temperature differential between the interior and the exterior, e.g., it can provide insulating properties to keep the reagents at a preselected temperature for a preselected time.

In some embodiments of any of the aspects, the kit can further comprise a detection device. As a non-limiting example, a detection device can be a lateral flow device or a micro-plate. In some embodiments of any of the aspects, the kit and/or the detection device is field-deployable, i.e., transportable, non-refrigerated, and/or inexpensive. In some embodiments of any of the aspects, a detection device further comprises a wireless device (e.g., a cell phone, a personal digital assistant (PDA), a tablet).

Some embodiments of the technology described herein can be defined according to any of the following numbered paragraphs:
Paragraph 1: A method for detecting a target nucleic acid, the method comprising:
   a. contacting a double-stranded or single-stranded amplicon from amplification of a target nucleic acid with a first probe to form a complex comprising the first probe and the amplicon, wherein the first probe comprises a first nucleic acid strand and a molecule bound with the first nucleic acid strand, wherein the molecule is capable of localizing a single-stranded nucleic acid to a double-stranded nucleic acid or enhancing the kinetics of hybridization between two single-strand nucleic acids, and wherein the first nucleic acid strand comprises a binding domain comprising a nucleotide sequence substantially complementary to at least a first portion of the amplicon; and
   b. detecting the complex comprising the first probe and the amplicon from step (a).
Paragraph 2: The method of paragraph 1, wherein the molecule capable of localizing a single-stranded nucleic acid to a double-stranded nucleic acid or enhancing the kinetics of hybridization between two single-stranded nucleic acids is selected from the group consisting of recombinases, CRISPR-Cas proteins, single-stranded binding proteins, zinc finger nucleases, transcription activator-like effector nucleases (TALEN), site-specific recombinases, transcription factors, and any combinations thereof.
Paragraph 3: The method of paragraph 1, wherein the molecule capable of localizing a single-strand nucleic acid strand to a double-stranded nucleic acid or enhancing the kinetics of hybridization between two single-stranded nucleic acids is a recombinase or CRISPR-Cas protein, optionally, the molecule capable of localizing a single-strand nucleic acid strand to a double-stranded nucleic acid lacks nuclease activity.
Paragraph 4: The method of claim any one of paragraphs 1-3, wherein one of the amplicon and the first nucleic acid strand comprises a reporter molecule capable of producing a detectable signal and the other of the amplicon and the first nucleic acid strand comprises a capture ligand, and wherein said step of detecting the complex comprises detecting the reporter molecule in the complex.
Paragraph 5: The method of paragraph 4, wherein the first nucleic acid strand comprises a first hybridization domain linked with the binding domain and the first probe comprises a second nucleic acid strand hybridized with the first hybridization domain of the first nucleic acid strand.
Paragraph 6: The method of paragraph 5, wherein the first hybridization domain is linked to the 5′-end of the binding domain.
Paragraph 7: The method of paragraph 5, wherein the first hybridization domain is linked to the 3′-end of the binding domain.
Paragraph 8: The method of any one of paragraphs 4-7, wherein the first nucleic acid strand comprises a first hybridization domain linked with the binding domain linked with a second hybridization domain, and the first probe further comprises a second nucleic acid strand hybridized with the first nucleic acid strand,
   wherein the second nucleic acid strand comprises a first hybridization domain linked with a non-hybridizing domain linked with a second hybridization domain,
   wherein the first hybridization domain of the first nucleic acid strand and the second hybridization domain of the second nucleic acid strand are hybridized with each other forming a first double-stranded region,
   wherein the second hybridization domain of the first nucleic acid strand and the first hybridization domain of the second nucleic acid strand are hybridized with each other forming a second double-stranded region,
   wherein the binding domain of the first nucleic acid strand and the non-hybridizing domain of the second nucleic acid strand do not hybridize to each other, and
   wherein one of the amplicon and the first nucleic acid strand comprises a reporter molecule capable of producing a detectable signal and the other of the amplicon and the first nucleic acid strand comprises a capture ligand, and
   wherein said step of detecting the complex comprises detecting the reporter molecule in the complex.
Paragraph 9: The method of any one of paragraphs 4-8, wherein the first nucleic acid strand comprises the reporter molecule and the amplicon comprises the capture ligand.
Paragraph 10: The method of paragraph 9, wherein the capture ligand is attached to the 5′-end of the nucleic acid strand it is attached to.
Paragraph 11: The method of paragraph 9, wherein the capture ligand is attached to the 3′-end of the nucleic acid strand it is attached to.
Paragraph 12: The method of paragraph 9, wherein the capture ligand is at an internal position of the nucleic acid strand it is attached to.
Paragraph 13: The method of any one of paragraphs 10-11, wherein the amplicon comprises at least one second capture ligand.
Paragraph 14: The method of paragraph 13, wherein one capture ligand is at the 5′-end and one capture ligand is the 3′-end of the nucleic acid strand they are attached to.
Paragraph 15: The method of paragraph 13, wherein one capture ligand is at the 5′-end and one capture ligand is at an internal position of the nucleic acid strand they are attached to.
Paragraph 16: The method of paragraph 13, wherein one capture ligand is at the 3′-end and one capture ligand is at an internal position of the nucleic acid strand they are attached to.
Paragraph 17: The method of paragraph 13, wherein one capture ligand is at the 5′-end, one capture ligand is at the 3′-end, and one capture ligand is at an internal position of the nucleic acid strand they are attached to.
Paragraph 18: The method of any one of paragraphs 4-7, wherein the amplicon comprises the reporter molecule and the first nucleic acid strand comprises the capture ligand.
Paragraph 19: The method of paragraph 18, wherein the amplicon further comprises a second reporter molecule.
Paragraph 20: The method of any one of paragraphs 18-19, wherein the capture ligand is attached to the 5′-end of the nucleic acid strand it is attached to.
Paragraph 21: The method of any one of paragraphs 18-19, wherein the capture ligand is attached to the 3′-end of the nucleic acid strand it is attached to.
Paragraph 22: The method of any one of paragraphs 18-19, wherein the capture ligand is at an internal position of the nucleic acid strand it is attached to.
Paragraph 23: The method of any one of paragraphs 18-22, wherein the amplicon comprises at least one second capture ligand.
Paragraph 24: The method of paragraph 23, wherein one capture ligand is at the 5′-end and one capture ligand is the 3′-end of the nucleic acid strand they are attached to.
Paragraph 25: The method of paragraph 23, wherein one capture ligand is at the 5′-end and one capture ligand is at an internal position of the nucleic acid strand they are attached to.
Paragraph 26: The method of paragraph 23, wherein one capture ligand is at the 3′-end and one capture ligand is at an internal position of the nucleic acid strand they are attached to.
Paragraph 27: The method of paragraph 23, wherein one capture ligand is at the 5′-end, one capture ligand is at the 3′-end, and one capture ligand is at an internal position of the nucleic acid strand they are attached to.
Paragraph 28: The method of any one of paragraphs 1-3, wherein the first probe comprises a second nucleic acid strand hybridized with the first strand and forming a double-stranded structure comprising a single-stranded loop region,
   wherein the first nucleic acid strand comprises a first hybridization domain linked to the binding domain linked to a second hybridization domain,
   wherein the second nucleic acid strand comprises a first hybridization domain linked to a non-hybridizing domain linked to a second hybridization domain,
   wherein the first hybridization domain of the first nucleic acid strand and the second hybridization domain of the second nucleic acid strand are hybridized with each other forming a first double-stranded region,
   wherein the second hybridization domain of the first nucleic acid strand and the first hybridization domain of the second nucleic acid strand are hybridized with each other forming a second double-stranded region,
   wherein the binding domain of the first nucleic acid strand and the non-hybridizing domain of the second nucleic acid strand do not hybridize to each other,
   wherein the first nucleic acid strand comprises a reporter molecule capable of producing a detectable signal and the second nucleic acid strand comprises a capture ligand, and where absence of a detectable signal from the reporter molecule indicates presence of the target nucleic acid.
Paragraph 29: The method of any one of paragraph 1-3, wherein the method comprises contacting the amplicon with the first probe and a second probe to form a complex comprising the first probe, the second probe and the amplicon, and
   wherein the second probe comprises a first nucleic acid strand (first nucleic acid of the second probe) and a molecule bound with the first nucleic acid strand of the second probe and capable of localizing a single-strand nucleic acid strand to a double-stranded nucleic acid or enhancing the kinetics of hybridization between two single-stranded nucleic acids, and wherein the first nucleic acid strand of the second probes comprises a binding domain comprising a nucleotide sequence substantially complementary to at least a second portion of the amplicon,
   wherein one of the first nucleic acid strand of the first probe and the first nucleic acid strand of the second probe comprises a reporter molecule capable of producing a detectable signal and the other of the first nucleic acid strand of the first probe and the first nucleic acid strand of the second probe comprises a capture ligand, and
   wherein said step of detecting the complex comprises detecting the reporter molecule in the complex.
Paragraph 30: The method of any one of paragraphs 1-29, wherein detecting the reporter molecule comprises detecting a detectable signal produced by the reporter molecule.
Paragraph 31: The method of any one of paragraphs 1-30, wherein said step of detecting the complex comprises fluorescence detection, luminescence detection, chemiluminescence detection, colorimetric detection, immunofluorescence detection, or electrochemical detection.
Paragraph 32: The method of any one of paragraphs 1-31, wherein said step of detecting the complex comprises a lateral flow assay.
Paragraph 33: The method of any one of paragraphs 1-32, wherein said step of detecting the complex comprises:
   a. contacting the complex with a lateral flow device, wherein the lateral flow device or the micro-array plate comprises a capture/test region comprising a capture probe immobilized thereon, wherein the capture probe is capable of binding with capture label; and
   b. detecting the reporter molecule in the complex captured by the capture probe.
Paragraph 34: The method of any one of paragraphs 1-31, wherein said step of detecting the complex comprises micro-array detection.
Paragraph 35: The method of paragraph 34, wherein said step of detecting the complex comprises:
   a. contacting the complex with a micro-array plate, wherein the micro-array plate comprises a capture/test region comprising a capture probe immobilized thereon and capable of binding with capture label; and
   b. detecting the reporter molecule in the complex captured by the capture probe.
Paragraph 36: The method of any one of paragraphs 1-3, wherein the first nucleic acid strand of the first probe comprises a reporter molecule capable of producing a detectable signal, and wherein said step of detecting the complex comprises:
   a. contacting the complex with a lateral flow device or a micro-array plate, wherein the lateral flow device or the micro-array plate comprises a capture/test region comprising a capture nucleic acid strand immobilized thereon, wherein the capture nucleic acid strand is bound with a molecule capable of localizing a single-strand nucleic acid strand to a double-stranded nucleic acid or enhancing the kinetics of hybridization between two single-stranded nucleic acids, and wherein the capture nucleic acid strand comprises a nucleotide sequence substantially complementary to at least a second portion of the amplicon; and
   b. detecting the reporter molecule in the complex captured by the capture probe.
Paragraph 37: The method of paragraph 36, wherein the method comprises immobilizing the capture nucleic acid strand on the capture/test region prior to contacting with the complex.
Paragraph 38: The method of paragraph 36 or 37, wherein the capture nucleic acid comprises a capture ligand conjugated thereto and the capture/test region comprises a capture probe immobilized thereon, and wherein the capture probe is capable of binding with capture ligand of the capture nucleic acid strand.
Paragraph 39: The method of any one of paragraphs 36-28, wherein the capture nucleic acid comprises a molecule capable of localizing a single-strand nucleic acid strand to a double-stranded nucleic acid
Paragraph 40: The method of any one of paragraphs 29-39, wherein the first and second portion of the amplicon are separated from each other by at least 5 nucleotides or at least 5 nucleotide base-pairs.
Paragraph 41: The method of any one of paragraphs 29-40, wherein the one of the first and second portion of the amplicon is on a first strand of the amplicon and the other of the first and second portion of the amplicon is on a second strand of the amplicon.
Paragraph 42: The method of any one of paragraphs 29-40, wherein the first and second portion of the amplicons are same strand of the amplicon.
Paragraph 43: The method of any one of paragraphs 29-42, wherein the capture ligand is linked at the 5′-end of the nucleic acid strand it is attached to.
Paragraph 44: The method of any one of paragraphs 29-42, wherein the capture ligand is linked at the 5′-end of the nucleic acid strand it is attached to.
Paragraph 45: The method of any one of paragraphs 29-42, wherein the capture ligand is at an internal position of the nucleic acid strand it is attached to.
Paragraph 46: The method of any one of paragraphs 29-45, wherein the amplicon comprises at least one second capture ligand.
Paragraph 47: The method of paragraph 46, wherein one capture ligand is at the 5′-end and one capture ligand is the 3′-end of the nucleic acid strand they are attached to.
Paragraph 48: The method of paragraph 46, wherein one capture ligand is at the 5′-end and one capture ligand is at an internal position of the nucleic acid strand they are attached to.
Paragraph 49: The method of paragraph 46, wherein one capture ligand is at the 3′-end and one capture ligand is at an internal position of the nucleic acid strand they are attached to.
Paragraph 50: The method of paragraph 46, wherein one capture ligand is at the 5′-end, one capture ligand is at the 3′-end, and one capture ligand is at an internal position of the nucleic acid strand they are attached to.
Paragraph 51: The method of any one of paragraphs 1-3, wherein the first nucleic acid strand comprises a first hybridization domain linked to the binding domain, and the first probe comprises a second nucleic acid strand hybridized with the hybridization domain of the first strand, wherein one of the first and second nucleic strand comprises a reporter molecule capable of producing a detectable signal and the other of the first and second nucleic strand comprises a quencher molecule, and wherein the quencher molecule quenches the detectable signal from the reporter molecule when the first and second nucleic acid strands are hybridized with each other,
wherein the first hybridization domain and the binding domain together comprise a nucleotide sequence substantially complementary to at least a portion of the amplicon, and
wherein said step of detecting the complex comprises detecting a detectable signal produced by the reporter molecule.
Paragraph 52: The method of paragraph 51, wherein the first hybridization domain is linked to the 5′-end of the binding domain.
Paragraph 53: The method of paragraph 51, wherein the first hybridization domain is linked to the 3′-end of the binding domain.
Paragraph 54: The method of any one of paragraphs 1-3, wherein the first nucleic acid strand comprises a first hybridization domain linked with the binding domain linked with a second hybridization domain, and the first probe comprises a second nucleic acid strand hybridized with the first nucleic acid strand,
   wherein the second nucleic acid strand comprises a first hybridization domain linked with a non-hybridizing domain linked with a second hybridization domain,
   wherein the first hybridization domain of the first nucleic acid strand and the second hybridization domain of the second nucleic acid strand are hybridized with each other forming a first double-stranded region,
   wherein the second hybridization domain of the first nucleic acid strand and the first hybridization domain of the second nucleic acid strand are hybridized with each other forming a second double-stranded region,
   wherein the binding domain of the first nucleic acid strand and the non-hybridizing domain of the second nucleic acid strand do not hybridize to each other,
   wherein one of the first and second nucleic strand comprises a reporter molecule capable of producing a detectable signal and the other of the first and second nucleic strand comprises a quencher molecule, and wherein the quencher molecule quenches the detectable signal from the reporter molecule when the first and second nucleic acid strands are hybridized with each other, and
   wherein said step of detecting the complex comprises detecting a detectable signal produced by the reporter molecule.
Paragraph 55: The method of any one of paragraphs 51-54, wherein the reporter molecule and the quencher molecule are a FRET pair.
Paragraph 56: The method of any one of paragraphs 51-55, wherein the first nucleic acid strand comprises the reporter molecule.
Paragraph 57: The method of any one of paragraphs 51-55, wherein the second nucleic acid strand comprises the reporter molecule.
Paragraph 58: The method of any one of paragraphs 51-57, wherein the reporter molecule is attached to the 5′-end of the nucleic acid strand it is attached to.
Paragraph 59: The method of any one of paragraphs 51-57, wherein the reporter molecule is attached to the 3′-end of the nucleic acid strand it is attached to.
Paragraph 60: The method of any one of paragraphs 51-58, wherein the quencher molecule is attached to the 5′-end of the nucleic acid strand it is attached to.
Paragraph 61: The method of any one of paragraphs 51-58, wherein the quencher molecule is attached to the 3′-end of the nucleic acid strand it is attached to.
Paragraph 62: The method of any one of paragraphs 51-61, wherein said detecting the complex comprises fluorescence detection, luminescence detection, chemiluminescence detection, colorimetric detection, immunofluorescence detection, or electrochemical detection.
Paragraph 63: The method of any one of paragraphs 4-62, wherein the molecule capable of localizing a single-strand nucleic acid strand to a double-stranded nucleic acid is a recombinase.
Paragraph 64: The method of paragraph 63, wherein the recombinase is selected from the group consisting of RecA, UvsX, RadA, Rad51, Dmcl, UvsY, Cre, Flp, Dre, SCre, VCre, Vika, B2, B3, KD, ΦC31, Bxb1, λ,HK022, HP1, γδ, ParA, Tn3, Gin, R4, TP901-1, TG1, PhiRv1, PhiBT1, SprA, XisF, TnpX, R, A118, spoIVCA, PhiMR11, SCCmec, TndX, XerC, XerD, XisA, Hin, Cin, mrpA, beta, PhiFC1, Fre, Clp, sTre, FimE, HbiFm, and homologues thereof, and modified versions thereof.
Paragraph 65: The method of paragraph 64, wherein the recombinase is RecA.
Paragraph 66: The method of any one of paragraphs 1-3, wherein the first nucleic acid strand comprises a first hybridization domain linked to the binding domain, and the first probe further comprises a second nucleic acid strand hybridized with the hybridization domain of the first strand,
   wherein the first or second nucleic strand comprises a capture ligand, and
   wherein the amplicon comprises a reporter molecule capable of producing a detectable signal, and
   wherein said step of detecting the complex comprises:
      a. contacting the complex with a lateral flow device or micro-array plate, wherein the lateral flow device or the micro-array plate comprises a capture/test region comprising a capture probe immobilized thereon, wherein the capture probe is capable of binding with capture ligand; and
      b. detecting the reporter molecule in a complex captured by the capture probe.
Paragraph 67: The method of paragraph 66, wherein the first hybridization domain is linked to the 5′-end of the binding domain.
Paragraph 68: The method of paragraph 66, wherein the first hybridization domain is linked to the 3′-end of the binding domain.
Paragraph 69: The method of any one of paragraphs 66-68, wherein the first nucleic acid strand comprises the capture ligand.
Paragraph 70: The method of any one of paragraphs 66-68, wherein the second nucleic acid strand comprises the capture ligand.
Paragraph 71: The method of any one of paragraphs 66-70, wherein the capture ligand is attached to the 5′-end of the nucleic acid strand it is attached to.
Paragraph 72: The method of any one of paragraphs 66-71, wherein the capture ligand is attached to the 3′-end of the nucleic acid strand it is attached to.
Paragraph 73: The method of any one of paragraphs 66-72, wherein the molecule capable of localizing a single-strand nucleic acid strand to a double-stranded nucleic acid is a CRISPR-Cas protein, the first nucleic acid strand is crRNA, and the second strand is a tracrRNA.
Paragraph 74: The method of any one of paragraphs 1-3, wherein the first nucleic acid strand comprises the capture ligand, and the amplicon comprises the reporter molecule, and wherein the capture ligand is a single-stranded nucleic acid, and
   wherein said step of detecting the complex comprises:
      a. contacting the complex with a lateral flow device or micro-array plate, wherein the lateral flow device or the micro-array plate comprises a capture/test region comprising a nucleic acid strand immobilized thereon, wherein the immobilized nucleic acid strand comprises a nucleotide sequence substantially complementary to a nucleotide sequence of the single-stranded nucleic acid capture ligand; and
      b. detecting a detectable signal from the reporter molecule
Paragraph 75: The method of paragraph 74, wherein the capture ligand is attached to the 5′-end of the nucleic acid strand it is attached to.
Paragraph 76: The method of paragraph 74, wherein the capture ligand is attached to the 3′-end of the nucleic acid strand it is attached to.
Paragraph 77: The method of any one of paragraphs 1-3, wherein the first nucleic acid strand comprises the
   capture ligand, and the amplicon comprises the reporter molecule, and wherein the capture ligand is a toehold domain, and
   wherein said step of detecting the complex comprises:
      a. contacting the complex with a lateral flow device or micro-array plate, wherein the lateral flow device or the micro-array plate comprises a capture/test region comprising a nucleic acid strand immobilized thereon, wherein the immobilized nucleic acid strand comprises a nucleotide sequence substantially complementary to a region of the toehold domain; and
      b. detecting a detectable signal from the reporter molecule
Paragraph 78: The method of paragraph 77, wherein the toehold domain is attached to the 5′-end of the nucleic acid strand it is attached to.
Paragraph 79: The method of paragraph 78, wherein the toehold domain is attached to the 3′-end of the nucleic acid strand it is attached to.
Paragraph 80: The method of any one of paragraphs 1-3, wherein the method comprises:
   a. contacting amplicons from amplification of a plurality of target nucleic acids with a plurality of probes to form a plurality of complexes, where each complex comprises a probe and an amplicon,
      wherein the amplicon comprises a reporter molecule,
      wherein each probe comprises a first nucleic acid strand and a molecule complexed with the first nucleic acid strand, the molecule capable of localizing a single-strand nucleic acid strand to a double-stranded nucleic acid or enhancing the kinetics of hybridization between two single-stranded nucleic acids,
      wherein the first nucleic acid strand comprises a binding domain comprising a nucleotide sequence substantially complementary to at least a first portion of an amplicon,
      wherein the first nucleic acid strand comprises a capture ligand,
      wherein the amplicon and the capture ligand in a first member of the plurality of complexes is different from the amplicon and the capture ligand in a second member of the plurality of complexes, and
   b. detecting the complex comprising the probe and the amplicon, wherein said step of detecting the complex comprises:
      i. contacting the plurality of complexes with a lateral flow device or micro-array plate, wherein the lateral flow device or the micro-array plate comprises a plurality of capture/test regions, each capture region comprising a capture probe immobilized thereon and capable of binding with capture ligand, wherein the capture probe in at least two capture/test regions bind a different capture ligand from each other; and
      ii. detecting a detectable signal from the reporter molecules in the plurality of capture/test regions.
Paragraph 81: The method of any one of paragraphs 74-80, wherein the molecule capable of localizing a single-strand nucleic acid strand to a double-stranded nucleic acid or enhancing the kinetics of hybridization between two single-stranded nucleic acids is a CRISPR-Cas protein and the first nucleic acid strand is a guide RNA (sgRNA), optionally, the CRISPR-Cas protein lacks nuclease activity.
Paragraph 82: The method of any one of paragraphs 56-81, wherein the molecule capable of localizing a single-strand nucleic acid strand to a double-stranded nucleic acid or enhancing the kinetics of hybridization between two single-stranded nucleic acids is a CRISPR-Cas protein selected from the group consisting of C2c1, C2c3, Cas1, Cas100, Cas12a, Cas12b, Cas12c, Cas12d, Cas12e, Cas13a, Cas13b, Cas13c, Cas1B, Cas2, Cas3, Cas4, Cas5, Cas6, Cas7, Cas8, Cas9 (also known as Csnl and Csxl2), Casl, CaslB, CaslO, Cmr1, Cmr3, Cmr4, Cmr5, Cmr6, Cpf1, Csa5, Csa5, CsaX, Csb1, Csb2, Csb3, Csc1, Csc2, Cse1, Cse2, Csf1, Csf2, Csf3, Csf4, Csm2, Csm3, Csm4, Csm5, Csm6, Csn2, Csx1, Csx10, Csx14, Csx15, Csx16, Csx17, Csx3, Csy1, Csy2, Csy3, and homologues thereof, or modified versions thereof, optionally, the CRISPR-Cas protein lack nuclease activity.
Paragraph 83: The method of paragraph 82, wherein the CRISPR-Cas protein is Cas9, and wherein the Cas9 lacks nuclease activity.
Paragraph 84: The method of any one of paragraphs 4-83, wherein the reporter molecule is selected from the group consisting of fluorescent molecules, radioisotopes, chromophores, enzymes, enzyme substrates, chemiluminescent moieties, bioluminescent moieties, echogenic substances, non-metallic isotopes, optical reporters, paramagnetic metal ions, ferromagnetic metals, quantum dots (or semiconductor nanocrystals), nanoparticles (e.g. gold nanoparticles used in lateral flow assays, carbon nanoparticles), and latex and fluorescent beads.
Paragraph 85: The method of any one of paragraphs 4-84, wherein the reporter molecule is selected from the group consisting of 5-Carboxyfluorescein (5-FAM); 1,5 IAEDANS; 1,8-ANS; 4-Methylumbelliferone; 5-carboxy-2,7-dichlorofluorescein; 5-Carboxynapthofluorescein (pH 10); 5-Carboxytetramethylrhodamine (5-TAN/IRA); 5-FAM (5-Carboxyfluorescein); 5-Hydroxy Tryptamine (HAT); 5-ROX (carboxy-X-rhodamine); 5-TAMRA (5-Carboxytetramethylrhodamine); 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Aequorin (Photoprotein); Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; AMCA (Aminomethylcoumarin); AMCA-X; Aminoactinomycin D; Aminocoumarin; Anilin Blue; Anthrocyl stearate; APC-Cy7; APTS; Astrazon Brilliant Red 4G; Astrazon Orange R; Astrazon Red 6B; Astrazon Yellow 7 GFF; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Factamase; BFP blue shifted GFP (Y66H); BG-647; Bimane; Bisbenzamide; Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy FI; Bodipy FF ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Calcein; Calcein Blue; Calcium Crimson™; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CFDA; CFP—Cyan Fluorescent Protein; Chlorophyll; Chromomycin A; Chromomycin A; CMFDA; Coelenterazine; Coelenterazine cp; Coelenterazine f; Coelenterazine fcp; Coelenterazine h; Coelenterazine hep; Coelenterazine ip; Coelenterazine O; Coumarin Phalloidin; CPM Methylcoumarin; CTC; Cy2™; Cy3.1 8; Cy3.5™; Cy3™; Cy5.1 8; Cy5.5™; Cy5™; Cy7™; Cyan GFP; cyclic AMP Fluorosensor (FiCRhR); d2; Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di-16-ASP); DIDS; Dihydorhodamine 123 (DHR); DiO (DiOC18(3)); DiR; DiR (DiIC18(7)); Dopamine; DsRed; DTAF; DY-630-NHS; DY-635-NHS; EBFP; ECFP; EGFP; EFF 97; Eosin; Erythrosin; Erythrosin ITC; Ethidium homodimer-1 (EthD-1); Euchrysin; Europium (III) chloride; Europium; EYFP; Fast Blue; FDA; Feulgen (Pararosaniline); FITC; FL-645; Flazo Orange; Fluo-3; Fluo-4; Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura-2, high calcium; Fura-2, low calcium; Genacryl Brilliant Red B; Genacryl Brilliant Yellow 10GF; Genacryl Pink 3G; Genacryl Yellow 5GF; GFP (S65T); GFP red shifted (rsGFP); GFP wild type, non-UV excitation (wtGFP); GFP wild type, UV excitation (wtGFP); GFPuv; split proteins/enzymes (e.g., split GFP system such as GFP11/GFP10, sfCherry11/sfCherry₂11/sfCherry₃11, and lacZalpha); Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indodicarbocyanine (DiD); Indotricarbocyanine (DiR); Intrawhite Cf; JC-1; JO-JO-1; JO-PRO-1; FaserPro; Faurodan; EDS 751; Feucophor PAF; Feucophor SF; FeucophorWS; Fissamine Rhodamine; Fissamine Rhodamine B; FOFO-1; FO-PRO-1; Fucifer Yellow; Mag Green; Magdala Red (Phloxin B); Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxadidole; Noradrenaline; Nuclear Fast Red; Nuclear Yellow; Nylosan Brilliant Iavin EBG; Oregon Green™; Oregon Green 488-X; Oregon Green™ 488; Oregon Green™ 500; Oregon Green™ 514; Pacific Blue; Pararosaniline (Feulgen); PE-Cy5; PE-Cy7; PerCP; PerCP-Cy5.5; PE-TexasRed (Red 613); Phloxin B (Magdala Red); Phorwite AR; Phorwite BKF; Phorwite Rev; Phorwite RPA; Phosphine 3R; PhotoResist; Phycoerythrin B [PE]; Phycoerythrin R [PE]; PKH26; PKH67; PMIA; Pontochrome Blue Black; POPO-1; POPO-3; PO-PRO-1; PO-PRO-3; Primuline; Procion Yellow; Propidium Iodid (PI); PyMPO; Pyrene; Pyronine; Pyronine B; Pyrozal Brilliant Flavin 7GF; QSY 7; Quinacrine Mustard; Resorufm; RH 414; Rhod-2; Rhodamine; Rhodamine 110; Rhodamine 123; Rhodamine 5 GFD; Rhodamine 6G; Rhodamine B 540; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycoerythrin (PE); red shifted GFP (rsGFP, S65T); S65A; S65C; S65F; S65T; Sapphire GFP; Serotonin; Sevron Brilliant Red 2B; Sevron Brilliant Red 4G; Sevron Brilliant Red B; Sevron Orange; Sevron Yellow F; sgBFP™ (super glow BFP); sgGFP™ (super glow GFP); SITS; SITS (Primuline); SITS (Stilbene Isothiosulphonic Acid); SPQ (6-methoxy-N-(3-sulfopropyl)-quinolinium); Stilbene; Sulphorhodamine B can C; Sulphorhodamine G Extra; Tetracycline; Tetramethylrhodamine; Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC (TetramethylRodaminelsoThioCyanate); True Blue; TruRed; Ultralite; Uranine B; Uvitex SFC; wt GFP; WW 781; XF665; X-Rhodamine; XRITC; Xylene Orange; Y66F; Y66H; Y66W; Yellow GFP; YFP; YO-PRO-1; YO-PRO-3; YOYO-1; and YOYO-3.
Paragraph 86: The method of any one of paragraphs 4-85, wherein the reporter molecule is 5-FAM.
Paragraph 87: The method of any one of paragraphs 4-86, wherein the reporter molecule is attached to the 5′-end of the nucleic acid strand it is attached to.
Paragraph 88: The method of any one of paragraphs 4-86, wherein the reporter molecule is attached to the 3′-end of the nucleic acid strand it is attached to.
Paragraph 89: The method of any one of paragraphs 4-88, wherein the reporter molecule is a lateral flow detectable moiety.
Paragraph 90: The method of any one of paragraphs 4-89, wherein the nucleic acid strand comprising the reporter molecule comprises a second reporter molecule.
Paragraph 91: The method of paragraph 90, wherein one reporter molecule is at 5′-end and one reporter molecule is at 3′-end of the nucleic acid strand they are attached to.
Paragraph 92: The method of any one of paragraphs 4-91, wherein the capture ligand is selected from the group consisting of binding pairs, nucleic acids, nucleosides and nucleotides, vitamins, hormones, proteins, peptides, peptidomimetics, amino acids, monosaccharides, disaccharides, oligosaccharides, polysaccharides, lipopolysaccharides, polyols, receptors, and ligands for a receptor.
Paragraph 93: The method of any one of paragraphs 4-92, wherein the capture agent is selected from the group consisting of binding pairs and nucleic acids.
Paragraph 94: The method of any one of paragraphs 4-93, wherein the capture ligand is biotin, or digoxigenin (dig).
Paragraph 95: The method of any one of paragraphs 4-93, wherein the capture ligand is a nucleic acid (e.g. a single stranded nucleic acid).
Paragraph 96: The method of paragraph 95, wherein the capture ligand is a nucleic acid and comprises a toehold domain.
Paragraph 97: The method of any one of paragraphs 4-96, wherein the capture ligand is attached to the 5′-end of the nucleic acid strand it is attached to.
Paragraph 98: The method of any one of paragraphs 4-96, wherein the capture ligand is attached to the 3′-end of the nucleic acid strand it is attached to.
Paragraph 99: The method of any one of paragraphs 4-96, wherein the capture ligand is at an internal position of the nucleic acid strand it is attached to.
Paragraph 100: The method of any one of paragraphs 97-99, wherein the amplicon comprises at least one second capture ligand.
Paragraph 101: The method of paragraph 100, wherein one capture ligand is at the 5′-end and one capture ligand is the 3′-end of the nucleic acid strand they are attached to.
Paragraph 102: The method of paragraph 100, wherein one capture ligand is at the 5′-end and one capture ligand is at an internal position of the nucleic acid strand they are attached to.
Paragraph 103: The method of paragraph 100, wherein one capture ligand is at the 3′-end and one capture ligand is at an internal position of the nucleic acid strand they are attached to.
Paragraph 104: The method of paragraph 100, wherein one capture ligand is at the 5′-end, one capture ligand is at the 3′-end, and one capture ligand is at an internal position of the nucleic acid strand they are attached to.
Paragraph 105: The method of any one of paragraphs 1-104, further comprising a step of amplifying the target nucleic acid to produce the amplicon.
Paragraph 106: The method of paragraph 105, wherein said step of amplifying the target nucleic acid to produce the amplicon comprises isothermal amplification.
Paragraph 107: The method of paragraph 106, wherein the isothermal amplification is selected from the group consisting of: Recombinase Polymerase Amplification (RPA), Loop Mediated Isothermal Amplification (LAMP), Helicase-dependent isothermal DNA amplification (HDA), Rolling Circle Amplification (RCA), Nucleic acid sequence-based amplification (NASBA), strand displacement amplification (SDA), nicking enzyme amplification reaction (NEAR), Polymerase Spiral Reaction (PSR), Hybridization Chain Reaction (HCR), Primer Exchange Reaction (PER), Signal Amplification by Exchange Reaction (SABER), transcription-based amplification system (TAS), Self-sustained sequence replication reaction (3 SR), Single primer isothermal amplification (SPIA), and cross-priming amplification (CPA).
Paragraph 108: The method of any one of paragraphs 105-107, wherein the steps of step of amplifying the target nucleic acid to produce the amplicon and contacting the amplicon with the first probe are simultaneous.
Paragraph 109: The method of any one of paragraphs 105-107, wherein the steps of step of amplifying the target nucleic acid to produce the amplicon and contacting the amplicon with the first probe are sequential.
Paragraph 110: The method of any one of paragraphs 4-109, wherein each hybridization domain is independently from about 5 nucleotides to about 100 nucleotides in length.
Paragraph 111: The method of any one of paragraphs 4-110, wherein each hybridization domain is independently from about 10 nucleotides to about 75 nucleotides in length.
Paragraph 112: The method of any one of paragraphs 4-111, wherein each hybridization domain is independently from about 15 nucleotides to about 50 nucleotides in length.
Paragraph 113: The method of any one of paragraphs 1-112, wherein each binding domain is independently from about 5 nucleotides to about 100 nucleotides in length.
Paragraph 114: The method of any one of paragraphs 1-113, wherein each binding domain is independently from about 10 nucleotides to about 75 nucleotides in length.
Paragraph 115: The method of any one of paragraphs 1-114, wherein each binding domain is independently from about 15 nucleotides to about 50 nucleotides in length.
Paragraph 116: The method of any one of paragraphs 1-115, wherein the target nucleic acid is single-stranded.
Paragraph 117: The method of any one of paragraphs 1-115, wherein the target nucleic acid is double-stranded.
Paragraph 118: The method of any one of paragraphs 1-117, wherein the target nucleic acid is DNA.
Paragraph 119: The method of any of paragraphs 1-118, wherein the target nucleic acid is RNA.
Paragraph 120: The method of paragraph 119, wherein the method further comprises a step of preparing a cDNA from the target nucleic acid prior to preparing the amplicon.
Paragraph 121: The method of any one of paragraphs 1-120, wherein the amplicon is single-stranded.
Paragraph 122: The method of any one of paragraphs 1-121, wherein the amplicon is double-stranded.
Paragraph 123: A composition comprising a probe and a double-stranded or single-stranded amplicon from amplification of a target nucleic acid, wherein the first probe comprises a first nucleic acid strand and a molecule bound with the first nucleic acid strand and capable of localizing a single-strand nucleic acid strand to a double-stranded nucleic acid or enhancing the kinetics of hybridization between two single-stranded nucleic acids, and wherein the first nucleic acid strand comprises a binding domain comprising a nucleotide sequence substantially complementary to at least a first portion of the amplicon.
Paragraph 124: The composition of paragraph 123, wherein the first nucleic acid strand comprises a first hybridization domain linked with the binding domain and the first probe comprises a second nucleic acid strand hybridized with the first hybridization domain of the first nucleic acid strand.
Paragraph 125: The composition of paragraph 123, wherein the first nucleic acid strand comprises a first hybridization domain linked with the binding domain linked with a second hybridization domain, and the first probe further comprises a second nucleic acid strand hybridized with the first nucleic acid strand,
   wherein the second nucleic acid strand comprises a first hybridization domain linked with a non-hybridizing domain linked with a second hybridization domain,
   wherein the first hybridization domain of the first nucleic acid strand and the second hybridization domain of the second nucleic acid strand are hybridized with each other forming a first double-stranded region,
   wherein the second hybridization domain of the first nucleic acid strand and the first hybridization domain of the second nucleic acid strand are hybridized with each other forming a second double-stranded region,
   wherein the binding domain of the first nucleic acid strand and the non-hybridizing domain of the second nucleic acid strand do not hybridize to each other.
Paragraph 126: The composition of paragraph 124 or 125, wherein the first hybridization domain is linked to the 5′-end of the binding domain.
Paragraph 127: The composition of paragraph 124 or 125, wherein the first hybridization domain is linked to the 3′-end of the binding domain.
Paragraph 128: The composition of paragraph 123, wherein the first nucleic acid strand comprises two or more hybridization domains.
Paragraph 129: The composition of paragraph 126, wherein the two or more hybridization domains are separated by a non-hybridizing domain.
Paragraph 130: The composition of paragraph 123, wherein the first nucleic acid strand comprises two or more binding domains.
Paragraph 131: The composition of paragraph 130, wherein two or more binding domains are separated by a non-hybridizing domain.
Paragraph 132: The composition of any one of paragraphs 123-131, wherein the first nucleic acid strand comprises the reporter molecule and the amplicon comprises the capture ligand.
Paragraph 133: The composition of any one of paragraphs 123-131, wherein the amplicon comprises the reporter molecule and the first nucleic acid strand comprises the capture ligand.
Paragraph 134: The composition of paragraph 133, wherein the amplicon further comprises a second reporter molecule.
Paragraph 135: A composition comprising a probe and an amplicon from amplification of a target nucleic acid, wherein the probe comprises a first nucleic acid strand, a second nucleic acid strand, and a molecule capable of localizing a single-strand nucleic acid strand to a double-stranded nucleic acid, wherein the first nucleic acid strand comprises a binding domain comprising a nucleotide sequence substantially complementary to at least a first portion of the amplicon, wherein the first nucleic acid strand comprises a first hybridization domain linked with the binding domain and the second nucleic acid strand is hybridized with the first hybridization domain of the first nucleic acid strand, and wherein the first nucleic acid strand comprises a reporter molecule capable of producing a detectable signal and the second nucleic acid strand comprises a capture ligand.
Paragraph 136: The composition of paragraph 135, wherein the first nucleic acid strand and the second nucleic acid strand hybridized with each other to form a double-stranded structure comprising a single-stranded loop region,
   wherein the first nucleic acid strand comprises a first hybridization domain linked to the binding domain linked to a second hybridization domain,
   wherein the second nucleic acid strand comprises a first hybridization domain linked to a non-hybridizing domain linked to a second hybridization domain,
   wherein the first hybridization domain of the first nucleic acid strand and the second hybridization domain of the second nucleic acid strand are hybridized with each other forming a first double-stranded region,
   wherein the second hybridization domain of the first nucleic acid strand and the first hybridization domain of the second nucleic acid strand are hybridized with each other forming a second double-stranded region,
   wherein the binding domain of the first nucleic acid strand and the non-hybridizing domain of the second nucleic acid strand do not hybridize to each other.
Paragraph 137: A composition comprising a first probe, a second probe, and an amplicon from amplification of a target nucleic acid, wherein the first probe comprises a first nucleic acid strand and a first molecule capable of localizing a single-strand nucleic acid strand to a double-stranded nucleic acid, and wherein the first nucleic acid strand of the first probe comprises a binding domain comprising a nucleotide sequence substantially complementary to at least a first portion of the amplicon.
   wherein the second probe comprises a first nucleic acid strand and a first molecule capable of localizing a single-strand nucleic acid strand to a double-stranded nucleic acid, and wherein the first nucleic acid strand of the second probe comprises a binding domain comprising a nucleotide sequence substantially complementary to at least a second portion of the amplicon, and
   wherein one of the first nucleic acid strand of the first probe and the first nucleic acid strand of the second probe comprises a reporter molecule capable of producing a detectable signal and the other of the first nucleic acid strand of the first probe and the first nucleic acid strand of the second probe comprises a capture ligand.
Paragraph 138: The composition of paragraph 137, wherein the first and second portion of the amplicon are separated from each other by at least 5 nucleotides or at least 5 nucleotide base-pairs.
Paragraph 139: The composition of any one of paragraphs 137-138, wherein the one of the first and second portion of the amplicon is on a first strand of the amplicon and the other of the first and second portion of the amplicon is on a second strand of the amplicon.
Paragraph 140: The composition of any one of paragraphs 137-138, wherein the first and second portion of the amplicons are on the same strand of the amplicon.
Paragraph 141: A composition comprising probe and an amplicon from amplification of a target nucleic acid, wherein the probe comprises a first nucleic acid strand, a second nucleic acid strand, and a first molecule capable of localizing a single-strand nucleic acid strand to a double-stranded nucleic acid, and wherein the first nucleic acid strand of the first probe comprises a binding domain comprising a nucleotide sequence substantially complementary to at least a first portion of the amplicon, wherein the first nucleic acid strand comprises a first hybridization domain linked with the binding domain and the second nucleic acid strand is hybridized with the first hybridization domain of the first nucleic acid strand,
   wherein one of the first and second nucleic strand comprises a reporter molecule capable of producing a detectable signal and the other of the first and second nucleic strand comprises a quencher molecule, and wherein the quencher molecule quenches the detectable signal from the reporter molecule when the first and second nucleic acid strands are hybridized with each other,
   wherein the first hybridization domain and the binding domain of the first nucleic acid strand together comprise a nucleotide sequence substantially complementary to at least a portion of the amplicon.
Paragraph 142: The composition of paragraph 141, wherein the first nucleic acid strand comprises a first hybridization domain linked with the binding domain linked with a second hybridization domain,
   wherein the second nucleic acid strand comprises a first hybridization domain linked with a non-hybridizing domain linked with a second hybridization domain,
   wherein the first hybridization domain of the first nucleic acid strand and the second hybridization domain of the second nucleic acid strand are hybridized with each other forming a first double-stranded region,
   wherein the second hybridization domain of the first nucleic acid strand and the first hybridization domain of the second nucleic acid strand are hybridized with each other forming a second double-stranded region,
   wherein the binding domain of the first nucleic acid strand and the non-hybridizing domain of the second nucleic acid strand do not hybridize to each other.
Paragraph 143: The composition of any one of paragraphs 141-142, wherein the reporter molecule and the quencher molecule are a FRET pair.
Paragraph 144: The composition of any one of paragraphs 141-143, wherein the first nucleic acid strand comprises the reporter molecule.
Paragraph 145: The composition of any one of paragraphs 141-143, wherein the second nucleic acid strand comprises the reporter molecule.
Paragraph 146: The composition of any one of paragraphs 141-145, wherein the first hybridization domain is linked to the 5′-end of the binding domain.
Paragraph 147: The composition of any one of paragraphs 141-145, wherein the first hybridization domain is linked to the 3′-end of the binding domain.
Paragraph 148: The composition of any one of paragraphs 123-147, wherein the molecule capable of localizing a single-strand nucleic acid strand to a double-stranded nucleic acid or enhancing the kinetics of hybridization between two single-stranded nucleic acids is a CRISPR-Cas protein and the first nucleic acid strand is a guide RNA (sgRNA), optionally, the CRISPR-Cas protein lacks nuclease activity.
Paragraph 149: The composition of any one of paragraphs 123-147, wherein the molecule capable of localizing a single-stranded nucleic acid strand to a double-stranded nucleic acid enhancing the kinetics of hybridization between two single-stranded nucleic acids is a recombinase.
Paragraph 150: The composition of paragraph 149, wherein the recombinase is selected from the group consisting of RecA, UvsX, RadA, Rad51, Dmcl, or UvsY, Cre, Flp, Dre, SCre, VCre, Vika, B2, B3, KD, ΦC31, Bxb1, λ, HK022, HP1, γδ, ParA, Tn3, Gin, R4, TP901-1, TG1, PhiRv1, PhiBT1, SprA, XisF, TnpX, R, A118, spoIVCA, PhiMR11, SCCmec, TndX, XerC, XerD, XisA, Hin, Cin, mrpA, beta, PhiFC1, Fre, Clp, sTre, FimE, HbiFm, and homologues thereof, and modified versions thereof.
Paragraph 151: The composition of paragraph 150, wherein the recombinase is RecA.
Paragraph 152: The composition of any one of paragraphs 123-148, wherein the molecule capable of localizing a single-strand nucleic acid strand to a double-stranded nucleic acid is a CRISPR-Cas protein selected from the group consisting of C2c1, C2c3, Casl, Cas100, Cas12a, Cas12b, Cas12c, Cas12d, Cas12e, Cas13a, Cas13b, Cas13c, Cas1B, Cas2, Cas3, Cas4, Cas5, Cas6, Cas7, Cas8, Cas9 (also known as Csnl and Csxl2), Casl, CaslB, CaslO, Cmr1, Cmr3, Cmr4, Cmr5, Cmr6, Cpf1, Csa5, Csa5, CsaX, Csb1, Csb2, Csb3, Csc1, Csc2, Cse1, Cse2, Csf1, Csf2, Csf3, Csf4, Csm2, Csm3, Csm4, Csm5, Csm6, Csn2, Csx1, Csx10, Csx14, Csx15, Csx16, Csx17, Csx3, Csy1, Csy2, Csy3, and homologues thereof, or modified versions thereof, optionally, the CRISPR-Cas protein lack nuclease activity.
Paragraph 153: The composition of paragraph 152, wherein the CRISPR-Cas protein is Cas9, and wherein the Cas9 lacks nuclease activity.
Paragraph 154: The composition of paragraph 123, wherein the first nucleic acid strand comprises a first hybridization domain linked to the binding domain, and the first probe further comprises a second nucleic acid strand hybridized with the hybridization domain of the first strand,
   wherein the first or second nucleic strand comprises the capture ligand,
   wherein the amplicon comprises the reporter molecule capable of producing a detectable signal, and wherein the molecule capable of localizing a single-strand nucleic acid strand to a double-stranded nucleic acid is a CRISPR-Cas protein, the first nucleic acid strand is crRNA, and the second strand is a tracrRNA, optionally, the CRISPR-Cas protein lacks nuclease activity.
Paragraph 155: The composition of paragraph 123, wherein the first nucleic acid strand comprises the capture ligand, and the amplicon comprises the reporter molecule,
   wherein the capture ligand is a toehold domain, and
   wherein the molecule capable of localizing a single-strand nucleic acid strand to a double-stranded nucleic acid is a CRISPR-Cas protein and the first nucleic acid strand is a guide RNA (sgRNA), optionally, the CRISPR-Cas protein lacks nuclease activity.
Paragraph 156: The composition of any one of paragraphs 123-155, wherein the reporter molecule is selected from the group consisting of fluorescent molecules, radioisotopes, chromophores, enzymes, enzyme substrates, chemiluminescent moieties, bioluminescent moieties, echogenic substances, non-metallic isotopes, optical reporters, paramagnetic metal ions, ferromagnetic metals, quantum dots (or semiconductor nanocrystals), nanoparticles (e.g. gold nanoparticles used in lateral flow assays, carbon nanoparticles), and latex and fluorescent beads.
Paragraph 157: The composition of paragraph 156, wherein the reporter molecule is selected from the group consisting of 5-Carboxyfluorescein (5-FAM); 1,5 IAEDANS; 1,8-ANS; 4-Methylumbelliferone; 5-carboxy-2,7-dichlorofluorescein; 5-Carboxynapthofluorescein (pH 10); 5-Carboxytetramethylrhodamine (5-TAN/IRA); 5-FAM (5-Carboxyfluorescein); 5-Hydroxy Tryptamine (HAT); 5-ROX (carboxy-X-rhodamine); 5-TAMRA (5-Carboxytetramethylrhodamine); 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Aequorin (Photoprotein); Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; AMCA (Aminomethylcoumarin); AMCA-X; Aminoactinomycin D; Aminocoumarin; Anilin Blue; Anthrocyl stearate; APC-Cy7; APTS; Astrazon Brilliant Red 4G; Astrazon Orange R; Astrazon Red 6B; Astrazon Yellow 7 GFF; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Factamase; BFP blue shifted GFP (Y66H); BG-647; Bimane; Bisbenzamide; Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy FI; Bodipy FF ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Calcein; Calcein Blue; Calcium Crimson™; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CFDA; CFP—Cyan Fluorescent Protein; Chlorophyll; Chromomycin A; Chromomycin A; CMFDA; Coelenterazine; Coelenterazine cp; Coelenterazine f; Coelenterazine fcp; Coelenterazine h; Coelenterazine hep; Coelenterazine ip; Coelenterazine O; Coumarin Phalloidin; CPM Methylcoumarin; CTC; Cy2™; Cy3.1 8; Cy3.5™; Cy3™; Cy5.1 8; Cy5.5™; Cy5™; Cy7™; Cyan GFP; cyclic AMP Fluorosensor (FiCRhR); d2; Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di-16-ASP); DIDS; Dihydorhodamine 123 (DHR); DiO (DiOC18(3)); DiR; DiR (DiIC18(7)); Dopamine; DsRed; DTAF; DY-630-NHS; DY-635-NHS; EBFP; ECFP; EGFP; EFF 97; Eosin; Erythrosin; Erythrosin ITC; Ethidium homodimer-1 (EthD-1); Euchrysin; Europium (III) chloride; Europium; EYFP; Fast Blue; FDA; Feulgen (Pararosaniline); FITC; FL-645; Flazo Orange; Fluo-3; Fluo-4; Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura-2, high calcium; Fura-2, low calcium; Genacryl Brilliant Red B; Genacryl Brilliant Yellow 10GF; Genacryl Pink 3G; Genacryl Yellow 5GF; GFP (S65T); GFP red shifted (rsGFP); GFP wild type, non-UV excitation (wtGFP); GFP wild type, UV excitation (wtGFP); GFPuv; split proteins/enzymes (e.g., split GFP system such as GFP11/GFP10, sfCherry11/sfCherry₂11/sfCherry₃11, and lacZalpha); Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indodicarbocyanine (DiD); Indotricarbocyanine (DiR); Intrawhite Cf; JC-1; JO-JO-1; JO-PRO-1; FaserPro; Faurodan; EDS 751; Feucophor PAF; Feucophor SF; FeucophorWS; Fissamine Rhodamine; Fissamine Rhodamine B; FOFO-1; FO-PRO-1; Fucifer Yellow; Mag Green; Magdala Red (Phloxin B); Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxadidole; Noradrenaline; Nuclear Fast Red; Nuclear Yellow; Nylosan Brilliant Iavin EBG; Oregon Green™; Oregon Green 488-X; Oregon Green™ 488; Oregon Green™ 500; Oregon Green™ 514; Pacific Blue; Pararosaniline (Feulgen); PE-Cy5; PE-Cy7; PerCP; PerCP-Cy5.5; PE-TexasRed (Red 613); Phloxin B (Magdala Red); Phorwite AR; Phorwite BKF; Phorwite Rev; Phorwite RPA; Phosphine 3R; PhotoResist; Phycoerythrin B [PE]; Phycoerythrin R [PE]; PKH26; PKH67; PMIA; Pontochrome Blue Black; POPO-1; POPO-3; PO-PRO-1; PO-PRO-3; Primuline; Procion Yellow; Propidium Iodid (PI); PyMPO; Pyrene; Pyronine; Pyronine B; Pyrozal Brilliant Flavin 7GF; QSY 7; Quinacrine Mustard; Resorufm; RH 414; Rhod-2; Rhodamine; Rhodamine 110; Rhodamine 123; Rhodamine 5 GFD; Rhodamine 6G; Rhodamine B 540; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycoerythrin (PE); red shifted GFP (rsGFP, S65T); S65A; S65C; S65F; S65T; Sapphire GFP; Serotonin; Sevron Brilliant Red 2B; Sevron Brilliant Red 4G; Sevron Brilliant Red B; Sevron Orange; Sevron Yellow F; sgBFP™ (super glow BFP); sgGFP™ (super glow GFP); SITS; SITS (Primuline); SITS (Stilbene Isothiosulphonic Acid); SPQ (6-methoxy-N-(3-sulfopropyl)-quinolinium); Stilbene; Sulphorhodamine B can C; Sulphorhodamine G Extra; Tetracycline; Tetramethylrhodamine; Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC (TetramethylRodaminelsoThioCyanate); True Blue; TruRed; Ultralite; Uranine B; Uvitex SFC; wt GFP; WW 781; XF665; X-Rhodamine; XRITC; Xylene Orange; Y66F; Y66H; Y66W; Yellow GFP; YFP; YO-PRO-1; YO-PRO-3; YOYO-1; and YOYO-3.
Paragraph 158: The composition of paragraph 157, wherein the reporter molecule is 5-FAM.
Paragraph 159: The composition of any one of paragraphs 123-158, wherein the reporter molecule is attached to 5′-end of the nucleic acid strand it is attached to.
Paragraph 160: The composition of any one of paragraphs 123-158, wherein the reporter molecule is attached to 3′-end of the nucleic acid strand it is attached to.
Paragraph 161: The composition of any one of paragraphs 123-160, wherein the reporter molecule is a lateral flow detectable moiety.
Paragraph 162: The composition of any one of paragraphs 123-161, wherein the nucleic acid strand comprising the reporter molecule further comprises a second reporter molecule.
Paragraph 163: The composition of paragraph 162, wherein one reporter molecule is attached to the 5′-end and one reporter molecule is attached to the 3′-end of the nucleic acid strand they are attached to.
Paragraph 164: The composition of any one of paragraphs 123-163, wherein the capture ligand is selected from the group consisting of binding pairs, nucleic acids, nucleosides and nucleotides, vitamins, hormones, proteins, peptides, peptidomimetics, amino acids, monosaccharides, disaccharides, oligosaccharides, polysaccharides, lipopolysaccharides, polyols, receptors, ligand for a receptor,
Paragraph 165: The composition of any one of paragraphs 123-164, wherein the capture agent is selected from the group consisting of binding pairs and nucleic acids.
Paragraph 166: The composition of any one of paragraphs 123-165, wherein the capture ligand is biotin or digoxigenin.
Paragraph 167: The composition of any one of paragraphs 123-165, wherein the capture ligand is a nucleic acid (e.g. a single stranded nucleic acid).
Paragraph 168: The composition of paragraph 167, wherein the capture ligand is a nucleic acid and comprises a toehold domain.
Paragraph 169: The composition of any one of paragraphs 123-168, wherein the capture ligand is attached to the 5′-end of the nucleic acid strand it is attached to.
Paragraph 170: The composition of any one of paragraphs 123-168, wherein the capture ligand is attached to the 3′-end of the nucleic acid strand it is attached to.
Paragraph 171: The composition of any one of paragraphs 123-168, wherein the capture ligand is at an internal position of the nucleic acid strand it is attached to.
Paragraph 172: The method of any one of paragraphs 123-171, wherein the amplicon comprises at least one second capture ligand.
Paragraph 173: The method of paragraph 172, wherein one capture ligand is at the 5′-end and one capture ligand is the 3′-end of the nucleic acid strand they are attached to.
Paragraph 174: The method of paragraph 172, wherein one capture ligand is at the 5′-end and one capture ligand is at an internal position of the nucleic acid strand they are attached to.
Paragraph 175: The method of paragraph 172, wherein one capture ligand is at the 3′-end and one capture ligand is at an internal position of the nucleic acid strand they are attached to.
Paragraph 176: The method of paragraph 172, wherein one capture ligand is at the 5′-end, one capture ligand is at the 3′-end, and one capture ligand is at an internal position of the nucleic acid strand they are attached to.
Paragraph 177: The composition of any one of paragraphs 123-176, wherein each hybridization domain is independently from about 5 nucleotides to about 100 nucleotides in length.
Paragraph 178: The composition of any one of paragraphs 123-177, wherein each hybridization domain is independently from about 10 nucleotides to about 75 nucleotides in length.
Paragraph 179: The composition of any one of paragraphs 123-178, wherein each hybridization domain is independently from about 15 nucleotides to about 50 nucleotides in length.
Paragraph 180: The composition of any one of paragraphs 123-179, wherein each binding domain is independently from about 5 nucleotides to about 100 nucleotides in length.
Paragraph 181: The composition of any one of paragraphs 123-180, wherein each binding domain is independently from about 10 nucleotides to about 75 nucleotides in length.
Paragraph 182: The composition of any one of paragraphs 123-181, wherein each binding domain is independently from about 15 nucleotides to about 50 nucleotides in length.
Paragraph 183: The composition of any one of paragraphs 123-182, wherein the composition further comprising reagents for preparing an amplicon from the target nucleic acid.
Paragraph 184: The composition of any one of paragraphs 123-183, wherein the composition further comprises a DNA polymerase.
Paragraph 185: The composition of any one of paragraphs 123-184, wherein the composition further comprises reverse transcriptase.
Paragraph 186: The composition of any one of paragraphs 123-185, wherein the composition further comprises dNTPs.
Paragraph 187: The composition of any one of paragraphs 123-186, wherein the composition further comprises a buffer.
Paragraph 188: The composition of any one of paragraphs 123-187, further composition comprises means for detecting a detectable signal from the reporter molecule.
Paragraph 189: The composition of any one of paragraphs 123-188, wherein the composition is on a surface of a lateral flow device or micro-array plate.

### Kits

Paragraph 190: A kit comprising a primer set for preparing a double-stranded or single-stranded amplicon from a target nucleic acid, and a first probe,
   wherein the first probe comprises a first nucleic acid strand and a molecule bound with the first nucleic acid strand, wherein the molecule is capable of localizing a single-strand nucleic acid strand to a double-stranded nucleic acid or enhancing the kinetics of hybridization between two single-stranded nucleic acids,
   wherein the first nucleic acid strand comprises a binding domain comprising a nucleotide sequence substantially complementary to at least a first portion of the amplicon prepared from a target nucleic acid using the primer set,
   wherein the first nucleic acid strand comprises a reporter molecule capable of producing a detectable signal and at least one primer in the primer set comprises a capture ligand, or the first nucleic acid strand comprises capture ligand and at least one primer in the primer set comprises a reporter molecule capable of producing a detectable signal.
Paragraph 191: The kit of paragraph 190, wherein the first nucleic acid strand comprises a first hybridization domain linked with the binding domain and the first probe comprises a second nucleic acid strand hybridized with the first hybridization domain of the first nucleic acid strand.
Paragraph 192: The kit of paragraph 190, wherein the first nucleic acid strand comprises a first hybridization domain linked with the binding domain linked with a second hybridization domain, and the first probe further comprises a second nucleic acid strand hybridized with the first nucleic acid strand,
   wherein the second nucleic acid strand comprises a first hybridization domain linked with a non-hybridizing domain linked with a second hybridization domain,
   wherein the first hybridization domain of the first nucleic acid strand and the second hybridization domain of the second nucleic acid strand are hybridized with each other forming a first double-stranded region,
   wherein the second hybridization domain of the first nucleic acid strand and the first hybridization domain of the second nucleic acid strand are hybridized with each other forming a second double-stranded region,
   wherein the binding domain of the first nucleic acid strand and the non-hybridizing domain of the second nucleic acid strand do not hybridize to each other.
Paragraph 193: The kit of paragraph 191 or 192, wherein the first hybridization domain is linked to the 5′-end of the binding domain.
Paragraph 194: The kit of paragraph 191 or 192, wherein the first hybridization domain is linked to the 3′-end of the binding domain.
Paragraph 195: The kit of any one of paragraphs 190-194, wherein the first nucleic acid strand comprises the reporter molecule and the primer in the primer set comprises the capture ligand.
Paragraph 196: The kit of any one of paragraphs 190-195, wherein the primer in the primer set comprises the reporter molecule and the first nucleic acid strand comprises the capture ligand.
Paragraph 197: The kit of paragraph 190, wherein the first probe further comprises a second nucleic acid strand, wherein the first nucleic acid strand comprises a first hybridization domain linked with the binding domain and the second nucleic acid strand is hybridized with the first hybridization domain of the first nucleic acid strand, and wherein the first nucleic acid strand comprises a reporter molecule capable of producing a detectable signal and the second nucleic acid strand comprises a capture ligand.
Paragraph 198: The kit of paragraph 197, wherein the first nucleic acid strand and the second nucleic acid strand hybridized with each other to form a double-stranded structure comprising a single-stranded loop region,
   wherein the first nucleic acid strand comprises a first hybridization domain linked to the binding domain linked to a second hybridization domain,
   wherein the second nucleic acid strand comprises a first hybridization domain linked to a non-hybridizing domain linked to a second hybridization domain,
   wherein the first hybridization domain of the first nucleic acid strand and the second hybridization domain of the second nucleic acid strand are hybridized with each other forming a first double-stranded region,
   wherein the second hybridization domain of the first nucleic acid strand and the first hybridization domain of the second nucleic acid strand are hybridized with each other forming a second double-stranded region,
   wherein the binding domain of the first nucleic acid strand and the non-hybridizing domain of the second nucleic acid strand do not hybridize to each other.
Paragraph 199: The kit of paragraph 190, further comprising a second probe, wherein the second probe comprises a first nucleic acid strand and a first molecule capable of localizing a single-strand nucleic acid strand to a double-stranded nucleic acid, and wherein the first nucleic acid strand of the second probe comprises a binding domain comprising a nucleotide sequence substantially complementary to at least a second portion of the amplicon prepared from a target nucleic acid using the primer set, and
   wherein one of the first nucleic acid strand of the first probe and the first nucleic acid strand of the second probe comprises a reporter molecule capable of producing a detectable signal and the other of the first nucleic acid strand of the first probe and the first nucleic acid strand of the second probe comprises a capture ligand.
Paragraph 200: The kit of paragraph 199, wherein the first and second portion of the amplicon are separated from each other by at least 5 nucleotides or at least 5 nucleotide base-pairs.
Paragraph 201: The kit of any one of paragraphs 199-200, wherein the one of the first and second portion of the amplicon is on a first strand of the amplicon and the other of the first and second portion of the amplicon is on a second strand of the amplicon.
Paragraph 202: The kit of any one of paragraphs 199-200, wherein the first and second portion of the amplicons are on same strand of the amplicon.
Paragraph 203: A kit comprising a primer set for preparing a double-stranded or single-stranded amplicon from a target nucleic acid, and a first probe,
   wherein the first probe comprises a first nucleic acid strand, a second nucleic acid strand, and a molecule capable of localizing a single-strand nucleic acid strand to a double-stranded nucleic acid or enhancing the kinetics of hybridization between two single-stranded nucleic acids,
   wherein the first nucleic acid strand comprises a first hybridization domain linked with a binding domain,
   wherein the second nucleic acid strand is hybridized with the first hybridization domain of the first nucleic acid strand,
   wherein one of the first and second nucleic strand comprises a reporter molecule capable of producing a detectable signal and the other of the first and second nucleic strand comprises a quencher molecule, and wherein the quencher molecule quenches the detectable signal from the reporter molecule when the first and second nucleic acid strands are hybridized with each other.
Paragraph 204: The kit of paragraph 203, wherein the first nucleic acid strand comprises a first hybridization domain linked with the binding domain linked with a second hybridization domain,
   wherein the second nucleic acid strand comprises a first hybridization domain linked with a non-hybridizing domain linked with a second hybridization domain,
   wherein the first hybridization domain of the first nucleic acid strand and the second hybridization domain of the second nucleic acid strand are hybridized with each other forming a first double-stranded region,
   wherein the second hybridization domain of the first nucleic acid strand and the first hybridization domain of the second nucleic acid strand are hybridized with each other forming a second double-stranded region,
   wherein the binding domain of the first nucleic acid strand and the non-hybridizing domain of the second nucleic acid strand do not hybridize to each other.
Paragraph 205: The kit of any one of paragraphs 203-204, wherein the reporter molecule and the quencher molecule are a FRET pair.
Paragraph 206: The kit of any one of paragraphs 203-205, wherein the first nucleic acid strand comprises the reporter molecule.
Paragraph 207: The kit of any one of paragraphs 203-206, wherein the second nucleic acid strand comprises the reporter molecule.
Paragraph 208: The kit of any one of paragraphs 203-207, wherein the first hybridization domain is linked to the 5′-end of the binding domain.
Paragraph 209: The kit of any one of paragraphs 203-208, wherein the first hybridization domain is linked to the 3′-end of the binding domain.
Paragraph 210: The kit of any one of paragraphs 190-209, wherein the molecule capable of localizing a single-strand nucleic acid strand to a double-stranded nucleic acid or enhancing the kinetics of hybridization between two single-stranded nucleic acids is a CRISPR-Cas protein and the first nucleic acid strand is a guide RNA (sgRNA), optionally, the CRISPR-Cas protein lacks nuclease activity.
Paragraph 211: The kit of any one of paragraphs 190-210, wherein the molecule capable of localizing a single-strand nucleic acid strand to a double-stranded nucleic acid enhancing the kinetics of hybridization between two single-stranded nucleic acids is a recombinase.
Paragraph 212: The kit of paragraph 211, wherein the recombinase is selected from the group consisting of RecA, UvsX, RadA, Rad51, Dmcl, UvsX, Cre, Flp, Dre, SCre, VCre, Vika, B2, B3, KD, ΦC31, Bxb1, λ, HK022, HP1, γδ, ParA, Tn3, Gin, R4, TP901-1, TG1, PhiRv1, PhiBT1, SprA, XisF, TnpX, R, A118, spoIVCA, PhiMR11, SCCmec, TndX, XerC, XerD, XisA, Hin, Cin, mrpA, beta, PhiFC1, Fre, Clp, sTre, FimE, HbiFm, and homologues thereof, and modified versions thereof.
Paragraph 213: The kit of paragraph 212, wherein the recombinase is RecA.
Paragraph 214: The kit of any one of paragraphs 190-210, wherein the molecule capable of localizing a single-strand nucleic acid strand to a double-stranded nucleic acid enhancing the kinetics of hybridization between two single-stranded nucleic acids is a CRISPR-Cas protein selected from the group consisting of C2c1, C2c3, Cas1, Cas100, Cas12a, Cas12b, Cas12c, Cas12d, Cas12e, Cas13a, Cas13b, Cas13c, Cas1B, Cas2, Cas3, Cas4, Cas5, Cas6, Cas7, Cas8, Cas9 (also known as Csn1 and Csx12), Casl, CaslB, CaslO, Cmr1, Cmr3, Cmr4, Cmr5, Cmr6, Cpf1, Csa5, Csa5, CsaX, Csb1, Csb2, Csb3, Csc1, Csc2, Cse1, Cse2, Csf1, Csf2, Csf3, Csf4, Csm2, Csm3, Csm4, Csm5, Csm6, Csn2, Csx1, Csx10, Csx14, Csx15, Csx16, Csx17, Csx3, Csy1, Csy2, Csy3, and homologues thereof, or modified versions thereof, optionally, the CRISPR-Cas protein lack nuclease activity.
Paragraph 215: The kit of paragraph 214, wherein the CRISPR-Cas protein is Cas9, and wherein the Cas9 lacks nuclease activity.
Paragraph 216: The kit of paragraph 190, wherein the first nucleic acid strand comprises a first hybridization domain linked to the binding domain, and the first probe further comprises a second nucleic acid strand hybridized with the hybridization domain of the first strand,
   wherein the first or second nucleic strand comprises the capture ligand,
   wherein a primer in the primer set comprises the reporter molecule capable of producing a detectable signal, and wherein the molecule capable of localizing a single-strand nucleic acid strand to a double-stranded nucleic acid or enhancing the kinetics of hybridization between two single-stranded nucleic acids is a CRISPR-Cas protein, the first nucleic acid strand is crRNA, and the second strand is a tracrRNA, optionally, the CRISPR-Cas protein lacks nuclease activity.
Paragraph 217: The kit of paragraph 190, wherein the first nucleic acid strand comprises the capture ligand, and a primer in the primer set comprises the reporter molecule,
   wherein the capture ligand is a toehold domain, and
   wherein the molecule capable of localizing a single-strand nucleic acid strand to a double-stranded nucleic acid or enhancing the kinetics of hybridization between two single-stranded nucleic acids is a CRISPR-Cas protein and the first nucleic acid strand is a guide RNA (sgRNA), optionally, the CRISPR-Cas protein lacks nuclease activity.
Paragraph 218: The kit of any one of paragraphs 190-217, wherein the reporter molecule is selected from the group consisting of fluorescent molecules, radioisotopes, chromophores, enzymes, enzyme substrates, chemiluminescent moieties, bioluminescent moieties, echogenic substances, non-metallic isotopes, optical reporters, paramagnetic metal ions, ferromagnetic metals, quantum dots (or semiconductor nanocrystals), nanoparticles (e.g. gold nanoparticles used in lateral flow assays, carbon nanoparticles), and latex and fluorescent beads.
Paragraph 219: The kit of any one of paragraphs 190-218, wherein the reporter molecule is selected from the group consisting of 5-Carboxyfluorescein (5-FAM); 1,5 IAEDANS; 1,8-ANS; 4-Methylumbelliferone; 5-carboxy-2,7-dichlorofluorescein; 5-Carboxynapthofluorescein (pH 10); 5-Carboxytetramethylrhodamine (5-TAN/IRA); 5-FAM (5-Carboxyfluorescein); 5-Hydroxy Tryptamine (HAT); 5-ROX (carboxy-X-rhodamine); 5-TAMRA (5-Carboxytetramethylrhodamine); 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Aequorin (Photoprotein); Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; AMCA (Aminomethylcoumarin); AMCA-X; Aminoactinomycin D; Aminocoumarin; Anilin Blue; Anthrocyl stearate; APC-Cy7; APTS; Astrazon Brilliant Red 4G; Astrazon Orange R; Astrazon Red 6B; Astrazon Yellow 7 GFF; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Factamase; BFP blue shifted GFP (Y66H); BG-647; Bimane; Bisbenzamide; Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy FI; Bodipy FF ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Calcein; Calcein Blue; Calcium Crimson™; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CFDA; CFP—Cyan Fluorescent Protein; Chlorophyll; Chromomycin A; Chromomycin A; CMFDA; Coelenterazine; Coelenterazine cp; Coelenterazine f; Coelenterazine fcp; Coelenterazine h; Coelenterazine hep; Coelenterazine ip; Coelenterazine O; Coumarin Phalloidin; CPM Methylcoumarin; CTC; Cy2™; Cy3.1 8; Cy3.5™; Cy3™; Cy5.1 8; Cy5.5™; Cy5™; Cy7™; Cyan GFP; cyclic AMP Fluorosensor (FiCRhR); d2; Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di-16-ASP); DIDS; Dihydorhodamine 123 (DHR); DiO (DiOC18(3)); DiR; DiR (DiIC18(7)); Dopamine; DsRed; DTAF; DY-630-NHS; DY-635-NHS; EBFP; ECFP; EGFP; EFF 97; Eosin; Erythrosin; Erythrosin ITC; Ethidium homodimer-1 (EthD-1); Euchrysin; Europium (III) chloride; Europium; EYFP; Fast Blue; FDA; Feulgen (Pararosaniline); FITC; FL-645; Flazo Orange; Fluo-3; Fluo-4; Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura-2, high calcium; Fura-2, low calcium; Genacryl Brilliant Red B; Genacryl Brilliant Yellow 10GF; Genacryl Pink 3G; Genacryl Yellow 5GF; GFP (S65T); GFP red shifted (rsGFP); GFP wild type, non-UV excitation (wtGFP); GFP wild type, UV excitation (wtGFP); GFPuv; split proteins/enzymes (e.g., split GFP system such as GFP11/GFP10, sfCherry11/sfCherry₂11/sfCherry₃11, and lacZalpha); Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indodicarbocyanine (DiD); Indotricarbocyanine (DiR); Intrawhite Cf; JC-1; JO-JO-1; JO-PRO-1; FaserPro; Faurodan; EDS 751; Feucophor PAF; Feucophor SF; FeucophorWS; Fissamine Rhodamine; Fissamine Rhodamine B; FOFO-1; FO-PRO-1; Fucifer Yellow; Mag Green; Magdala Red (Phloxin B); Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxadidole; Noradrenaline; Nuclear Fast Red; Nuclear Yellow; Nylosan Brilliant Iavin EBG; Oregon Green™; Oregon Green 488-X; Oregon Green™ 488; Oregon Green™ 500; Oregon Green™ 514; Pacific Blue; Pararosaniline (Feulgen); PE-Cy5; PE-Cy7; PerCP; PerCP-Cy5.5; PE-TexasRed (Red 613); Phloxin B (Magdala Red); Phorwite AR; Phorwite BKF; Phorwite Rev; Phorwite RPA; Phosphine 3R; PhotoResist; Phycoerythrin B [PE]; Phycoerythrin R [PE]; PKH26; PKH67; PMIA; Pontochrome Blue Black; POPO-1; POPO-3; PO-PRO-1; PO-PRO-3; Primuline; Procion Yellow; Propidium Iodid (PI); PyMPO; Pyrene; Pyronine; Pyronine B; Pyrozal Brilliant Flavin 7GF; QSY 7; Quinacrine Mustard; Resorufm; RH 414; Rhod-2; Rhodamine; Rhodamine 110; Rhodamine 123; Rhodamine 5 GFD; Rhodamine 6G; Rhodamine B 540; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycoerythrin (PE); red shifted GFP (rsGFP, S65T); S65A; S65C; S65F; S65T; Sapphire GFP; Serotonin; Sevron Brilliant Red 2B; Sevron Brilliant Red 4G; Sevron Brilliant Red B; Sevron Orange; Sevron Yellow F; sgBFP™ (super glow BFP); sgGFP™ (super glow GFP); SITS; SITS (Primuline); SITS (Stilbene Isothiosulphonic Acid); SPQ (6-methoxy-N-(3-sulfopropyl)-quinolinium); Stilbene; Sulphorhodamine B can C; Sulphorhodamine G Extra; Tetracycline; Tetramethylrhodamine; Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC (TetramethylRodaminelsoThioCyanate); True Blue; TruRed; Ultralite; Uranine B; Uvitex SFC; wt GFP; WW 781; XF665; X-Rhodamine; XRITC; Xylene Orange; Y66F; Y66H; Y66W; Yellow GFP; YFP; YO-PRO-1; YO-PRO-3; YOYO-1; and YOYO-3.
Paragraph 220: The kit of paragraph 219, wherein the reporter molecule is 5-FAM.
Paragraph 221: The kit of any one of paragraphs 190-220, wherein the reporter molecule is attached to the 5′-end of the nucleic acid strand it is attached to.
Paragraph 222: The kit of any one of paragraphs 190-220, wherein the reporter molecule is attached to the 3′-end of the nucleic acid strand it is attached to.
Paragraph 223: The kit of any one of paragraphs 190-222, wherein the reporter molecule is a lateral flow detectable moiety.
Paragraph 224: The kit of any one of paragraphs 190-223, wherein the nucleic acid strand comprising the reporter molecule further comprises a second reporter molecule.
Paragraph 225: The kit of paragraph 224, wherein one reporter molecule is attached to the 5′-end and one reporter molecule is attached to the 3′-end of the nucleic acid strand they are attached to.
Paragraph 226: The kit of any one of paragraphs 190-225, wherein the capture ligand is selected from the group consisting of binding pairs, nucleic acids, nucleosides and nucleotides, vitamins, hormones, proteins, peptides, peptidomimetics, amino acids, monosaccharides, disaccharides, oligosaccharides, polysaccharides, lipopolysaccharides, polyols, receptors, ligand for a receptor,
Paragraph 227: The kit of any one of paragraphs 190-226, wherein the capture agent is selected from the group consisting of binding pairs and nucleic acids.
Paragraph 228: The kit of any one of paragraphs 190-227, wherein the capture ligand is biotin or digoxigenin.
Paragraph 229: The kit of any one of paragraphs 190-227, wherein the capture ligand is a nucleic acid (e.g. a single stranded nucleic acid).
Paragraph 230: The kit of paragraph 229, wherein the capture ligand is a nucleic acid and comprises a toehold domain.
Paragraph 231: The kit of any one of paragraphs 190-230, wherein the capture ligand is attached to the 5′-end of the nucleic acid strand it is attached to.
Paragraph 232: The kit of any one of paragraphs 190-230, wherein the capture ligand is attached to the 3′-end of the nucleic acid strand it is attached to.
Paragraph 233: The kit of any one of paragraphs 190-230, wherein the capture ligand is at an internal position of the nucleic acid strand it is attached to.
Paragraph 234: The method of any one of paragraphs 190-233, wherein the amplicon comprises at least one second capture ligand.
Paragraph 235: The method of paragraph 234, wherein one capture ligand is at the 5′-end and one capture ligand is the 3′-end of the nucleic acid strand they are attached to.
Paragraph 236: The method of paragraph 234, wherein one capture ligand is at the 5′-end and one capture ligand is at an internal position of the nucleic acid strand they are attached to.
Paragraph 237: The method of paragraph 234, wherein one capture ligand is at the 3′-end and one capture ligand is at an internal position of the nucleic acid strand they are attached to.
Paragraph 238: The method of paragraph 234, wherein one capture ligand is at the 5′-end, one capture ligand is at the 3′-end, and one capture ligand is at an internal position of the nucleic acid strand they are attached to.
Paragraph 239: The kit of any one of paragraphs 190-238, wherein each hybridization domain is independently from about 5 nucleotides to about 100 nucleotides in length.
Paragraph 240: The kit of any one of paragraphs 190-239, wherein each hybridization domain is independently from about 10 nucleotides to about 75 nucleotides in length.
Paragraph 241: The kit of any one of paragraphs 190-240, wherein each hybridization domain is independently from about 15 nucleotides to about 50 nucleotides in length.
Paragraph 242: The kit of any one of paragraphs 190-241, wherein each binding domain is independently from about 5 nucleotides to about 100 nucleotides in length.
Paragraph 243: The kit of any one of paragraphs 190-242, wherein each binding domain is independently from about 10 nucleotides to about 75 nucleotides in length.
Paragraph 244: The kit of any one of paragraphs 190-243, wherein each binding domain is independently from about 15 nucleotides to about 50 nucleotides in length.
Paragraph 245: The kit of any one of paragraphs 190-244, wherein the kit further comprising reagents for preparing an amplicon from the target nucleic acid.
Paragraph 246: The kit of any one of paragraphs 190-245, wherein the kit further comprises a DNA polymerase.
Paragraph 247: The kit of any one of paragraphs 190-246, wherein the kit further comprises reverse transcriptase.
Paragraph 248: The kit of any one of paragraphs 190-247, wherein the kit further comprises dNTPs.
Paragraph 249: The kit of any one of paragraphs 190-248, wherein the kit further comprises a buffer.
Paragraph 250: The kit of any one of paragraphs 190-249, further kit comprises means for detecting a detectable signal from the reporter molecule.
Paragraph 251: The kit of any one of paragraphs 190-250, wherein the kit further comprises a lateral flow device or micro-array plate.
Paragraph 252: The method, composition or kit of any one of the preceding paragraphs, wherein the reporter molecule is linked to the nucleic acid strand it is attached to by a linker.
Paragraph 253: The method, composition or kit of any one of the preceding paragraphs, wherein the capture ligand is linked to the nucleic acid strand it is attached to by a linker.
Paragraph 254: The method, composition or kit of any one of the preceding paragraphs, wherein the quencher molecule is selected from the group consisting of Deep Dark Quenchers (Eurogentec), DABCYL, TAMRA BHQ quenchers (e.g., BHQ-0, BHQ-1, BHQ-2, BHQ-3, and BHQ-10), BBQ®-650, ECLIPSE, Iowa Black® quenchers, QSY (e.g., QSY 21, QSY 15, QSY 7, and QSY 9), and IRDye® QC-1.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### Definitions

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADAM Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

The terms “increased”, “increase”, or “enhance” are all used herein to mean an increase by a statically significant amount. In some embodiments, the terms “increased”, “increase”, or “enhance” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “hybridizing”, “hybridize”, “hybridization”, “annealing”, or “anneal” are used interchangeably in reference to the pairing of complementary nucleic acids using any process by which a strand of nucleic acid joins with a complementary strand through base pairing to form a hybridization complex. In other words, the term “hybridization” refers to the process in which two single-stranded polynucleotides bind non-covalently to form a stable double-stranded polynucleotide. Furthermore, the term “hybridize” refers to the phenomenon of a single-stranded nucleic acid or region thereof forming hydrogen-bonded base pair interactions with either another single stranded nucleic acid or region thereof (intermolecular hybridization) or with another single-stranded region of the same nucleic acid (intramolecular hybridization). Hybridization is governed by the base sequences involved, with complementary nucleobases forming hydrogen bonds, and the stability of any hybrid being determined by the identity of the base pairs (e.g., G:C base pairs being stronger than A:T base pairs) and the number of contiguous base pairs, with longer stretches of complementary bases forming more stable hybrids. The term “hybridization” may also refer to triple-stranded hybridization. The resulting (usually) double-stranded polynucleotide is a “hybrid” or “duplex.”

In some embodiments of the various aspects described herein, the step of contacting the probe with the target nucleic acid can comprise heating and/or cooling. For example, a reaction comprising the target nucleic acid and the probe can be heated and then cooled to promote hybridization.

It is noted that the hybridization step can be carried out in the same reaction vessel used for preparing the amplified product. Alternatively, the amplified product can be isolated or purified from the amplification reaction prior to the hybridization step. In other words, the amplification step and the hybridization steps are in different reaction vessels.

“Hybridization conditions” will typically include salt concentrations of less than about 1 M, more usually less than about 500 mM and even more usually less than about 200 mM. Hybridization temperatures can be as low as 5° C., but are typically greater than 22° C., more typically greater than about 30° C., and often in excess of about 37° C. Hybridizations are usually performed under stringent conditions, i.e., conditions under which a probe will hybridize to its target subsequence. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the Tm for the specific sequence at a defined ionic strength and pH. Exemplary stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM Na phosphate, 5 mM EDTA, pH 7.4) and a temperature of 25-30° C. are suitable for allele-specific probe hybridizations. For stringent conditions, see for example, Sambrook, Fritsche and Maniatis, Molecular Cloning A Laboratory Manual, 2nd Ed. Cold Spring Harbor Press (1989) and Anderson Nucleic Acid Hybridization, 1st Ed., BIOS Scientific Publishers Limited (1999). “Hybridizing specifically to” or “specifically hybridizing to” or like expressions refer to the binding, duplexing, or hybridizing of a molecule substantially to or only to a particular nucleotide sequence or sequences under stringent conditions when that sequence is present in a complex mixture.

The term “substantially identical” means two or more nucleotide sequences have at least 65%, 70%, 80%, 85%, 90%, 95%, or 97% identical nucleotides. In some embodiments, “substantially identical” means two or more nucleotide sequences have the same identical nucleotides.

The term “substantial complementary” or “substantially complementary” as used herein refers both to complete complementarity of binding nucleic acids, in some cases referred to as an identical sequence, as well as complementarity sufficient to achieve the desired binding of nucleic acids. Correspondingly, the term “complementary hybrids” encompasses substantially complementary hybrids.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions may include: 400 mM NaCl, 40 mM PIPES pH 6.4, 1 mM EDTA, 50° C. or 70° C. for 12-16 hours followed by washing. Other conditions, such as physiologically relevant conditions as may be encountered inside an organism, can apply. The skilled person will be able to determine the set of conditions most appropriate for a test of complementarity of two sequences in accordance with the ultimate application of the hybridized nucleotides.

As used herein, the term “complementary,” in the context of an oligonucleotide (i.e., a sequence of nucleotides such as an oligonucleotide primer or a target nucleic acid) refers to standard Watson/Crick base pairing rules. For example, the sequence “5′-A-G-T-C-3′” is complementary to the sequence “3′-T-C-A-G-S′.” Certain nucleotides not commonly found in natural nucleic acids or chemically synthesized may be included in the nucleic acids described herein; these include but not limited to base and sugar modified nucleosides, nucleotides, and nucleic acids, such as inosine, isocytosine and isoguanine. “Complementary” sequences, as used herein, may also include, or be formed entirely from, non-Watson-Crick base pairs and/or base pairs formed from non-natural and modified nucleotides, in as far as the above requirements with respect to their ability to hybridize are fulfilled. Such non-Watson-Crick base pairs include, but not limited to, G:U Wobble or Hoogsteen base pairing. In other words, complementarity need not be perfect; stable duplexes may contain mismatched base pairs, degenerative, or unmatched nucleotides. Those skilled in the art of nucleic acid technology can determine duplex stability empirically considering a number of variables including, for example, the length of the oligonucleotide, base composition and sequence of the oligonucleotide, incidence of mismatched base pairs, ionic strength, other hybridization buffer components and conditions.

Complementarity may be partial in which only some of the nucleotide bases of two nucleic acid strands are matched according to the base pairing rules. Complementarity may be complete or total where all of the nucleotide bases of two nucleic acid strands are matched according to the base pairing rules. Complementarity may be absent where none of the nucleotide bases of two nucleic acid strands are matched according to the base pairing rules. In some embodiments of any of the aspects, two nucleic acid strands are at least 50%, at least 55%, at least 60%, at least 65%, at least 70%, at least 75%, at least 80%, at least 85%, at least 90%, at least 95%, at least 96%, at least 97%, at least 98%, at least 99%, or 100% complementary. The degree of complementarity between nucleic acid strands has significant effects on the efficiency and strength of hybridization between nucleic acid strands. This is of particular importance in detection methods that depend upon binding between nucleic acids.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third non-target entity. A reagent specific for a given target is one that exhibits specific binding for that target under the conditions of the assay being utilized.

The term “statistically significant” or “significantly” refers to statistical significance and generally means a two standard deviations (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%. In some embodiments of any of the aspects, the term “about” when used in connection with percentages can mean±5% (e.g., ±4%, ±3%, ±2%, ±1%) of the value being referred to.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Where a range of values is provided, each numerical value between the upper and lower limits of the range is contemplated and disclosed herein.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

The technology described herein is further illustrated by the following examples which in no way should be construed as being further limiting. It will be apparent to those skilled in the relevant art that various modifications, additions, substitutions, and the like can be performed without altering the spirit or scope of the invention, and such modifications and variations are encompassed within the scope of the invention as defined in the claims which follow.

### EXAMPLES

### Example 1

The ability to rapidly detect nucleic acids with high sensitivity and specificity on a portable platform has the potential to revolutionize diagnosis and monitoring for many diseases, provide epidemiological information, sense biomarkers, and serve as a generalizable scientific tool. Although many methods have been developed for detecting nucleic acids, they suffer from trade-offs in terms of sensitivity, specificity, simplicity, and speed. For example, real-time quantitative PCR (qPCR) is currently the gold standard for many nucleic acid-based diagnostics, but these assays, however, require expensive thermal cycling equipment and are typically run in centralized laboratories. Other approaches, such as new methods coupling isothermal nucleic acid amplification with portable platforms, offer high detection specificity in a point-of-care (POC) setting, but sample-to-result time is still often on the order of hours. As nucleic acid detection becomes increasingly relevant for a variety of healthcare applications, improved methods and devices for the specific and rapid detection of DNA/RNA sequences are needed for accurate detection.

The technology described in this example encompasses compositions related to RecA-based sensors and methods for sensitive, specific, and reliable detection of target nucleic acids of interest. The recombinases of the RecA family are DNA-pairing proteins—they bind to one DNA segment, align it with homologous sequences in another DNA segment, promote an exchange of DNA strands and then dissociate. When RecA-based DNA recognition is coupled with isothermal amplification reactions and immunochromatographic lateral flow assays, low-cost, simplified detection of nucleic acids is achieved. These assays are designed to specifically target SARS-CoV-2, the causative agent of the COVID-19 pandemic, and a variety of biomarkers associated with radiation exposure. The RecA-based detection mechanism can be used in several different schemes, utilizing the unique strand displacement of RecA in a variety of ways. Furthermore, these assays can be used for visible detection of SARS-CoV-2 down to as few as 10 copies of RNA in under 15 minutes, all at a fixed temperature.

Provided herein are methods of detecting target nucleic acids. The invention provides a method of detecting the presence of pathogen-associated nucleic acids in a sample. The invention is particularly well suited for developing products for detection of pathogenic RNA or DNA, specifically at the point-of-care and in low-resource settings. Specifically, the invention allows for the detection of pathogenic nucleic acids in 15 minutes, which rivals antigen diagnostic tests (which often suffer lack of sensitivity and specificity). Advantages of the methods provided are multifold. For example, results can be obtained in under 20 minutes, and the assays require limited infrastructure (i.e. pipettes, hot plates). Consequently, the diagnostic tests do not need to take place at centralized labs and can provide results either at home or in the span of a doctor's visit for patients who are in urgent need of care. The assays are nucleic acid based, resulting in improved specificity over antibody tests and may be quickly redesigned for other pathogens. Additionally, the liquid-based reactions in which amplification and detection are combined in a single test tube enable rapid detection without any user steps. Test results are visible by eye using simple, low cost lateral flow strips.

In 2006, Piepenburg et al. developed the recombinase polymerase amplification (RPA) technology using proteins involved in cellular DNA synthesis, recombination and repair, which is currently commercialized by TwistDx (www.twistdx.co.uk). While both this current disclosure and Piepenburg et al exploits the properties of the bacterial RecA protein and related properties to invade double-stranded DNA (dsDNA) with single stranded homologous DNA (ssDNA), the applications of both technologies are fundamentally different. Piepenburg et al. use the ssDNA to permit sequence specific priming of DNA polymerase reactions, whereas in the various aspects described herein, the RecA protein is used to create more complex interactions between longer ssDNA fragments, thus allowing for nucleic acid detection and subsequent readout. To date, RecA has not been used in any dsDNA detection schemes.

The primary amino group of DNA has also been used extensively to attach a variety of modifiers such as fluorescent dyes, biotin, digoxigenin, and other molecules, for both fluorescent and immunochromatographic recognition of DNA. Often, however, the modifications are made directly to the DNA through incorporation via primers during amplification. RecA-filament formation provided in this disclosure utilizes the strand displacement properties of RecA to allow for signal detection, whether through immunochromatographic or fluorescent means. We also demonstrate use of the Taq RecA protein, a much more thermal stable protein than the E. coli recA, which enables signals to be produced directly in LAMP reactions at temperatures of at 65° C.

Piepenburg O, Williams C H, Stemple D L, Armes N A. DNA detection using recombination proteins. PLoS Biol. 2006 July; 4(7):e204. doi: 10.1371/journal.pbio.0040204. PMID: 16756388; PMCID: PMC1475771.

The RecA-family of recombinases promote the exchange of genetic information between two homologous DNA molecules. Their functional form in recombination reactions is a right-handed helical filament bound to DNA. This filament is usually formed on single-stranded (ss)DNA as the first reaction step. The bound ssDNA is then aligned with a homologous double-stranded (ds)DNA, and a strand-exchange reaction ensues in which the complementary strand of the DNA duplex is transferred to the originally bound ssDNA. RecA recombinases are also DNA-dependent ATPases, and ATP is hydrolysed during strand-exchange reactions. RecA protein polymerizes cooperatively and nonspecifically on DNA to form a helical filament that is the active species in DNA-strand exchange reactions. Filaments are formed by rapid polymerization of RecA monomers 5′ to 3′ (relative to the ssDNA to which it is bound) on single stranded DNA or duplex DNA possessing a single-stranded gap.

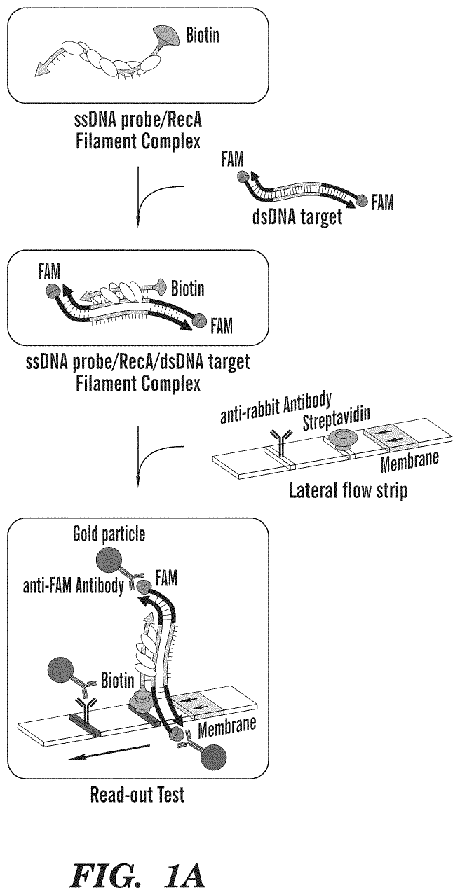

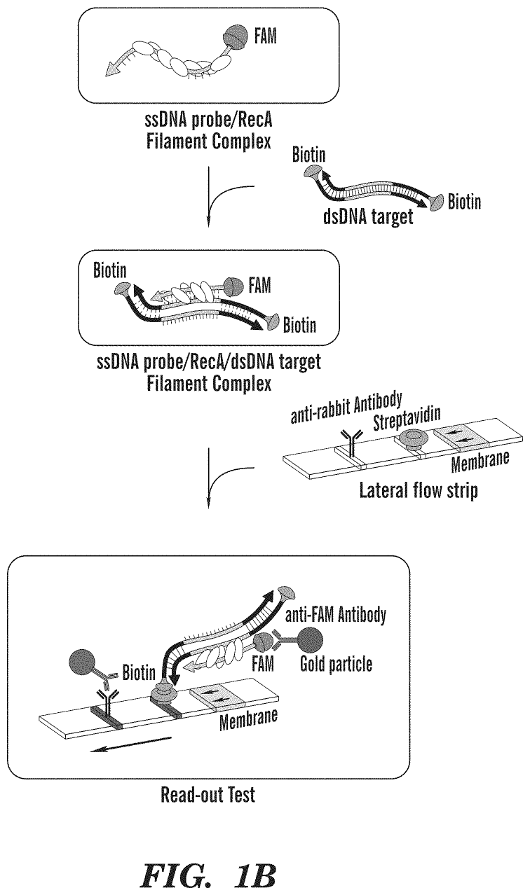

A recombinase platform for nucleic acid detection using RecA is shown in FIGS. 1A and 1B. A ssDNA probe, modified with a biotin (FIG. 1A) or FAM (FIG. 1B) moiety, is incubated with RecA to create a filament complex. The ssDNA probe comprises one or more single-stranded homology domains that are complementary to a dsDNA target or the reverse complement. A dsDNA target modified with an additional FAM or biotin moiety (FIG. 1A, 1B, respectively), is subjected to the ssDNA probe/RecA filament complex, which results in the formation of a triple-stranded filament. Detection of the triple stranded RecA filament is conducted on a lateral flow strip with a streptavidin sample line and colloidal gold particles coated with an anti-FAM antibody. When the immunochromatographic assay is ran, binding of the gold nanoparticle allows for visual detection of either the modified dsDNA target or ssDNA probe.

In this system, the dsDNA target can be generated by isothermal amplification. Nucleic acids obtained from a biological sample of a subject (saliva, nasopharyngeal swab, blood, serum, or other matrix), are subjected to isothermal amplification by reactions that include nucleic acid sequence-based amplification (NASBA), recombinase polymerase amplification (RPA), or loop mediated amplification (LAMP). Primers of isothermal assays incorporate a detectable moiety (such as biotin, FAM) to allow for subsequent detection of pathogen-specific dsDNA amplicon. Use of a ssDNA probe when creating the triple stranded RecA filament also reduces the potential for false positive results by ensuring that the correct sequence is generated from isothermal amplification, a common failure mode for assays that employ isothermal amplification alone.

### 1. Recombinase-Based Diagnostic Assay for SARS-CoV-2 Detection

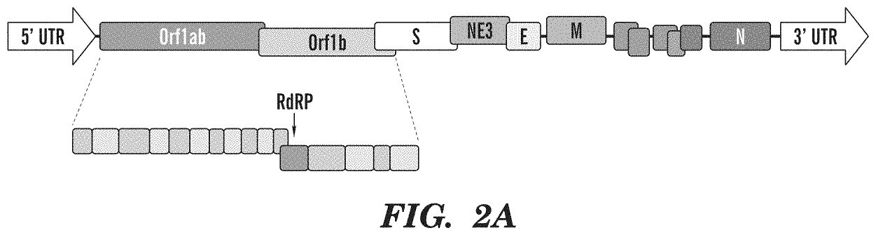

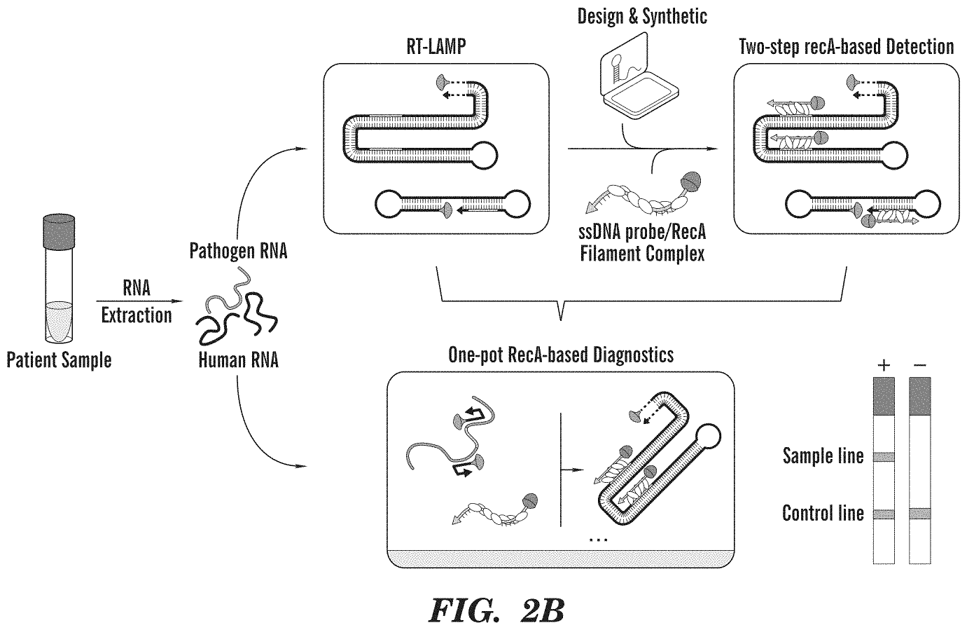

In the first aspect, a diagnostic platform is demonstrated for detecting SARS-CoV-2-specific nucleic acids (FIGS. 2A and 2B). The primary target regions for the assays include Orf1b, RdRP, spike gene (S), and nucleocapsid gene (N) (FIG. 2A); an additional assay was developed to include a human control target, actin B (ACTB). LAMP assays were developed, and primers were designed with both FAM and biotin modifications. ssDNA probes were designed to bind either the forward or reverse orientation of target sequences. Two different assays were developed based on the overall scheme: in one case, RNA is extracted from patient samples, then isothermal amplification and ssDNA probe incubation are performed separately. In another case, isothermal amplification and ssDNA probe incubation are performed simultaneously. The two different iterations of RecA-based detection are termed ‘one-pot’ and ‘two-pot’ reactions, respectively (FIG. 2B).

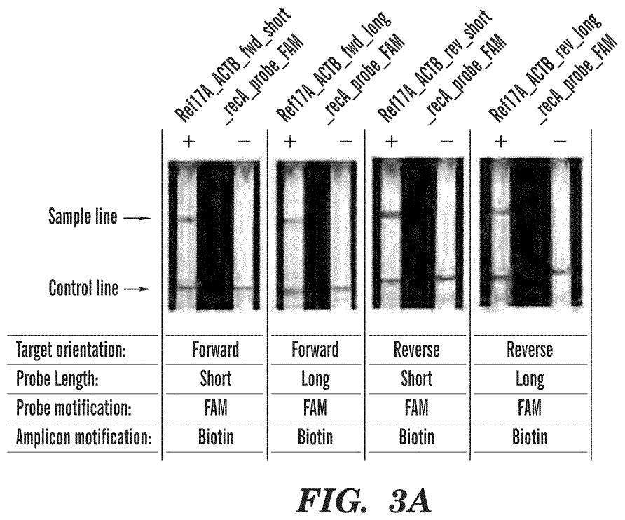

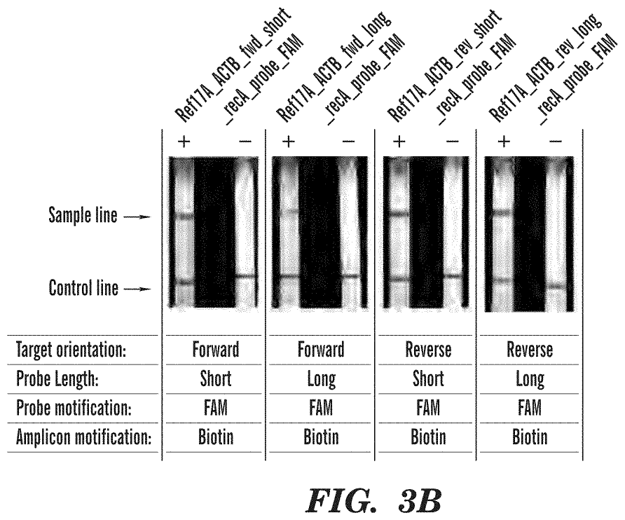

A human control target, ACTB, was screened to determine optimal target orientation, probe length, probe modification, and amplicon modification. Computationally designed probes, targeting ACTB sequences with little secondary structure, were screened for function by challenging them with synthetic versions of ACTB genomic targets after in vitro transcription. To demonstrate the potential of various RecA proteins applied to the system, E coli RecA was first used in a two-pot reaction. Reverse-transcriptase LAMP (RT-LAMP) was performed to generate amplicon modified with biotin, and then FAM-modified probes were used for visual detection on a lateral flow strip (LFS) readout with the E coli RecA (FIG. 3A). Using a variety of target orientations and probe lengths (sort: 51-54 nucleotides; long: 64 nucleotides), signal was acquired in all positive reactions. To test a one-pot reaction scheme, Thermus aquaticus RecA protein (Taq RecA), a thermostable isoform of the RecA protein, was used to test simultaneous RT-LAMP and ssDNA probe/RecA incubation (FIG. 3B). In conjunction with the same probes as in FIG. 3A, signal bands were visible for all combinations.

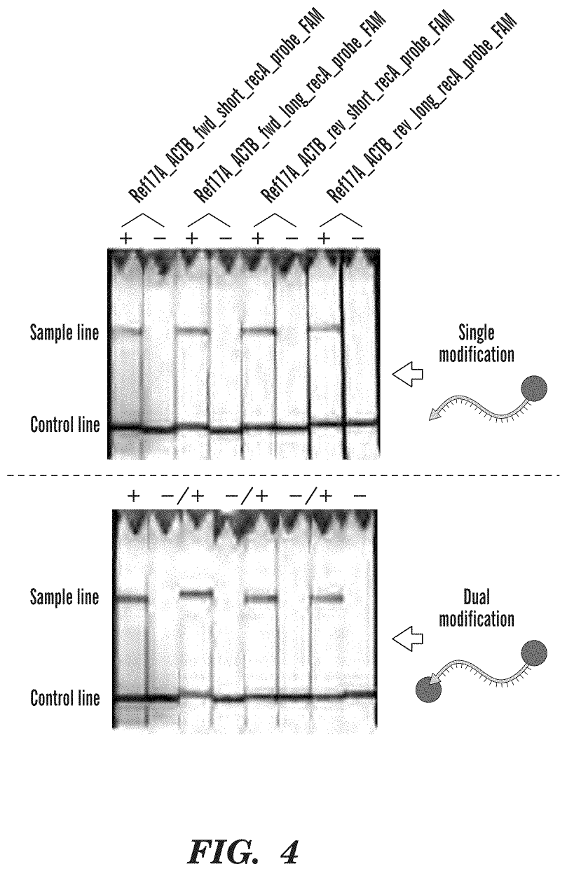

To examine whether signal intensity is dependent on the number of modifications of the probes, the ssDNA oligos were modified with FAM on either the 3′ or both 3′ and 5′ probe ends (FIG. 4). Although dual modification provides a stronger signal at the sample line, probes with a single modification give adequate signal to differentiate between a positive and negative result.

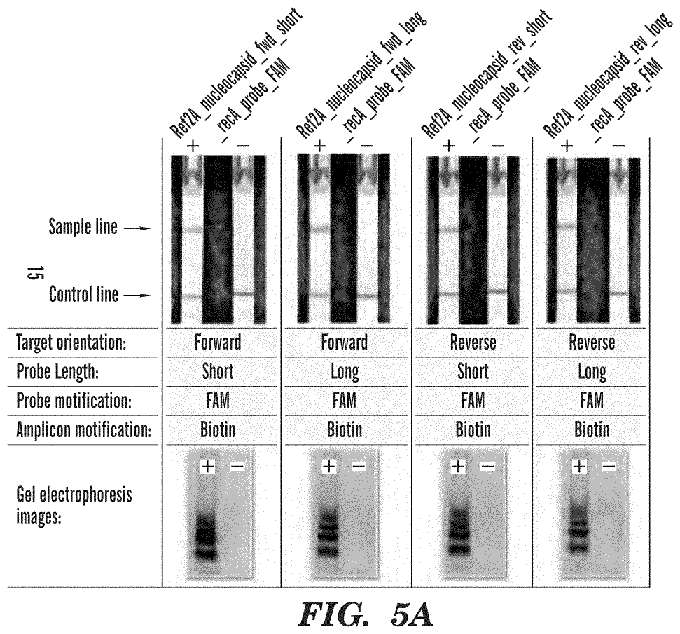

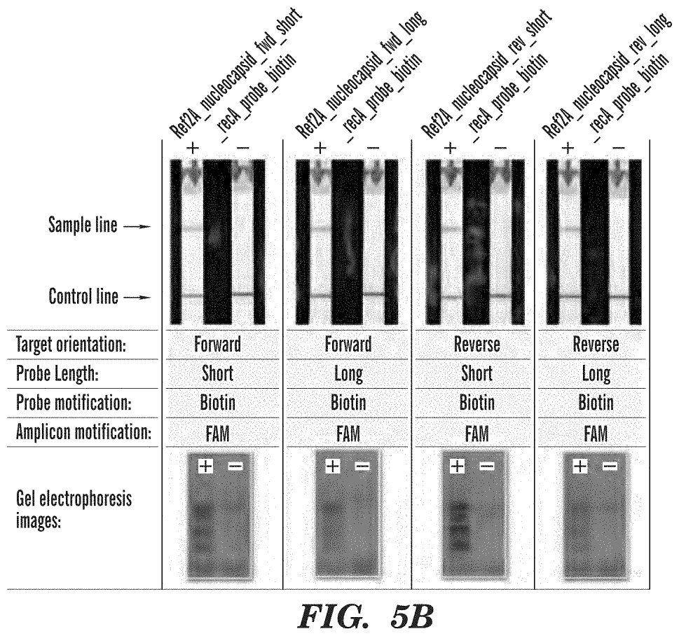

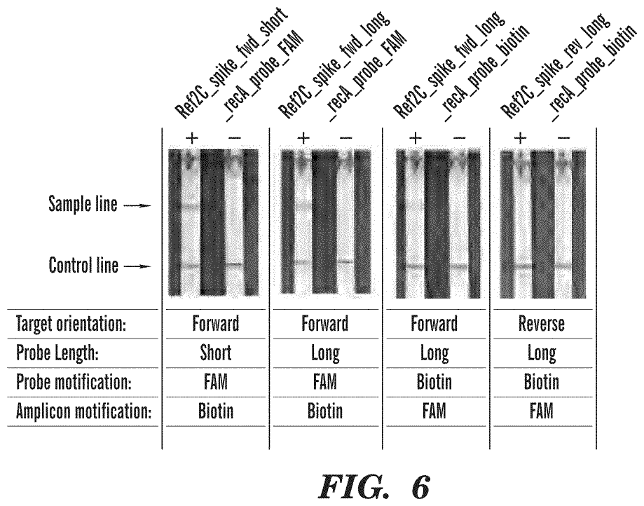

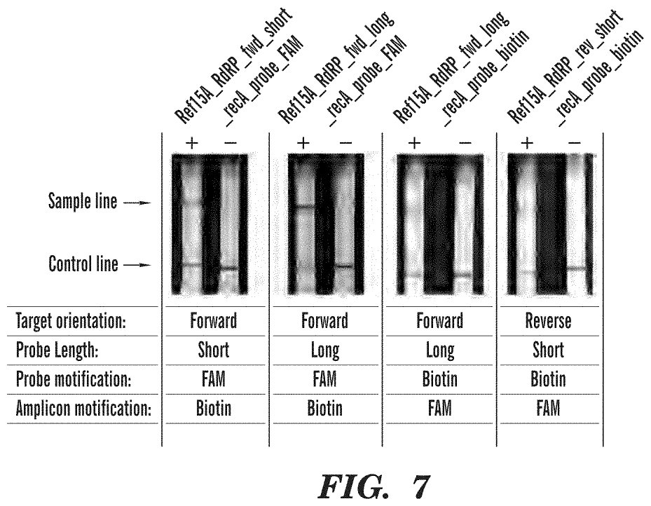

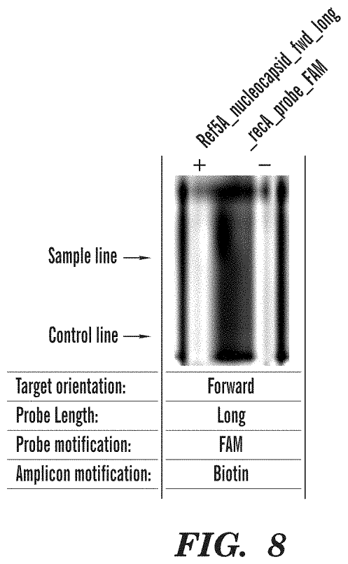

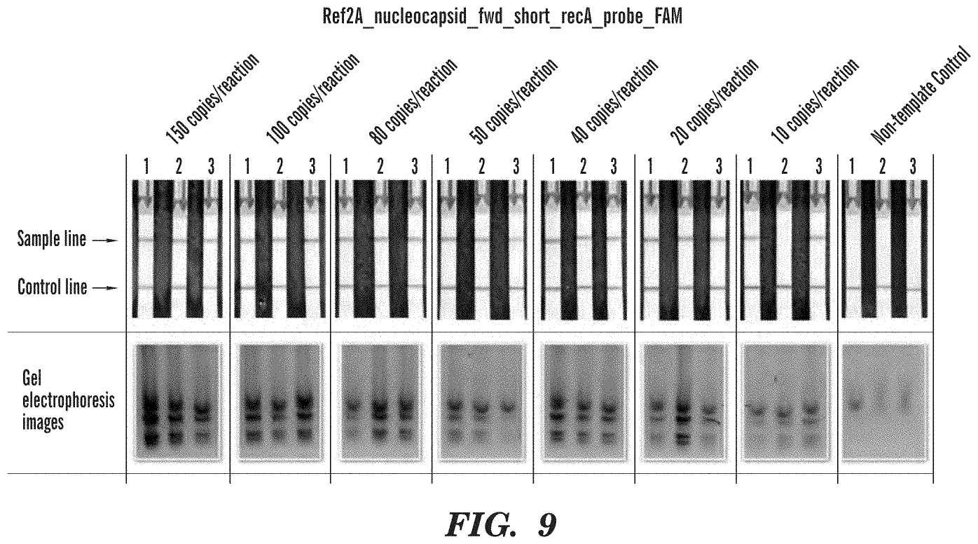

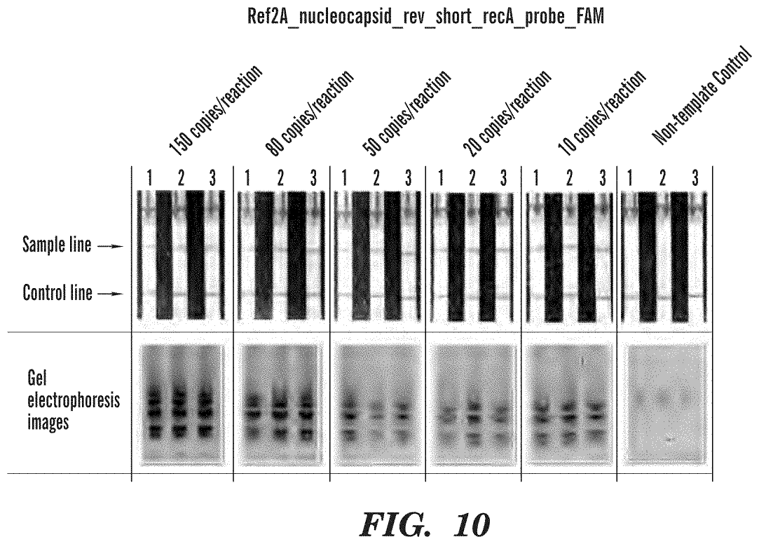

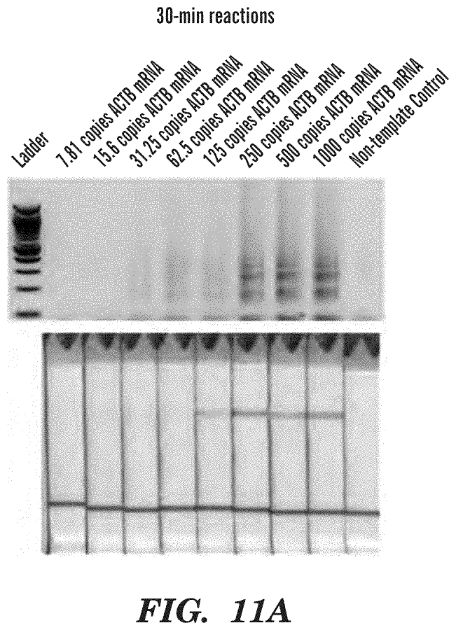

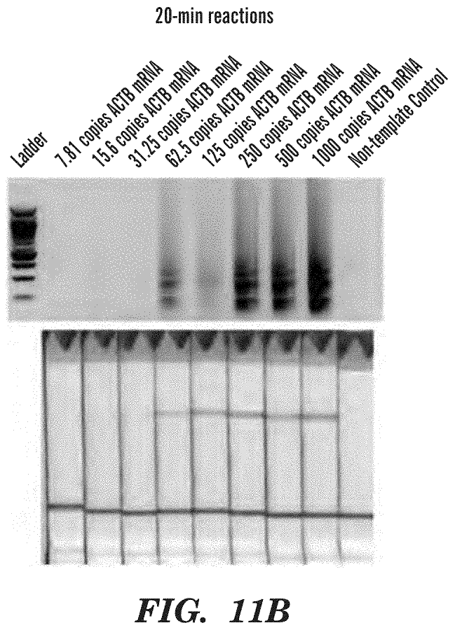

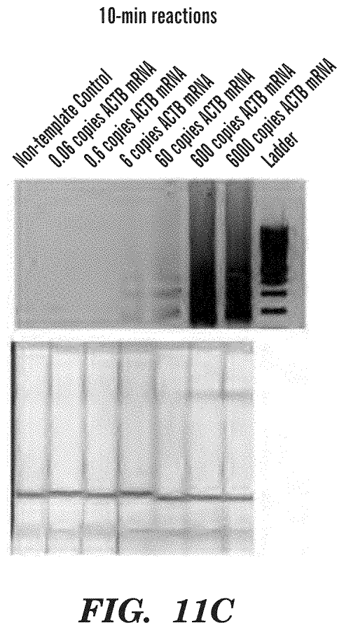

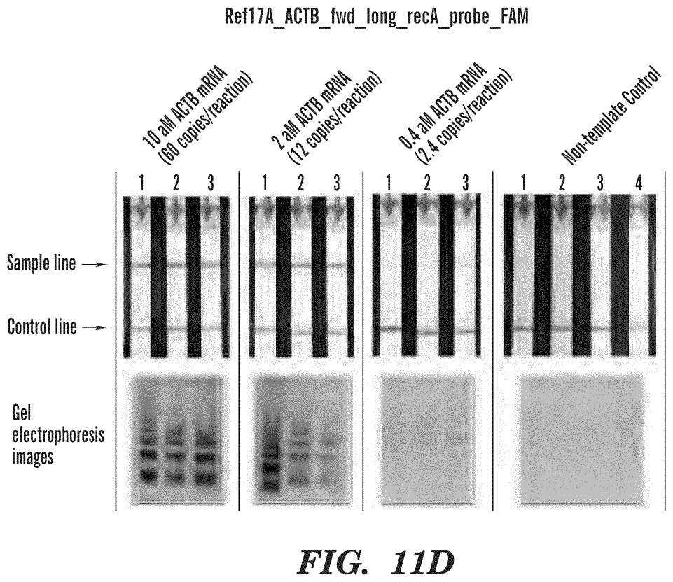

LAMP assays were then developed for a highly conserved region of the SARS-CoV-2 genome, the nucleocapsid protein (FIGS. 5 and 8), as well as the spike gene (FIG. 6), and the RdRP gene (FIG. 7). As examined for the ACTB control, each target was screened for varying target orientation (forward/reverse), probe length, and LAMP amplicon/probe modification. Across all targets, the lateral flow strip readout signal intensity was examined, and the probe that yielded the highest signal intensity was chosen to conduct limit of detection studies. For example, for nucleocapsid gene target Ref2A, the forward short and reverse short probes with the FAM modification provided a high signal intensity when applied against biotinylated LAMP product. When each of these probes were then taken to elucidate the lower limit of detection (LOD) the reverse short probe had demonstrated a LOD of 10 copies/reaction, while the forward short probe was sensitive on the lateral flow strips (LFS) only to 20 copies/reaction. However, the gel electrophoresis image demonstrated successful amplification for each assay at 10 copies/reaction, which indicates the sensitivity of the reaction is related to the probe size and orientation. When a different target was chosen, actin beta (ACTB) mRNA control target, sensitivity was demonstrated to be as low as 12 copies/reaction, with agreement between gel electrophoresis images for the RT-LAMP product and the LFS results. For all assays performed in the LOD study, the assays were run in two-pot reactions; from sample-to-result, the total time for assay completion was only 60 minutes (45 minutes RT-LAMP).

To demonstrate the robustness and sensitivity of the two-pot reaction (RT-LAMP coupled with RecA/probe incubation), RT-LAMP was conducted for 30 minutes, 20 minutes, and 10 minutes. Using the optimal probe sequence for the human control ACTB, the LOD was found to be 62.5 copies of ACTB/reaction at 30 minutes, and the LOD on paper for a 10-minute RT-LAMP reaction was found to be 6 copies of ACTB/reaction at 10 minutes.

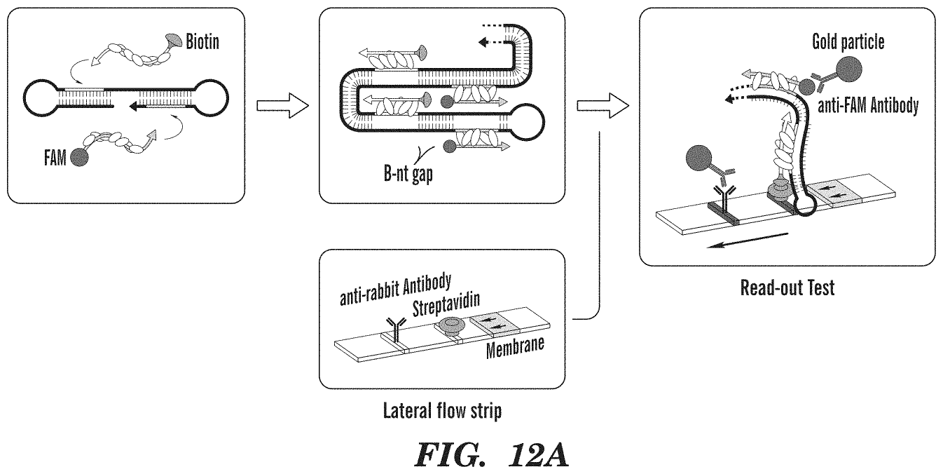

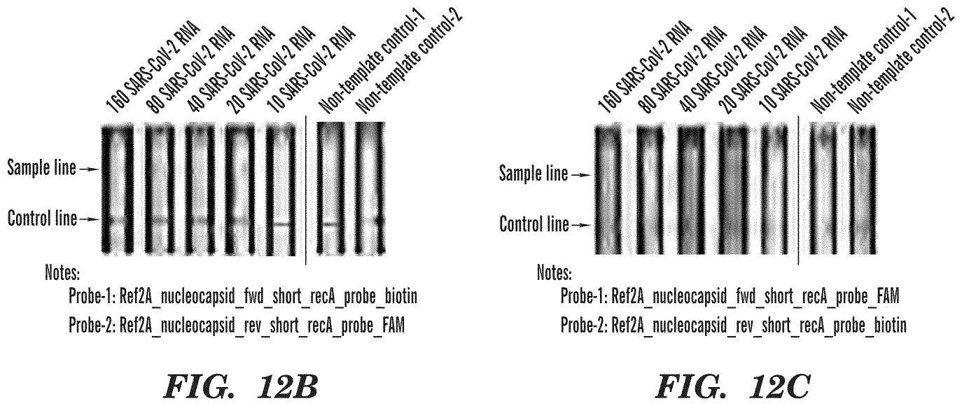

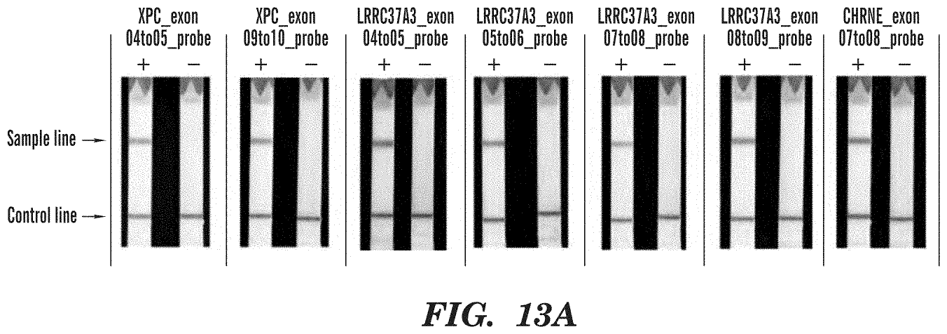

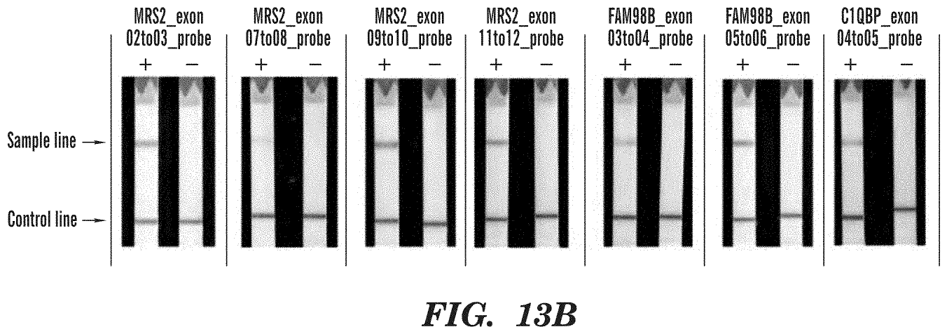

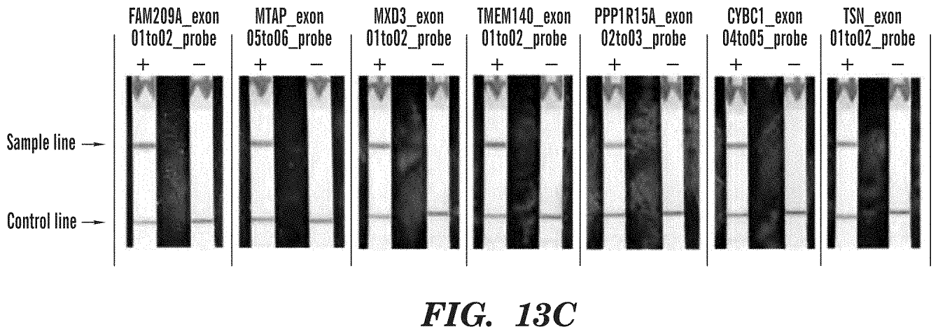

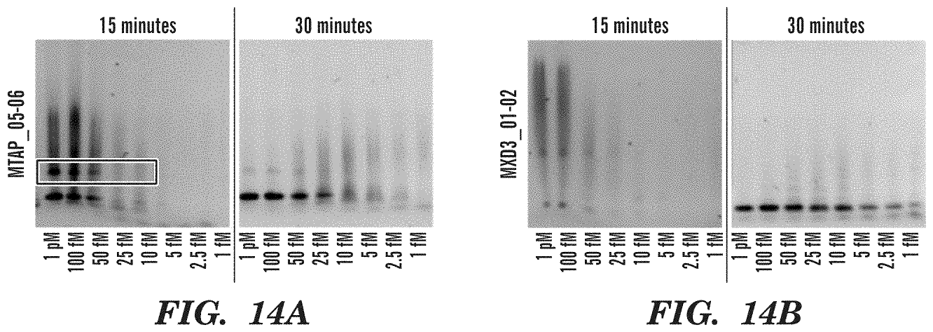

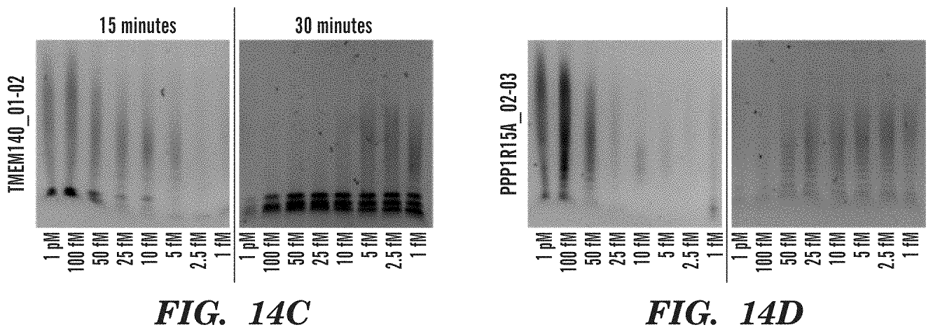

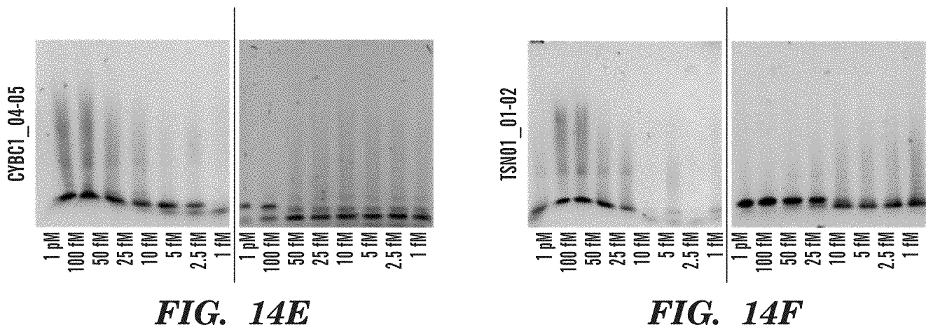

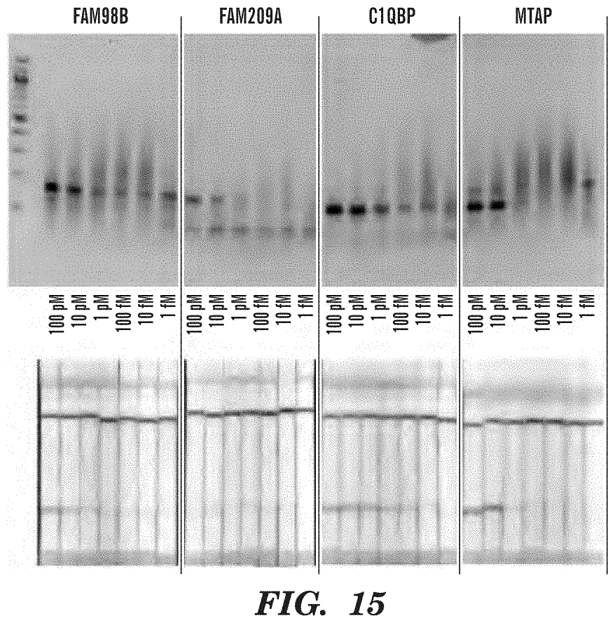

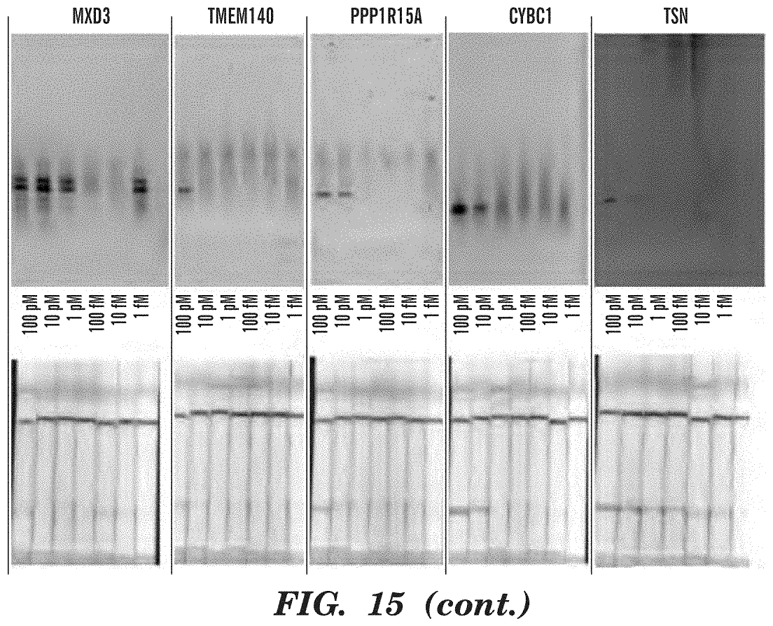

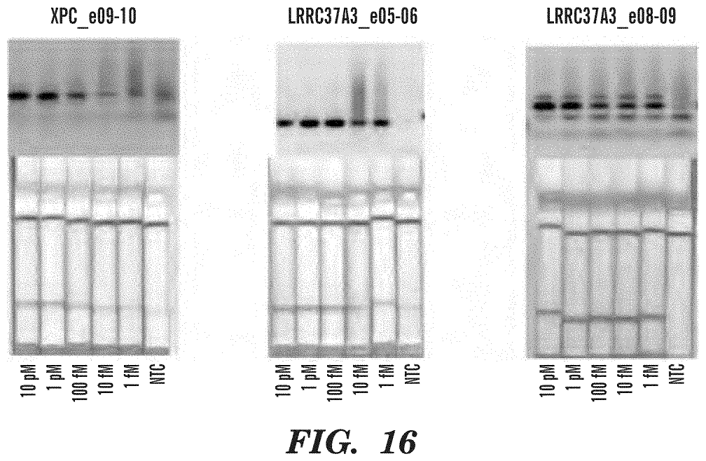

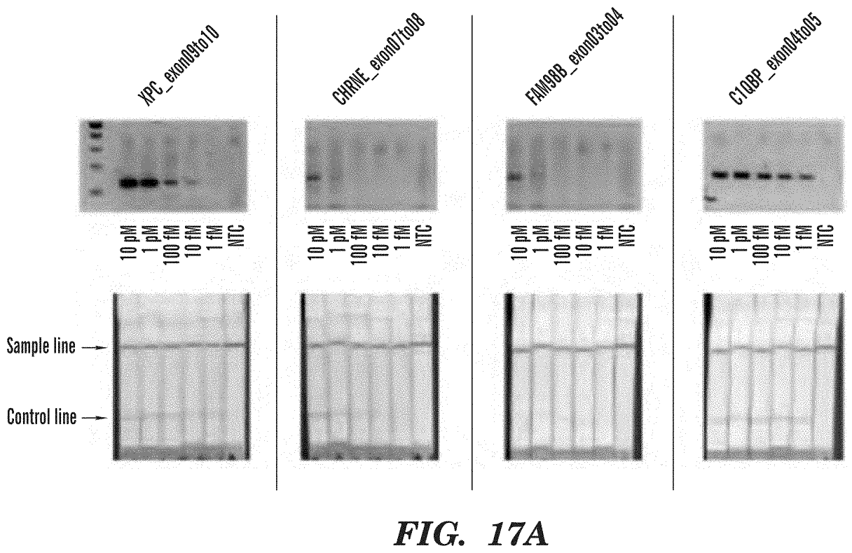

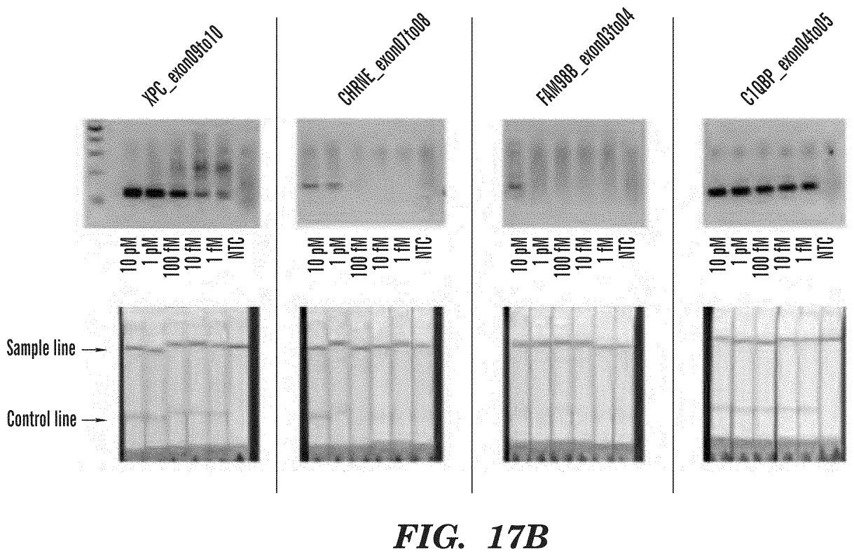

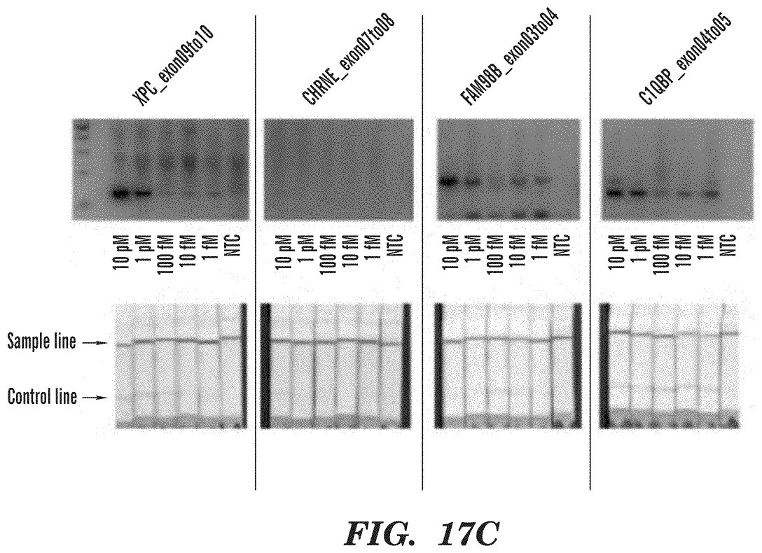

In some cases, it would be beneficial to have an assay designed without the constraint of needing primers with modifications. To demonstrate further robustness of the RecA readout-strategy, an RT-LAMP assay was designed using primers without any modifications. In this system, a set of probes was designed, each with a homology sequence to the dsDNA region of the LAMP dumbbell structure. With this scheme, RT-LAMP may be run without the need for a set of biotinylated primers, and, using the innate dsDNA structure of LAMP amplicon, the set of RecA probes are able to create filaments with the amplicon (FIG. 12A). When the RT-LAMP assay and probe/RecA incubation are ran in a two-pot reaction, the assay is sensitive down to 10 copies of nucleocapsid target Ref2A/reaction on paper (FIG. 12B-12C).

The capacity to use the strand-exchange activity of RecA for sensitive, specific detection of SARS-CoV-2 RNA using isothermal reactions suggests that it is possible to have a nucleic acid test for SARS-CoV-2 in under 15 minutes, with a sensitivity down to 6 copies of RNA/reaction.

### 2. Recombinase-Based Diagnostic Assay for Biomarkers Correlated to Radiation Exposure

In further embodiments of the disclosed technology, the RecA-probe-filament complex may be configured to detect a portion of a biomarker sequence that is correlated to radiation exposure. For example, the experiments that follow describe development of a point-of-care (POC) biodosimetry test for the measurement of biological response as a surrogate for radiation dose.

### 3. Recombinase-Based Sandwich Assay for Molecular Barcodes

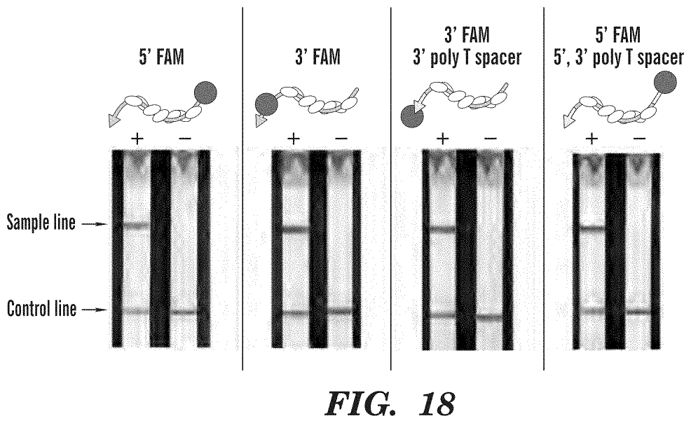

It is expected that, in certain usages of the disclosed technology, it is beneficial to have ssDNA probe sequences modified at either 5′ or 3′ without influence on RecA-based DNA binding. To examine the efficacy of varying the location of the ssDNA probe modification, a FAM moiety was placed at both the 5′ and 3′ ends of the probe, and iterations of the probe were designed such that a spacer sequence was placed between the modification and sequence homology region (FIG. 18). Signal intensity was not significantly impacted by varying the modification site, and an insertion of a spacer domain does not show influence on exchange of complementary base pairs promoted by RecA.

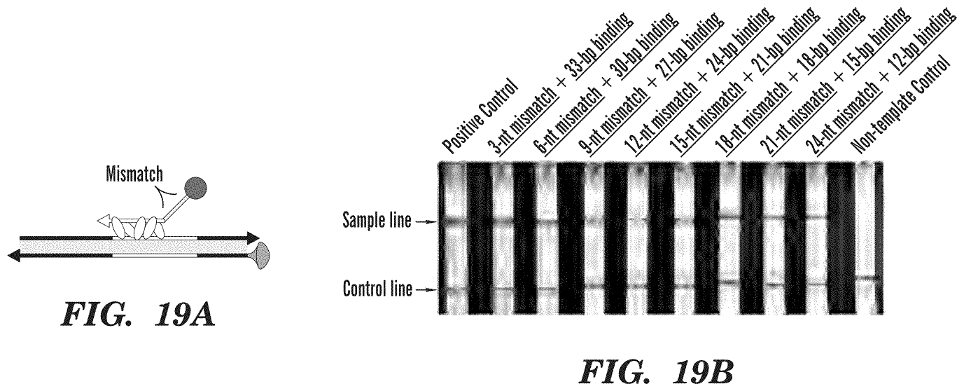

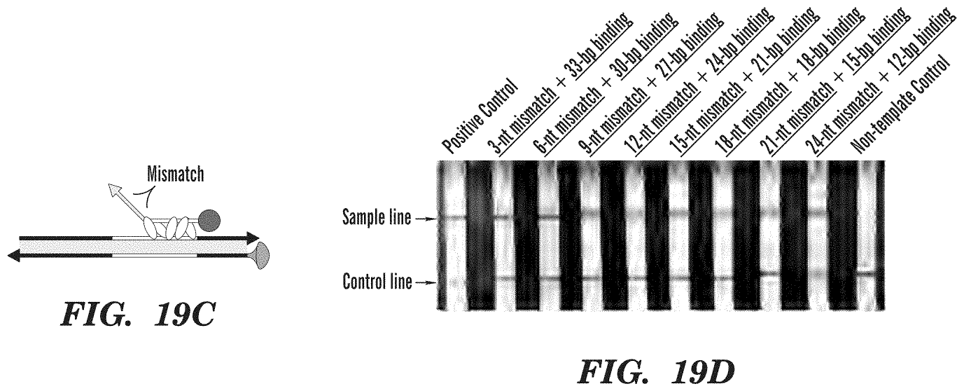

In various strand-exchange conformations, it is important to consider whether a mismatch of 3 nucleotides to 24 nucleotides at both the 5′ and 3′ end of the ssDNA probe sequence significantly impacts LFS signal intensity. The template binding domain to the RecA probe was gradually truncated from 5′ (FIG. 19A) or 3′ (FIG. 19B) of the probe to challenge the minimal probe length requirement for a RecA-based DNA binding. 12-bp ssDNA probes are eligible to recruit RecA enzyme and catalyze the strand exchange of complementary base pairs with homologous dsDNA (FIGS. 19A and 19B).

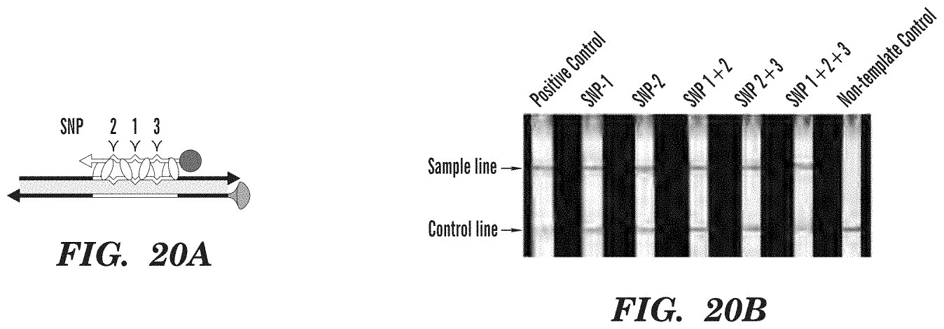

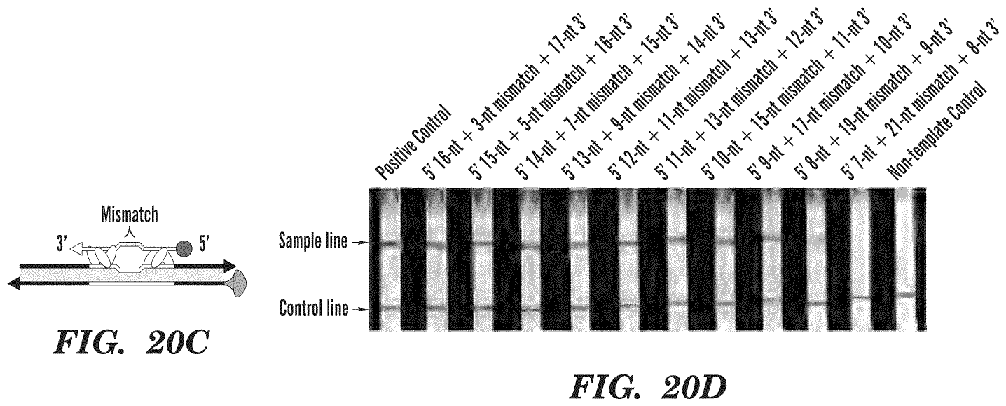

To further characterize the sensitivity of the proposed mechanism, an experiment was conducted to elucidate whether RecA-based dsDNA/ssDNA filament formation is sensitive to single nucleotide mutations occurring at the site of homologous dsDNA strand exchange. When synthetic dsDNA sequences were computationally designed and synthesized, RecA-based DNA binding was found insensitive to single nucleotide mutation occurring at homologous dsDNA (FIGS. 20A and 20B). Furthermore, when the mismatch region within a probe sequence was expanded from three nucleotides to 21 nucleotides within a 36-nucleotide long probe, RecA filament formation was strong for complementary regions greater than 8 nucleotides (FIGS. 20 C and 20D). RecA-based strand exchange flanking the 5′ and 3′ end of homologous DNA is sufficient to allow filament formation; when the homology region drops below 8 nucleotides to 7 or 6 nucleotides in length, no filament can form (FIG. 20D).

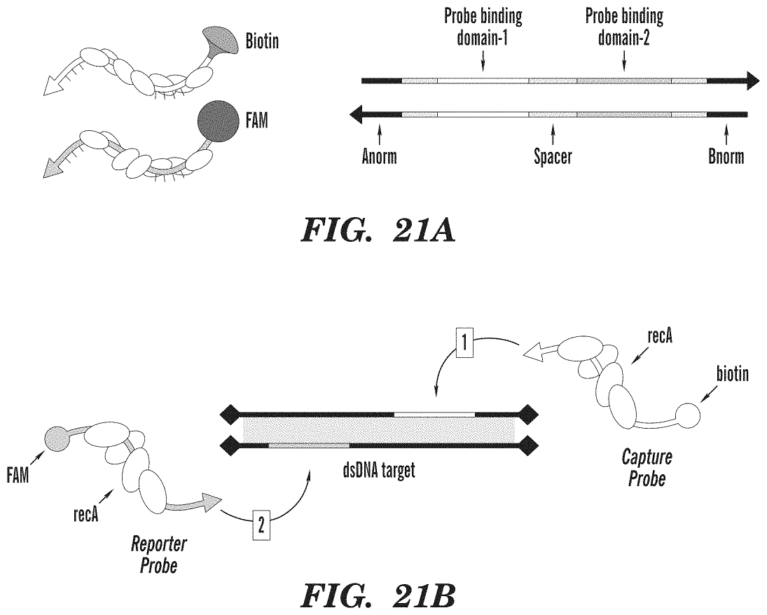

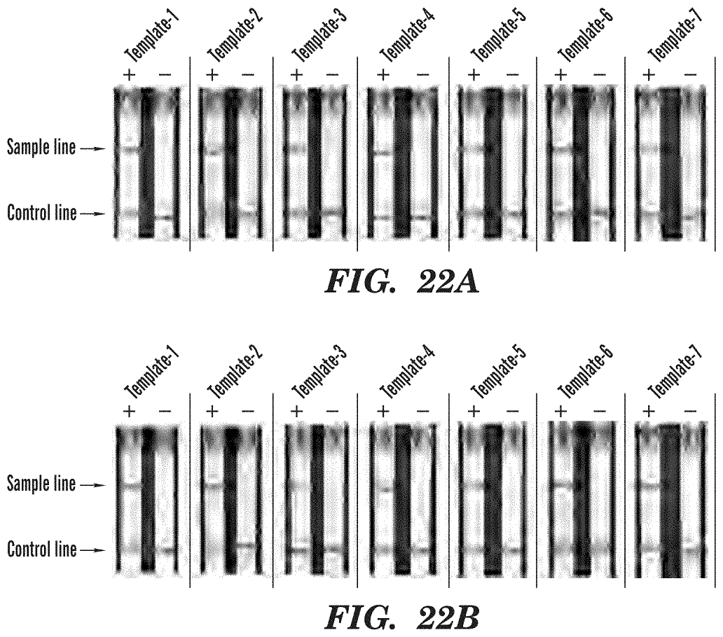

Additionally, the RecA-based detection mechanism can further comprise a scenario when both FAM- and biotin-labeled DNA may be used for targeting different regions of the same DNA strand, like targeting unmodified RT-LAMP (FIGS. 12, 21, and 23). To verify that multiple sites on a dsDNA strand may be targeted the RecA based detection mechanism and have strong signal when observed on a LFS, 7 unique dsDNA templates with either FAM- or Biotinylated-probes were tested. For each of the 7 unique dsDNA templates, the same probe was examined with varying modification (FIG. 22). All 7 targets were able to form filaments with the ssDNA probes and RecA, resulting in strong signal on a LFS.

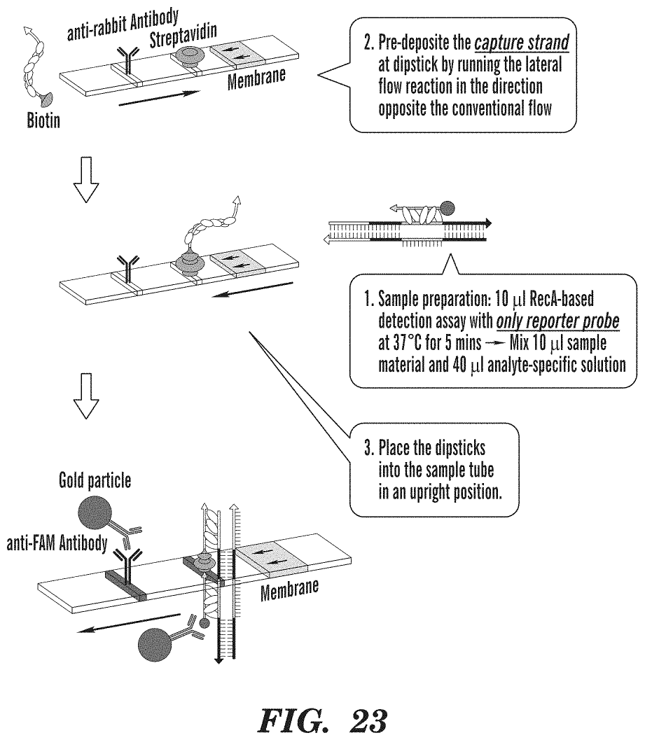

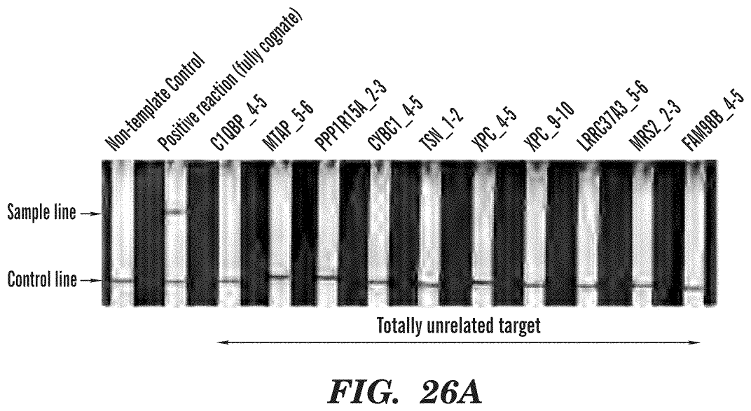

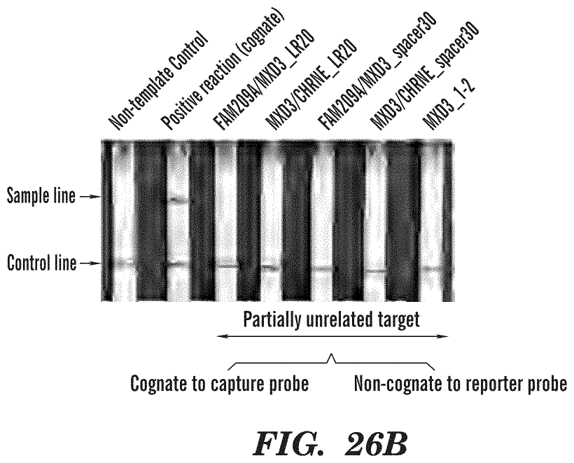

In an alternative application of the technology, ssDNA half barcodes first deposited on a lateral flow strip can use RecA to bind to dsDNA generated from isothermal amplification (FIG. 23). The biotin-containing capture probe can be pre-deposited to the sample line on the lateral flow strip. When the reporter probe/RecA/dsDNA template complex flows through the sample line, an in situ RecA-based DNA binding event can immediately occur to promote the formation capturer/reporter/RecA/dsDNA template complex. The connection between the FAM-containing reporter strand and the biotin-containing capture strand results in the form of a strong test band on the lateral flow strip (FIG. 26).

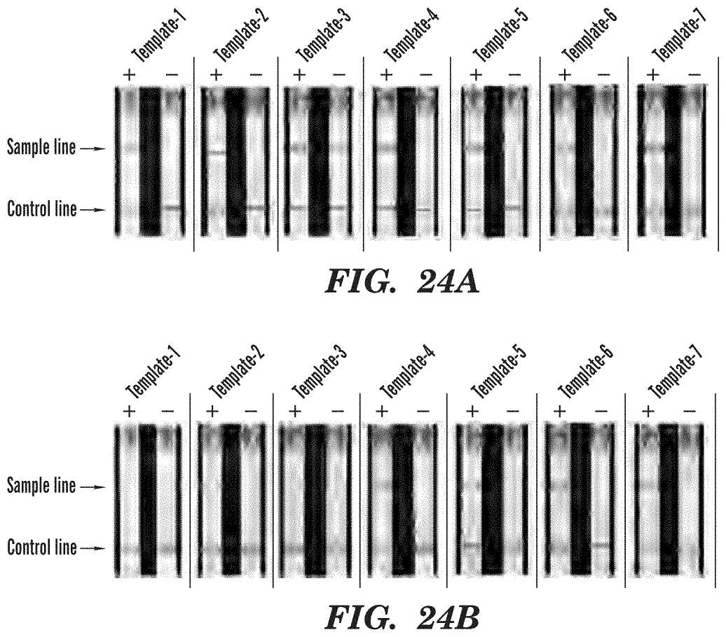

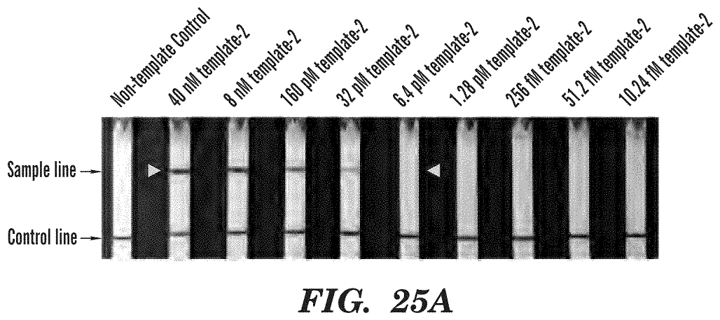

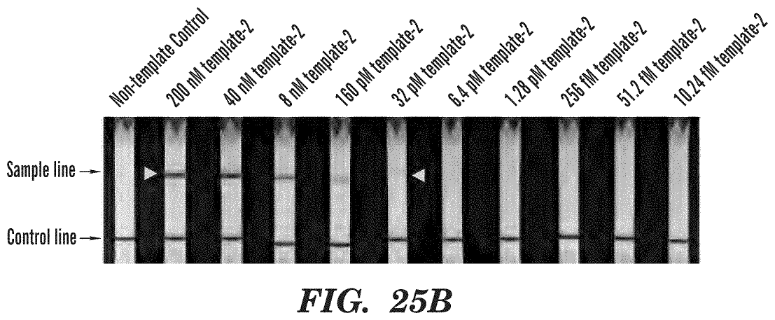

The detection limit of dsDNA target without pre-amplification of DNA target sequences. A conventional sandwich assay enables detection as low as 6.5 picomolar of dsDNA target (FIG. 25A). An in situ RecA-based sandwich-style assay on lateral flow strip (described in FIGS. 23 and 24) enables the detection as low as 160 picomolar of dsDNA (FIG. 25B).

For specific testing, RecA-based strand exchange must prove to be orthogonal across sequences, both in totally unrelated target sequences or with partially unrelated sequences. A specificity test of the RecA-based sandwich-style dsDNA targets prove the ability to specifically test only fully cognate target sequences, as no signal was observed by 10 unrelated dsDNA templates in a sandwich-type detection assay, indicating there is no false-positive probe binding (FIG. 26A). For 5 dsDNA templates that are only homologous to capture probe but not reporter probe, no signal on the sample line was observed, which indicates a good specificity of the RecA-based sandwich-type detection assay (FIG. 26B).

### 4. Recombinase-Based Strand Displacement Strategy

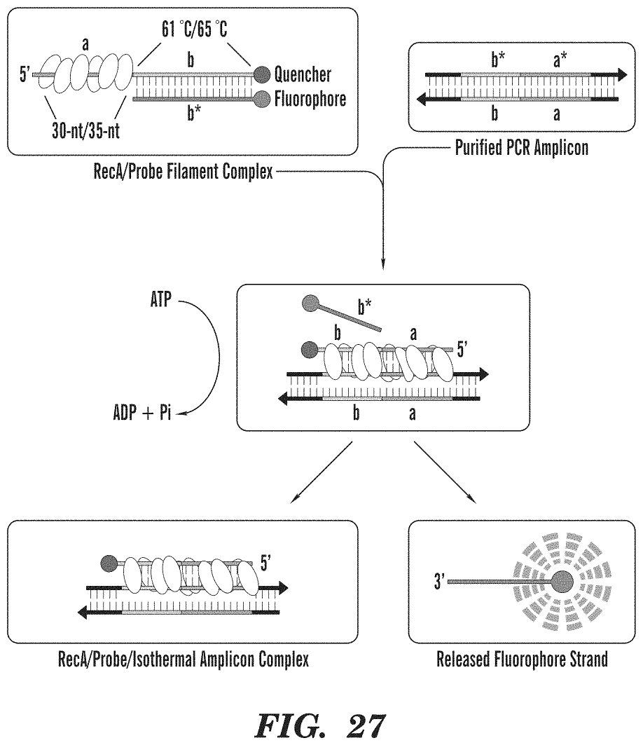

The E. coli RecA filament forms most readily on ssDNA and promotes the reactions that are typical for this class of protein. If the filament forms on linear ssDNA, DNA-strand invasion is promoted in which a 3′ end of the ssDNA invades a homologous duplex. The RecA probe complex to provide a fluorescent readout is composed of a short reporter, modified with a fluorophore, and a longer capture probe which has a reversely complementary region, and is modified with a quencher (FIG. 27). The single-stranded region of the capture probe must be 30 nucleotides for recruitment of RecA enzyme and formation of a filament to further initiate a RecA-based strand displacement. Binding between the RecA/DNA probe complex and the amplification product (dsDNA) eventually causes release of a fluorophore-containing reporter strand (FIG. 27).

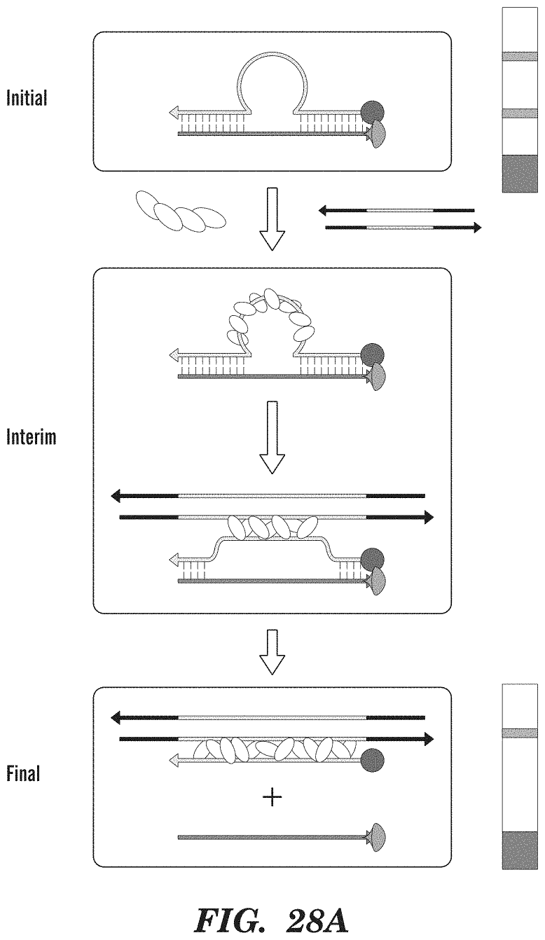

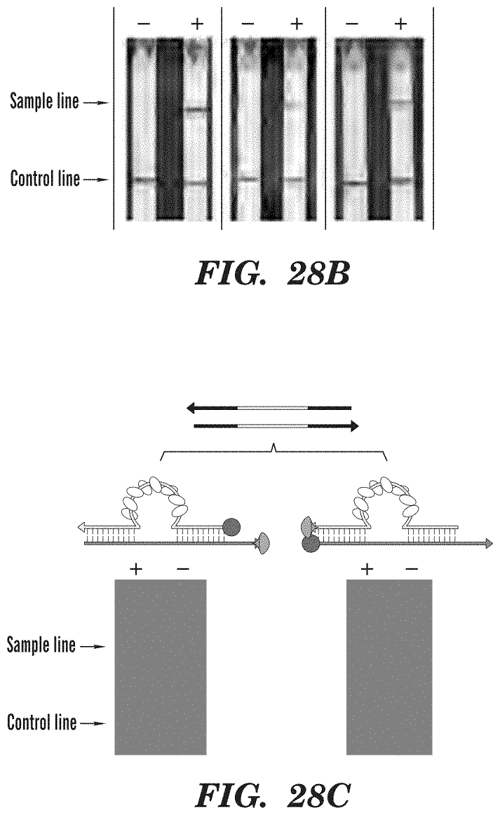

RecA-based strand displacement for the detection of target nucleic acids using a loop-mediated homology recognition site. The RecA probe complex is composed of a short biotin-containing reporter strand and a FAM-containing long capture strand which is partially based paired to reporter strand. In this scheme, the RecA enzyme forms a helical filament on the capture probe's central region mismatching from the reporter strand and promotes an exchange of complementary base pairs with the target dsDNA (FIG. 28A). The connection between capture probe and reporter probe forms a strong test band on the lateral flow strip (FIG. 28B), but when the biotin-containing reporter is released through a RecA-based strand displacement, the result is the absence of a band shown on lateral flow strip (FIG. 28C).

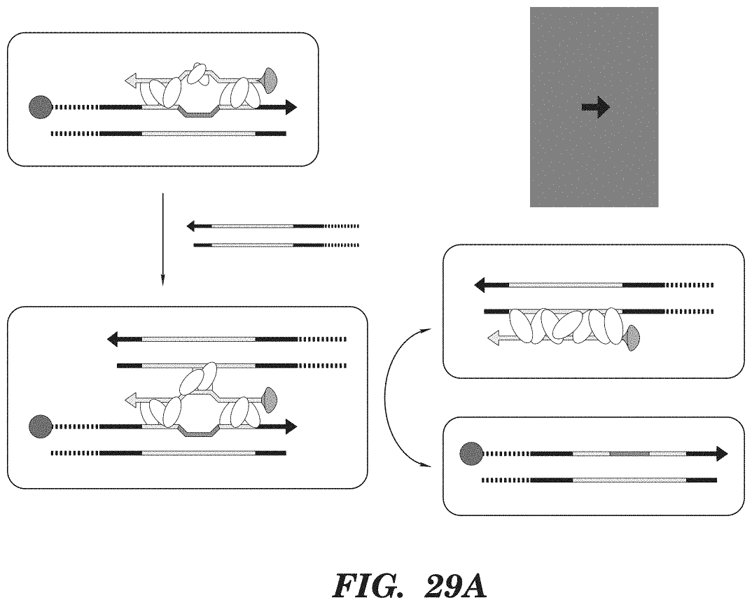

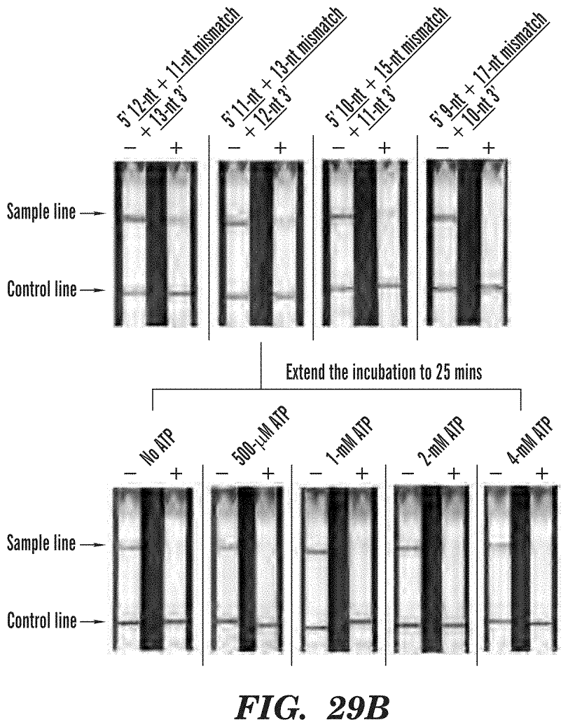

In further confirmations, RecA-based strand displacement can adopt a pre-formed dsDNA/ssDNA RecA filament complex (FIG. 29A). A biotin-containing ssDNA reporter probe can bind to a FAM-containing dsDNA probe through a RecA-based heteroduplex. The connection between capture strand and reporter strand would form a strong test band on the lateral flow strip (FIG. 29B). The RecA enzyme in a reaction can form a helical filament on the capture probe's central region, resulting in further mismatching from the reporter strand and promote an exchange of complementary base pairs with the target dsDNA. Through this mechanism, the biotin-containing reporter will be released and result in no band shown on the lateral flow strip because of the disruption of the reporter strand/capture strand connection (FIG. 29B). By extending incubation period from 10 minutes to 25 minutes and through the addition of ATP to the reaction, RecA-based strand displacement improves between the original three-strand filament and the dsDNA target.

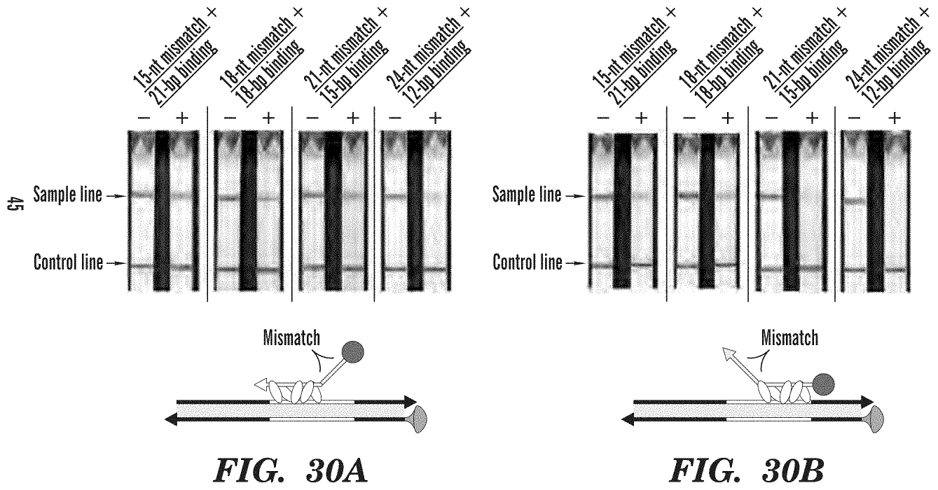

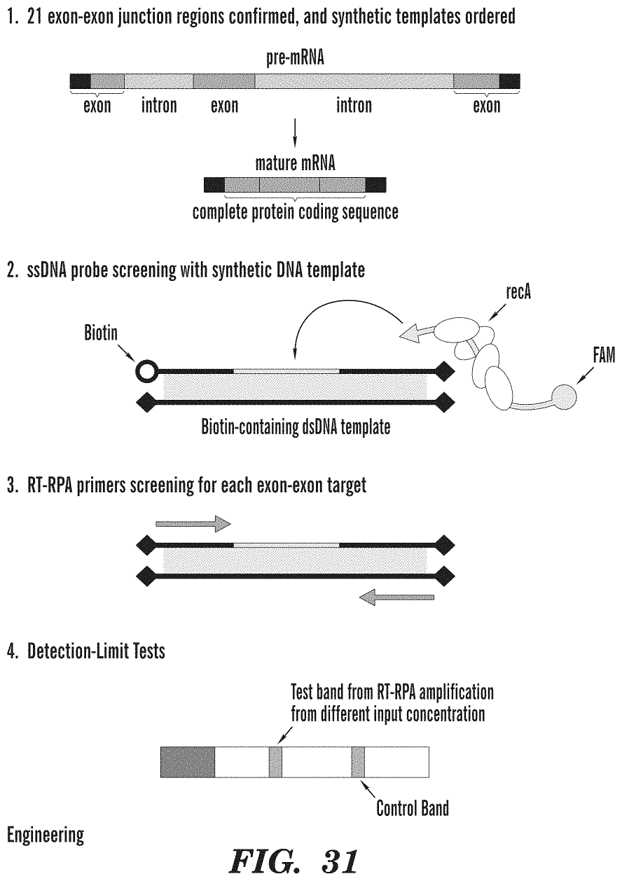

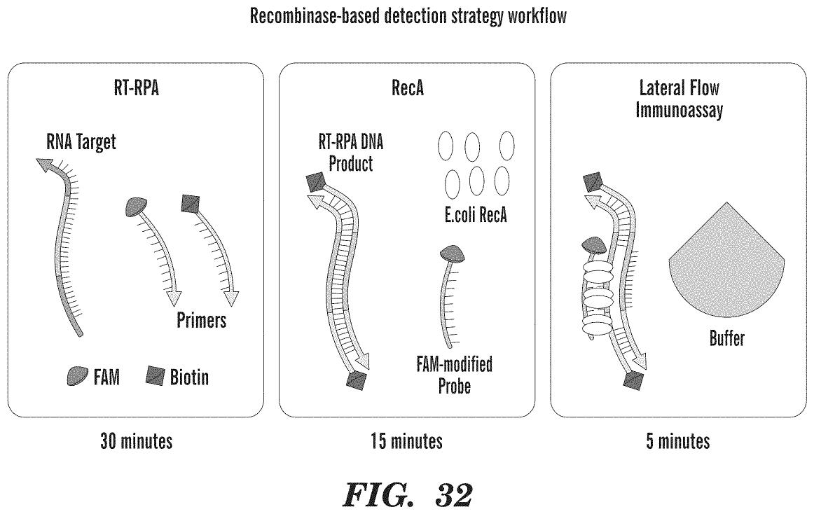

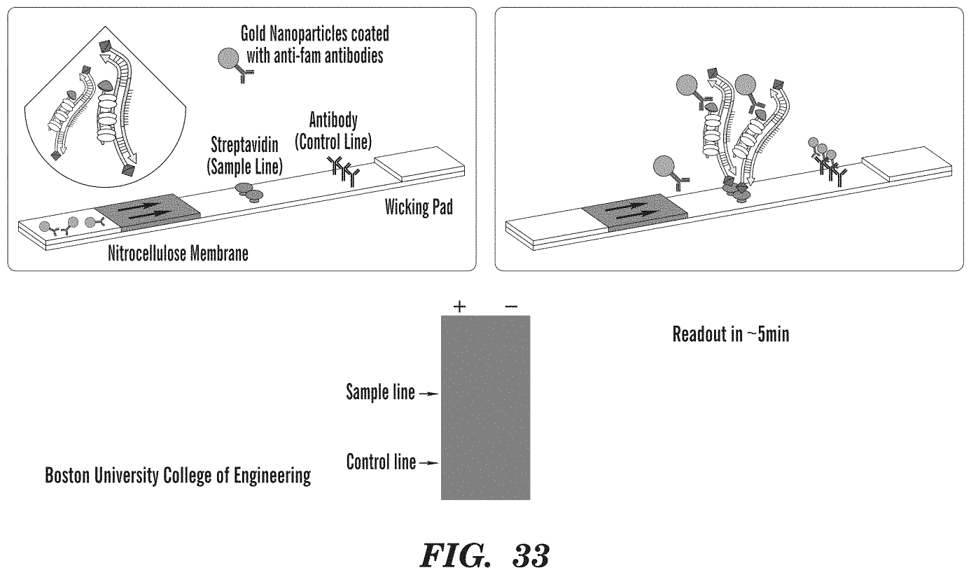

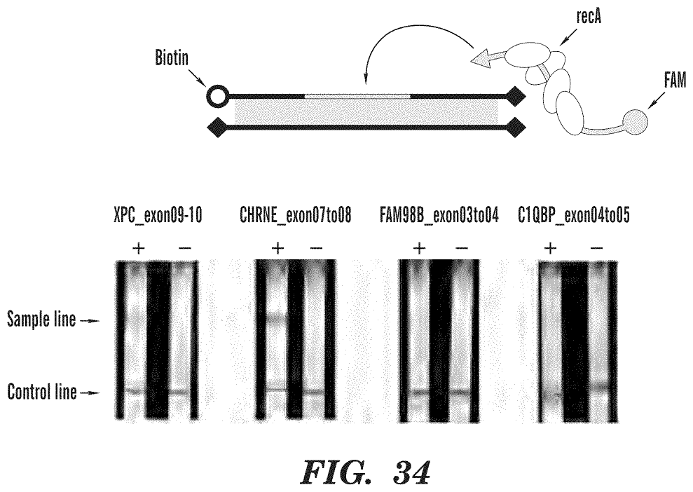

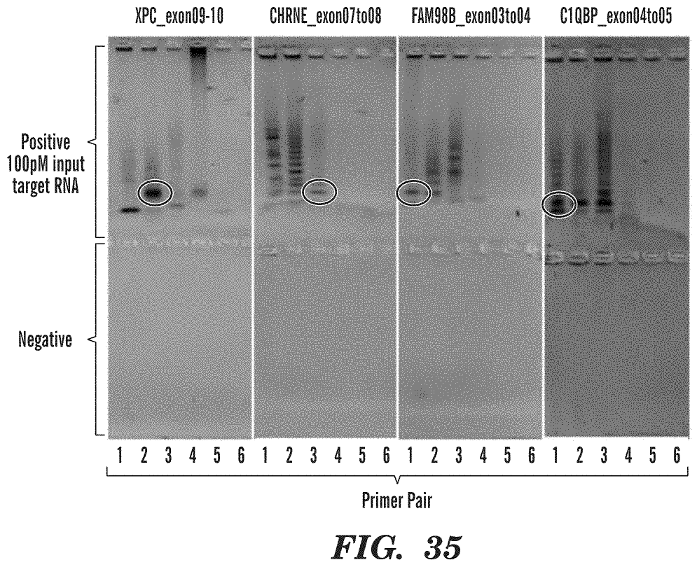

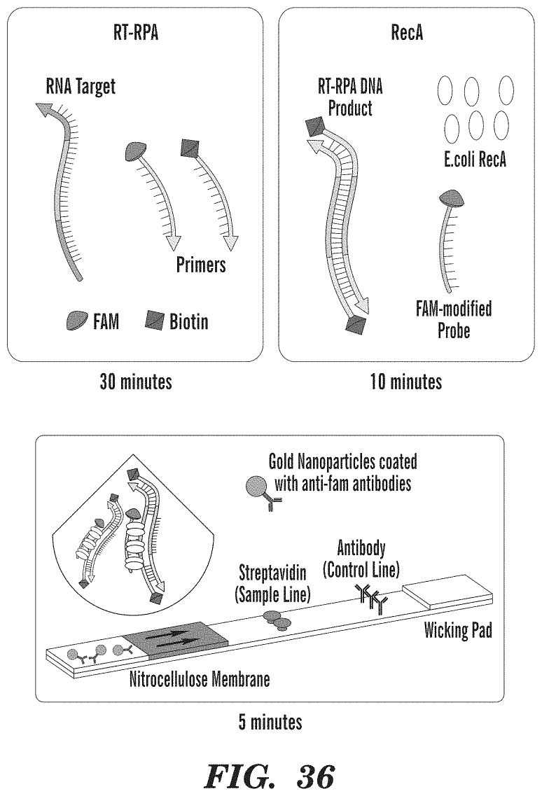

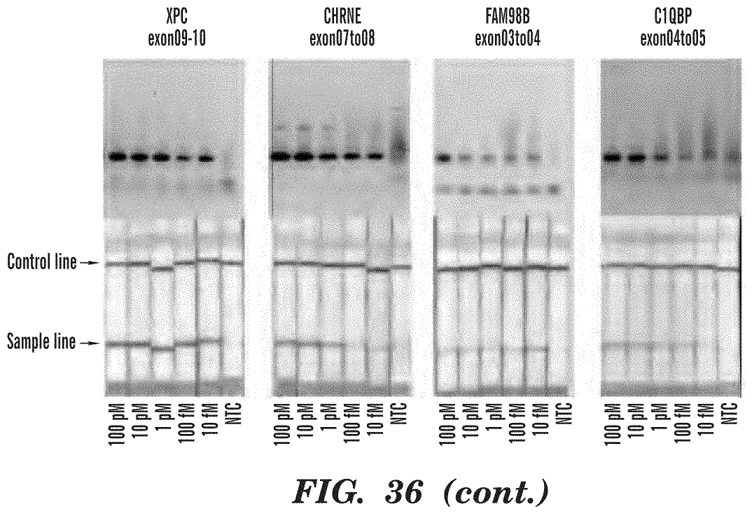

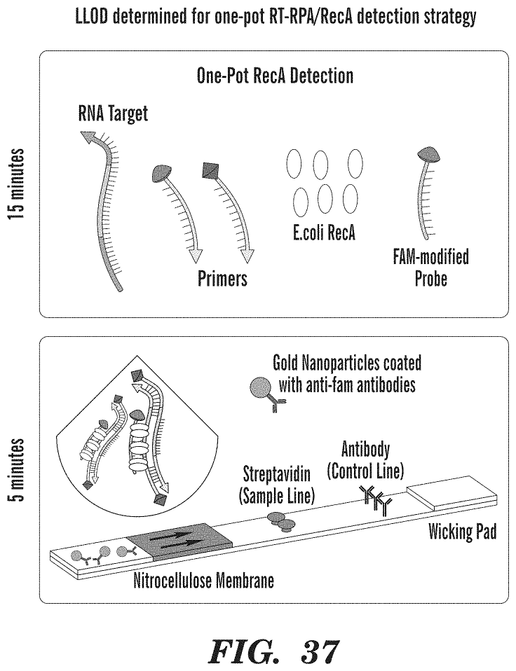

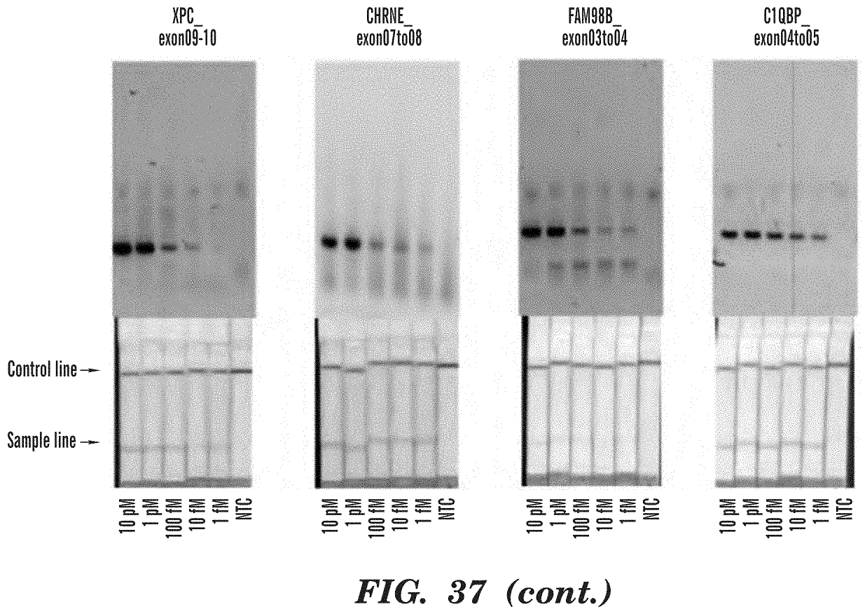

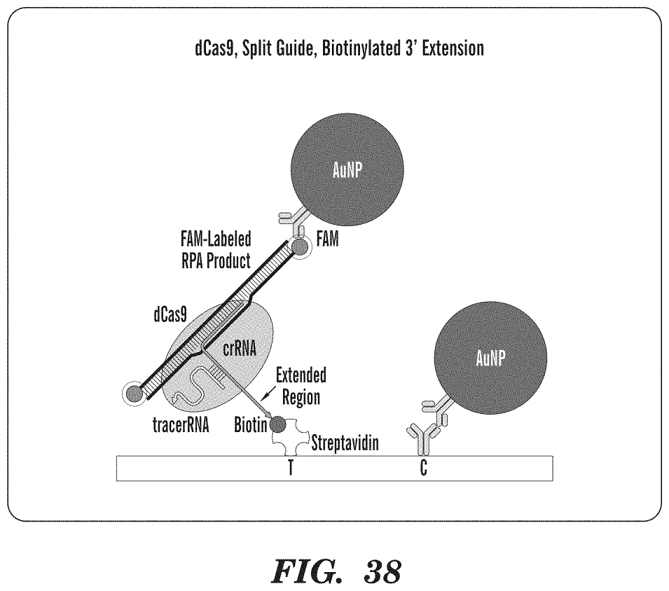

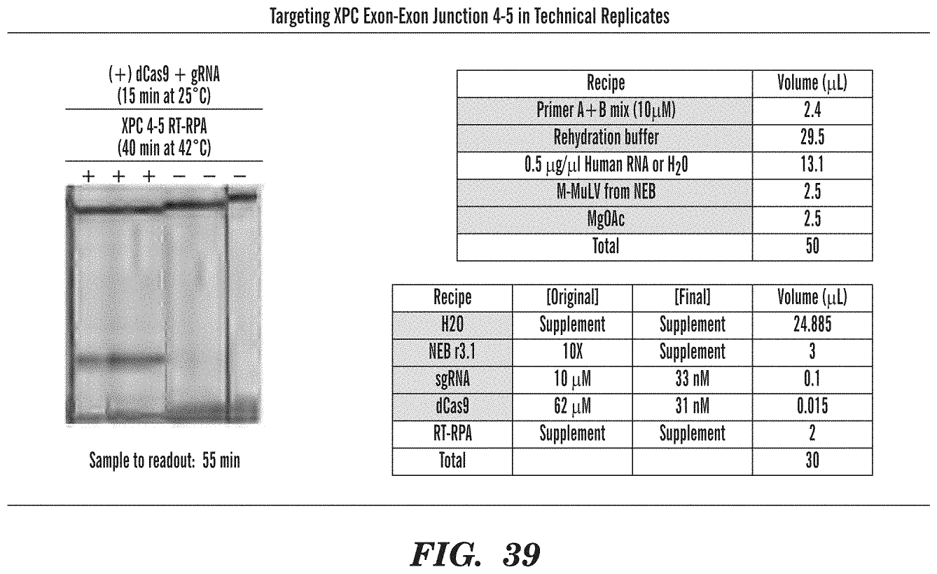

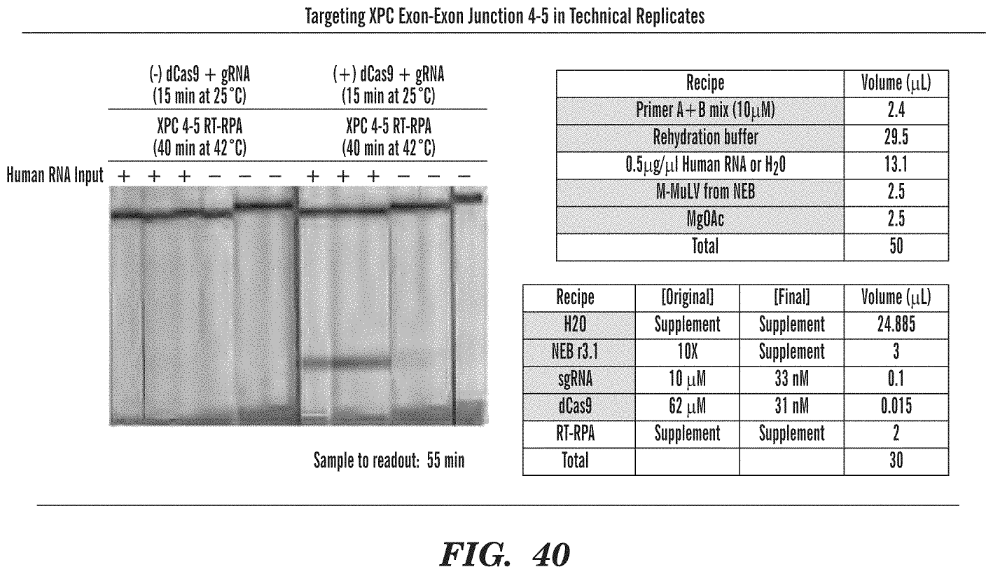

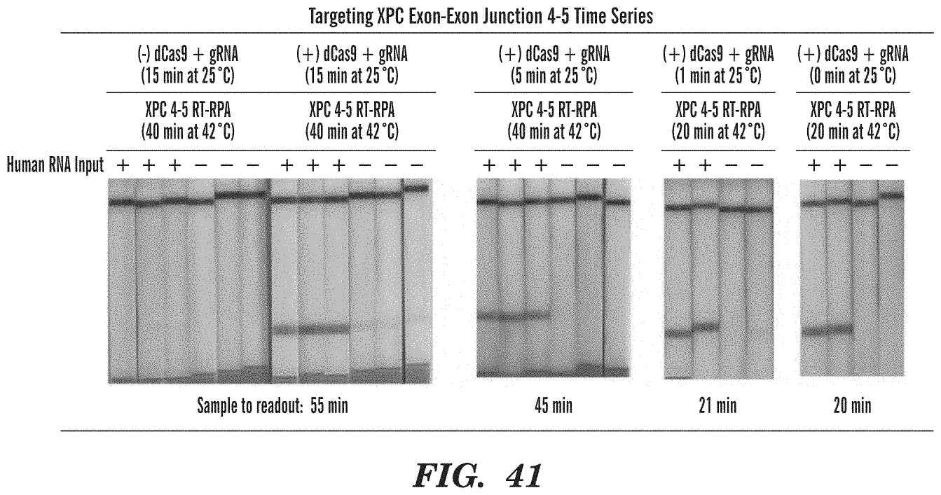

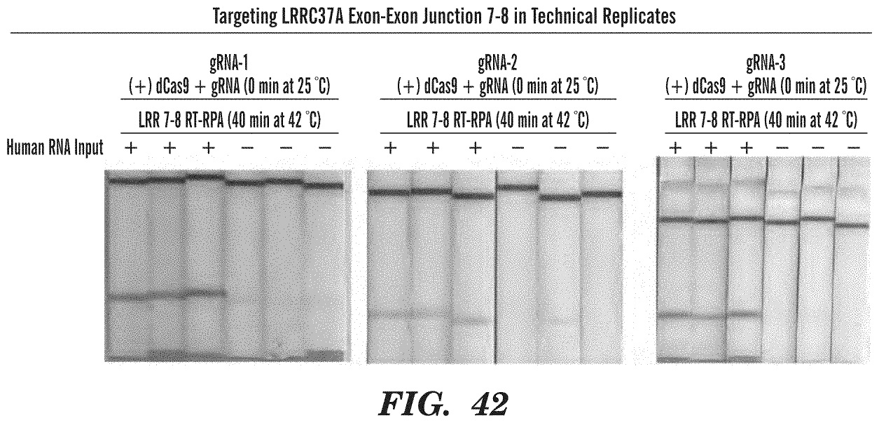

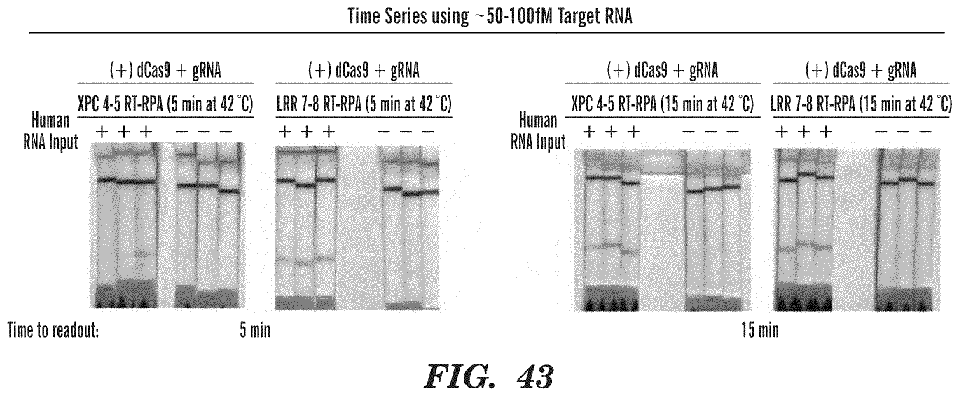

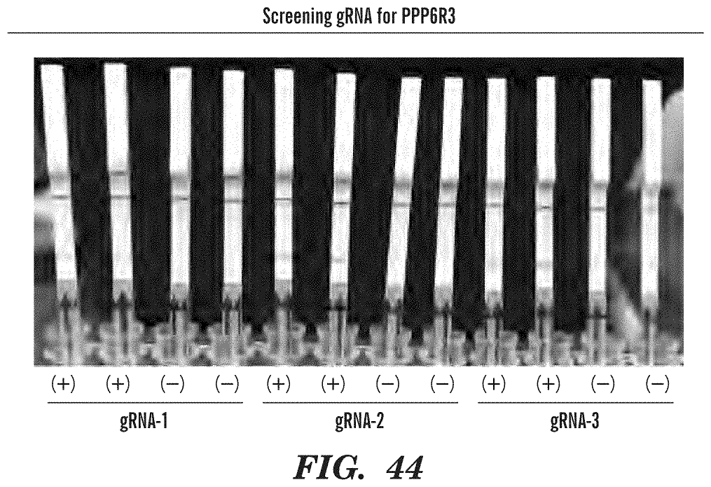

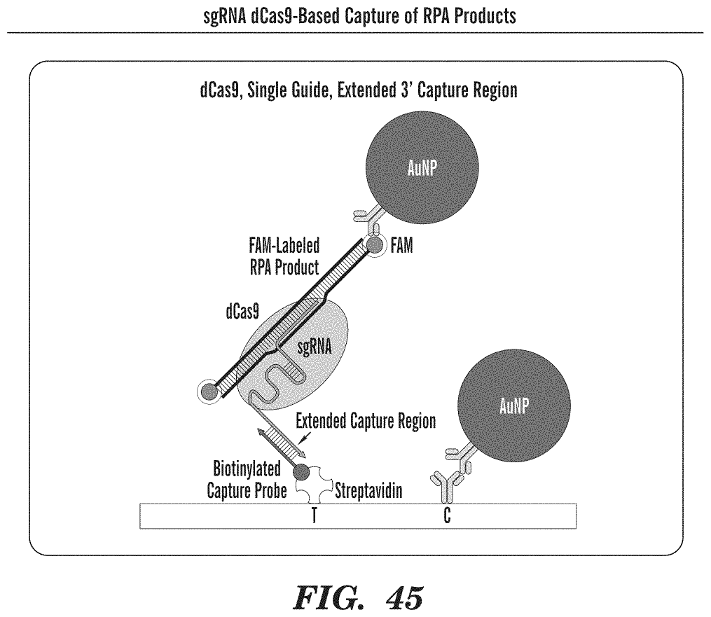

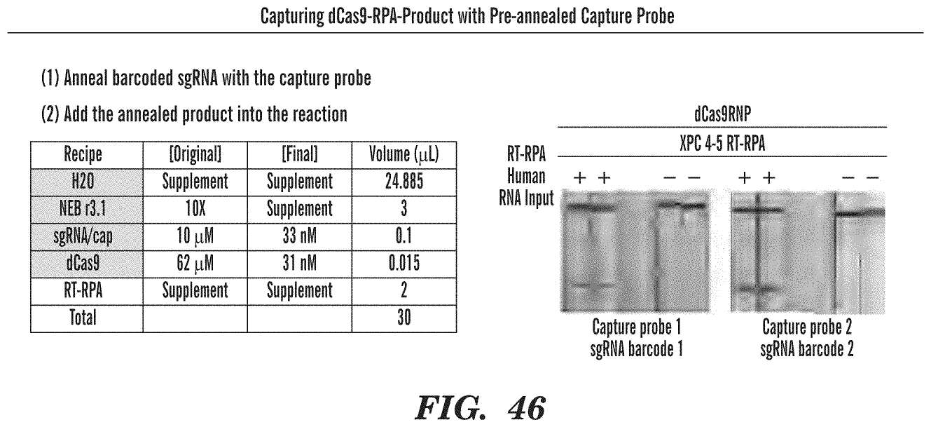

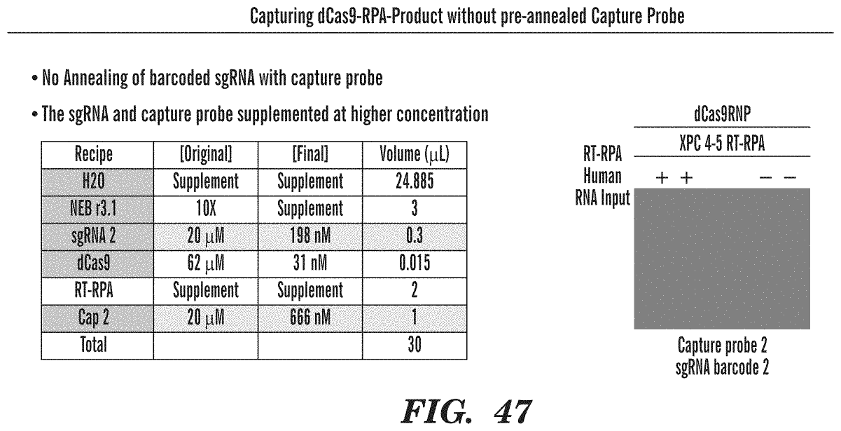

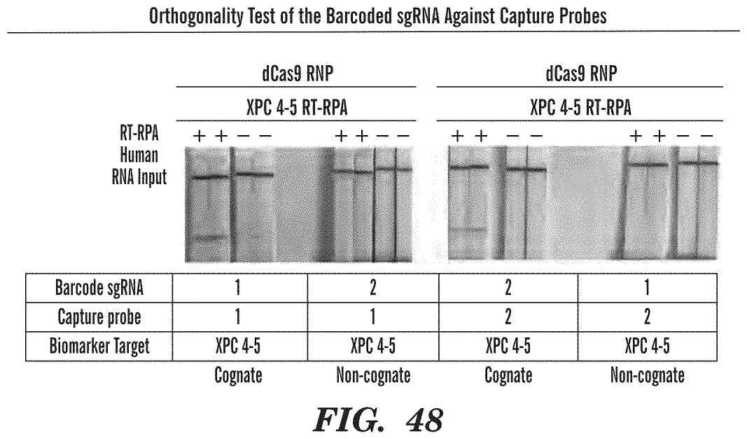

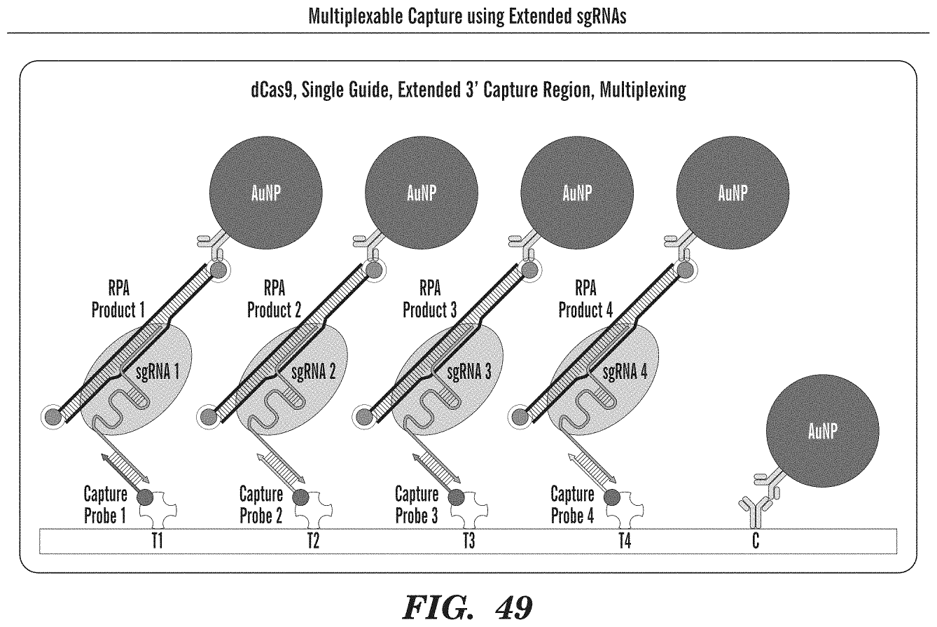

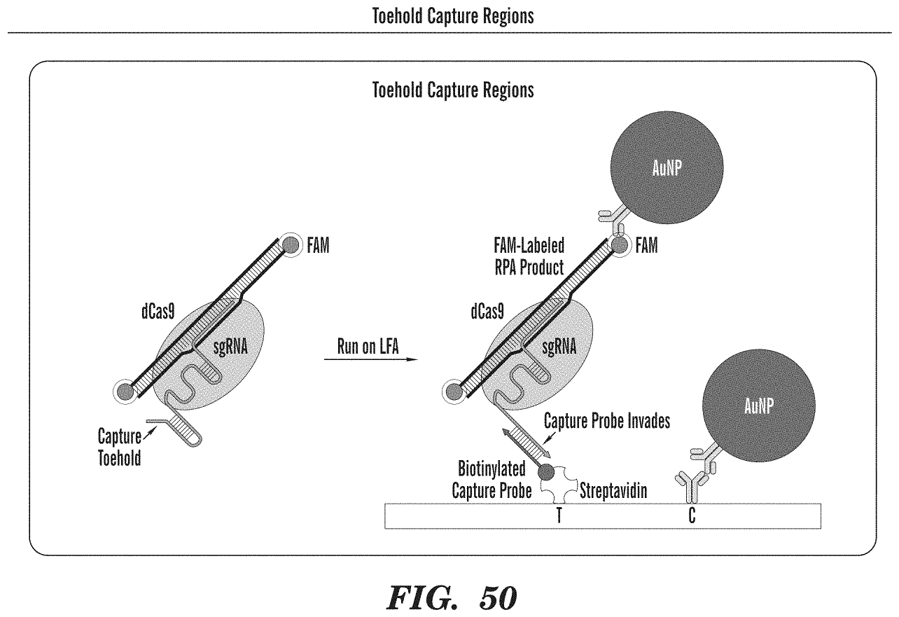

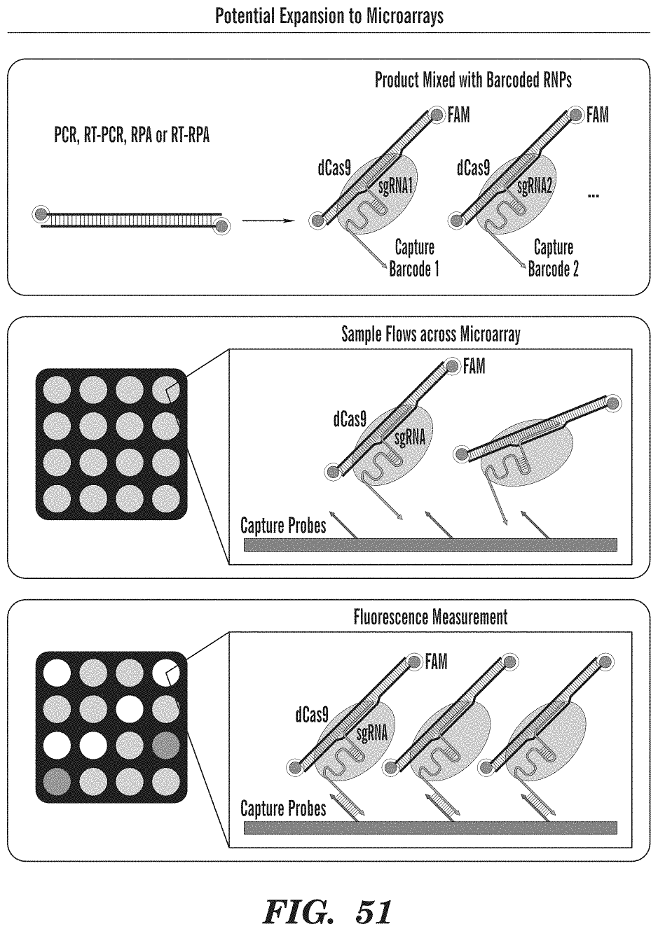

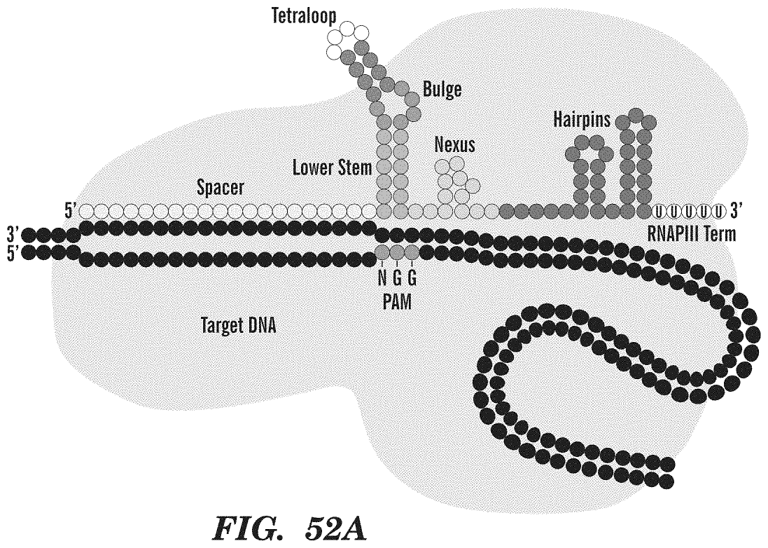

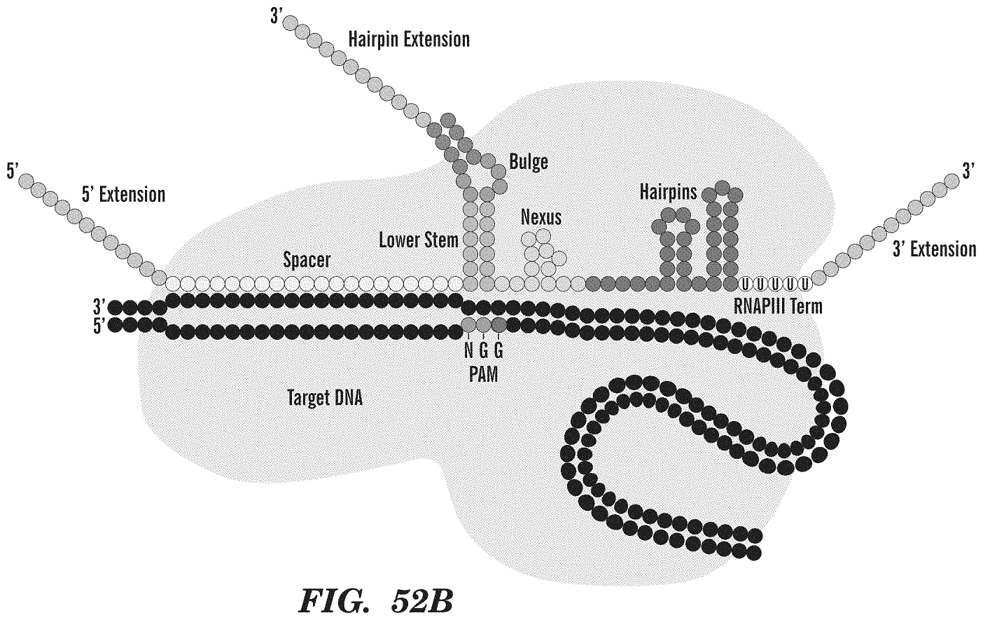

To elucidate whether the location of the homology region and proximity to the FAM modification impact LFS band intensity and performance, a series of mismatches were introduced on the 5′ end (FIG. 30A) and 3′ end (FIG. 30B) of a three-strand RecA filament. When the 3′ toehold of the reporter strand reaches 24-nucleotides, the band intensity difference from the lateral flow strip sample line could be observed, and results in visibly less LFS band intensity.

There are a variety of RecA proteins from different organisms, potentially with improved homologous recombination properties and/or temperature stabilities (e.g Taq RecA).

This technology can be used for implementing molecular barcode systems. Molecular barcodes are used for verifying the authenticity of different manufactured items, raw materials, etc., to prevent the dissemination of counterfeit goods. The RecA strategy, using DNA molecules as the molecular barcodes, could be used in this application area and provide fast results.

Although most of the results in this disclosure focus on optical readouts (e.g. visible lateral flow lines, fluorescence), modifications to the chemistry or sequence of the probe could yield other forms of readout. These include electrochemical signals, catalytic signals (e.g. a deoxyribozyme that cleaves a reporter substrate), translational or transcriptional signals, and the exposure of nucleic acid sequence to allow binding to other elements.

### Example 2: A Method of Detecting a Target RNA or DNA Molecule Containing a Homologous Domain

In certain embodiments, the method for detecting a target homologous RNA or DNA molecule in a sample comprises (a) contacting a RecA probe to a sample, where the probe is a synthetic nucleic acid molecule comprising at least one binding domain comprising a nucleotide sequence substantially complementary to at least a first portion of the amplicon, and a reporter molecule capable of producing a detectable signal; (b) performing an isothermal amplification to selectively amplify the target barcoding sequence-containing RNA or DNA molecule using a reverse transcriptase, DNA polymerase and monomers. In some cases, the homologous RNA or DNA is firstly amplified by isothermal amplification, for example, reverse transcription loop-mediated isothermal amplification (RT-LAMP) and reverse transcription-recombinase polymerase amplification (RT-RPA); and (c) lateral flow device comprising a capture/test region capable of localizing a recA probe/target molecule complex. Where the homologous domain of target RNA or DNA is a pathogen-associated nucleic acid biomarker, detecting a homologous RNA or DNA in vitro is a positive or negative indicator of a pathogen infection.

### Method-1: Single Target Detection Assay Through RecA-Assisted DNA Binding and Isothermal Amplification

1.1 Two Step Assay (Isothermal Amplification->RecA-Based DNA Binding)

Step-1: A target RNA or DNA molecule is firstly isothermal amplified, for example, RT-LAMP or RT-RPA, through 3 or 1 pair of primers comprising the FAM or biotin moiety, to make target modified with an additional FAM or biotin moiety in double stranded DNA format which is subjected to the ssDNA probe/RecA filament complex.

Step-2: A ssDNA probe, modified with either biotin or FAM reporter moiety reacts with RecA enzyme first to create a filament complex. The ssDNA probe comprises at least one single-stranded homology domain which is complementary to a homologous target RNA or DNA, or the reverse complement. The reaction between probe/recA filament and the product from step-1 results in the formation of a triple-stranded filament yielding the connection of FAM and Biotin molecules.

Detection of the triple stranded RecA filament is conducted on a lateral flow strip with a streptavidin sample line and colloidal gold particles coated with an anti-FAM antibody. When the immunochromatographic assay is run, binding of the gold nanoparticle allows for visual detection of either the modified dsDNA target or ssDNA probe.

1.2 One-Pot Assay (Isothermal Amplification and RecA-Based DNA Binding Occur in the Same Reaction)

Thermus aquaticus RecA protein (65˜75° C.) enables this RecA-based diagnostic assay coupling with isothermal amplification spanning different temperatures in a one-pot reaction.

Target DNA or RNA is first isothermally amplified through LAMP or RT-LAMP, respectively, using the primers that add a FAM or Biotin modification to the amplicon. RT-LAMP has an operating temperature of 61° C. to 71° C. and the use of Thermus aquaticus RecA protein (65˜75° C.) enables the integration of RecA-based detection and RT-LAMP in the same reaction.

During homologous recombination, RecA forms a helical filament on an ssDNA probe having Biotin or FAM modification that searches for a homologous dsDNA and catalyzes the exchange of complementary base pairs to form a new heteroduplex. The form of a triple-stranded complex with a homologous region in dsDNA enables the connection between FAM-having RT-LAMP amplicon and Biotin-containing probe (or biotin-having RT-LAMP amplicon and FAM-containing probe), which promotes sample band visualization on the lateral flow strip through the streptavidin/biotin/FAM/FAM-antibody/gold particle multi-layer labeling. A strong sample line is then used to indicate that the target RNA or DNA molecule is present in the patient sample.

Method-2: Multiplexed Testing Through RecA Probes with Dual Binding Domain

1.1 Customizable lateral flow device: multiple capture probes which are subjected to different reporter probes are first pre-deposited on the lateral flow device as sample lines for multiplexed profiling of different potential target nucleic acids.

1.2 A reporter probe is a synthetic nucleic acid molecule comprising (a) a target amplicon binding domain comprising a nucleotide sequence substantially complementary to at least a first portion of the amplicon; and (b) capture probe binding domain through base pairing.

1.3 Target DNA or RNA is first isothermally amplified through LAMP or RT-LAMP, respectively, using the primers that add a FAM modification to the amplicon. Use of E. coli or Thermus aquaticus RecA protein (6575° C.) enables either a two-step reaction or one-pot reaction.

1.4 The form of a triple-stranded complex with a homologous region in dsDNA amplicon enables the connection between FAM-having RT-LAMP amplicon and reporter probe. During the lateral flow reaction, the base pairing between capture probe and capture probe binding domain of reporter probe localize the complex on the lateral flow strip for sample band visualization through the capture probe/reporter probe/FAM-having amplicon/FAM-antibody/gold particle multi-layer labeling. A strong sample line is then used to indicate that the target RNA or DNA molecule is present in the patient sample.

### Method-3: Multiplexed Testing Through RecA-Based DNA Binding-Mediated Strand Displacement

### Example-1: Readout on LFS for Disrupting FAM/Biotin Connection

This method for detecting a target RNA or DNA molecule comprises a probe pair composed of a reporter strand and block strand, where two strands are partially base-paired on 5′ end and 3′ end and a mismatched loop domain is exposed on the central region of the block strand. Two strands contain an either FAM or biotin reporter molecule on either end, respectively, capable of producing a detectable signal on lateral flow device.

Two strands will be pre-annealed for promoting the connection between FAM and biotin molecules. In the absence of cognate target DNA or RNA molecule, a strong test band on the lateral flow strip indicates the absence of the target DNA. Once the target DNA presents, the binding between loop domain on block strand and target DNA through RecA promotes the catalysis of a DNA synapsis reaction between a DNA double helix and a complementary region of single-stranded DNA, which further catalyzes bidirectional branch migration to 3′ and 5′ till displace the reporter probe from block strand. The disruption of FAM-biotin connection results in the missing band on the sample line, indicating that the target DNA molecule is present in the patient sample.

### Example-2: Readout on LFS for Promoting FAM/Biotin Connection

This method for detecting a target homologous dsDNA molecule in a sample use a probe pair composed of a reporter-containing reporter strand and a block strand, where two strands are synthetic nucleic acid, are partially base-paired on 5′ end and 3′ end, and a mismatched loop domain which are substantially complementary to target dsDNA is exposed on the central region of the block strand.

An isothermal amplification is performed to selectively amplify the target barcoding sequence-containing RNA or DNA molecule using a reverse transcriptase, DNA polymerase and monomers. In some cases, the homologous RNA or DNA is firstly amplified by isothermal amplification, for example, reverse transcription loop-mediated isothermal amplification (RT-LAMP) and reverse transcription-recombinase polymerase amplification (RT-RPA), and biotin or FAM-containing primer offers the amplicon with modification molecule.

The reporter-containing reporter strand and a block strand will be pre-annealed for preventing unspecific binding between amplicon and reporter strand, providing no band on the sample line on a lateral flow device. Once the target DNA presents, the RecA-based binding between single stranded loop domain on block strand and complementary region of amplicon DNA helix promotes the catalysis of a DNA synapsis reaction, which further catalyzes bidirectional branch migration to 3′ and 5′ till displace the reporter probe from block strand. The connection of FAM and biotin from the reporter strand/RecA/amplion filament results in a strong band on lateral flow device, indicating that the target DNA molecule is present in the patient sample.

### Example-3: Read-Out by Fluorescence Through Loop-Mediated Strand Displacement

This method for detecting a target RNA or DNA molecule comprises a probe pair composed of a fluorophore-containing reporter strand and a quencher-having block strand, where two strands are partially base-paired on 5′ end and 3′ end and a mismatched loop domain is exposed on the central region of the block strand.

The quencher and reporter molecules are on the same side. Two strands will be pre-annealed for promoting the spacer approaching touch between fluorophore and quencher molecules. In the absence of cognate target DNA or RNA molecule, non fluorescence because of Förster resonance energy transfer (FRET) indicates the absence of the target DNA. Once the target DNA presents, the binding between loop domain on block strand and target DNA through RecA promotes the catalysis of a DNA synapsis reaction between a DNA double helix and a complementary region of single-stranded DNA, which further catalyzes bidirectional branch migration to 3′ and 5′ till displace the reporter probe from block strand. The disruption of FAM-quencher connection results in the activation of fluorescence, indicating that the target DNA molecule is present in the patient sample.

### Example-4: Read-Out by Fluorescence Through Toehold-Mediated Strand Displacement

This method for detecting a target RNA or DNA molecule comprises a probe pair composed of a short fluorophore-containing reporter strand and a long quencher-having block strand, where two strands are base-paired on either 5′ end or 3′ end of block strand and a linear toehold domain is exposed on either 3′ or 5′ of the block strand.

The quencher and reporter molecules are on the same side. Two strands will be pre-annealed for promoting the spacer approaching touch between fluorophore and quencher molecules. In the absence of cognate target DNA or RNA molecule, non fluorescence because of Förster resonance energy transfer (FRET) indicates the absence of the target DNA. Once the target DNA presents, the binding between toehold domain on block strand and target DNA through RecA promotes the catalysis of a DNA synapsis reaction between a DNA double helix and a complementary region of single-stranded DNA, which further catalyzes unidirectional branch migration till displace the reporter probe from block strand. The disruption of FAM-quencher connection results in the activation of fluorescence, indicating that the target DNA molecule is present in the patient sample.

### Method-4: Multiplexed Testing Through Sandwich-Style Binding of Duplex DNA Via RecA

This method for detecting a target homologous dsDNA molecule in a sample comprises (a) pre-depositing a capture probe, where the probe is a synthetic nucleic acid molecule comprising at least one binding domain substantially homologous to target dsDNA molecule, to the lateral flow device as a sample line for localizing the reporter probe/RecA/target dsDNA molecule; and (b) contacting a reporter probe to a sample, where the probe is a synthetic nucleic acid molecule comprising at least one binding domain comprising a nucleotide sequence substantially complementary to at least a first portion of the target dsDNA, and a reporter molecule, like FAM, capable of producing a detectable signal.

When the reporter probe/RecA/dsDNA template complex flows through the sample line with the cognate capture probe to target molecule, another in situ RecA-based DNA binding event can immediately occur to promote the formation capturer/reporter/RecA/dsDNA template complex. The connection between the FAM-containing reporter strand and the capture strand results in the form of a unique and strong test band on the lateral flow strip for reporting a pathogen-associated nucleic acid biomarker.

### Example 3: A Method of Detecting a Target RNA or DNA Molecule Using dCas9

Cas9 and dCas9 can use either a two-part (crRNA and tracrRNA) or single guide RNA (sgRNA); In the case of the two-part guide, the 3′ end of the crRNA lays outside of the cas9 enzyme and can be modified without impacting the functionality of the complex. Likewise, the 3′ end of the sgRNA can also be extended or modified with a capture moiety without impacting the RNP functionality. The addition of a capture moiety allows for direct capture of the guide RNA and by extension the Cas protein and any bound amplicons. Alternatively, by inserting orthogonal RNA sequences into either of these regions we can specifically capture RNPs of interest through base-pairing. Since this capture region is part of the same guide RNA as the target-determining spacer sequence we can easily pair targets with a respective capture sequence.

### Generation of FAM-Labeled RPA Product:

DNA amplicons were generated via Recombinase Polymerase Amplification (RPA) or Reverse Transcription Recombinase Polymerase Amplification (RT-RPA) using a TwistAmp Basic RPA kit (TwistDx), in the case of RT-RPA the kit is supplemented with 500 units of M-MuLV Reverse Transcriptase per 50 μL reaction. RPA primers are purchased pre-labeled with 5′FAM or alternative detection moieties (IDT) for later detection on lateral flow. The reaction mixture is prepared according to the manufacturer's specifications. The reaction is then incubated at 42° C., the required incubation time is target dependent but is typically 15-60 min. The reaction can be optionally stopped using a 80° C. heat inactivation step.

Biotinylated crRNA Configuration:

For the biotinylated crRNA configuration, crRNA with a spacer sequence targeting the DNA amplicon of interest was synthesized with a and a 3′ biotin modification (IDT). This was annealed to tracrRNA (IDT) at a concentration of 10 μM of each component in Duplex Buffer (IDT) by heating to 95° C. and allowing it to cool at room temperature until equilibrated. For amplicon capture, 2 μL of FAM-labeled RPA product is mixed with 31 nM dCas9 (IDT) and 33 nM annealed guide RNA in 1X NEBuffer r3.1 (NEB) in a 30 μL reaction mix. No incubation time is required before the addition of 70 μL lateral flow running buffer (1X PBS, 0.05% Tween-20). A Biotin-FAM detecting lateral flow strip (HybriDetect, Milenia Biotec) is then placed into the reaction mix to generate a readout.

Extended crRNA Configuration:

For the extended crRNA configuration, crRNA with a spacer sequence targeting the DNA amplicon of interest was synthesized with an extended 3′ region (IDT). This was annealed to tracrRNA (IDT) at a concentration of 10 μM of each component in Duplex Buffer (IDT) by heating to 95° C. and allowing it to cool at room temperature until equilibrated. For amplicon capture, 2 μL of FAM-labeled RPA product is mixed with 31 nM dCas9 (IDT) and 33 nM annealed guide RNA, and 666 nM biotinylated capture probe in 1X NEBuffer r3.1 (NEB) in a 30 μL reaction mix. No incubation time is required before the addition of 70 μL lateral flow running buffer (1X PBS, 0.05% Tween-20). A Biotin-FAM detecting lateral flow strip (HybriDetect, Milenia Biotec) is then placed into the reaction mix to generate a readout.

Extended sgRNA Configuration:

For the extended sgRNA configuration, sgRNA was generated using in-vitro transcription. Single-stranded DNA (ssDNA) templates for this transcription were generated (IDT) and used with a AmpliScribe T7 High Yield Transcription Kit (Biosearch Technologies) to generate RNA following the manufacturer's instructions. The ssDNA template contains T7 promoter upstream (CCCTATAGTGAGTCGTATTAGCGC (SEQ ID NO: 1)) and was supplemented with a reverse complement oligo (GCGCTAATACGACTCACTATAGGG (SEQ ID NO: 2)) for priming of the transcription. The RNA was purified using a Monarch RNA Cleanup Kit (NEB), and final concentration was determined using a NanoDrop One (Thermo Fisher). For amplicon capture, 20 μL of FAM-labeled RPA product is mixed with 31 nM dCas9 (IDT) 198 nM annealed sgRNA, and 666 nM of biotinylated capture DNA in 1×NEBuffer r3.1 (NEB) in a 30 μL reaction mix. No incubation time is required before the addition of 70 μL lateral flow running buffer (1X PBS, 0.05% Tween-20). A Biotin-FAM detecting lateral flow strip (HybriDetect, Milenia Biotec) is then placed into the reaction mix to generate a readout.

Detection of Human mRNA Isoforms:

This strategy has been successfully used for detection of human mRNA isoforms using RT-RPA amplification and lateral flow assay using two potential guide RNA extension sites for dCas9. The biotinylated split guide RNA (tracer+biotin-crRNA) approach can be used to recruit a FAM bearing RT-RPA amplification product to the streptavidin test line. RT-RPA primers with 5′ FAM modification were designed against the desired exon-exon junction and used to generate FAM-labeled RPA products in the presence of the corresponding mRNA isoform. Following RT-RPA at 42 C for 5-45 min, the amplification product was mixed with the dCas9 cocktail as described above (dCas9 and gRNA) and ran against a lateral flow assay. The lateral flow strip contains FAM targeting gold particles and hence provides for visual detection within 1-2 min. This assay format was capable of detecting concentrations as low as 50 fM of target RNA from a complex RNA mixture (1 ug/μL total RNA). Using 3′ extended sgRNA we were also able to demonstrate sequence specific DNA amplicon capture following a similar RT-RPA sample preparation.

Specific elements of any of the disclosed embodiments can be combined or substituted for elements in other embodiments. Furthermore, while advantages associated with certain embodiments of the disclosure have been described in the context of these embodiments, other embodiments can also exhibit such advantages, and not all embodiments need necessarily exhibit such advantages to fall within the scope of the disclosure.

It should be understood that this disclosure is not limited to the particular methodology, protocols, and reagents, etc., provided herein and as such may vary. The terminology used herein is for the purpose of describing particular embodiments only, and is not intended to limit the scope of the present disclosure, which is defined solely by the claims. The invention is further illustrated by the following example, which should not be construed as further limiting.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A method for detecting a target nucleic acid, the method comprising:
a. Contacting a double-stranded or single-stranded amplicon from amplification of a target nucleic acid with a first probe to form a complex comprising the first probe and the amplicon, wherein the first probe comprises a first nucleic acid strand and a molecule bound with the first nucleic acid strand, wherein the molecule is capable of localizing a single-stranded nucleic acid strand to a double-stranded nucleic acid or enhancing the kinetics of hybridization between two single-stranded nucleic acids, and wherein the first nucleic acid strand comprises a binding domain comprising a nucleotide sequence substantially complementary to at least a first portion of the amplicon; and
b. detecting the complex comprising the first probe and the amplicon from step (a).

2. The method of claim 1, wherein the molecule capable of localizing a single-stranded nucleic acid strand to a double-stranded nucleic acid or enhancing the kinetics of hybridization between two single-stranded nucleic acids is selected from the group consisting of recombinases, CRISPR-Cas proteins, single-stranded binding proteins, site-specific recombinases, zinc finger nucleases, transcription activator-like effector nucleases (TALEN), transcription factors, and any combinations thereof.

3. The method of claim 1, wherein the molecule capable of localizing a single-stranded nucleic acid strand to a double-stranded nucleic acid or enhancing the kinetics of hybridization between two single-stranded nucleic acids is a recombinase or CRISPR-Cas protein, optionally, the molecule capable of localizing a single-stranded nucleic acid strand to a double-stranded nucleic acid lacks nuclease activity.

4. The method of claim 1, wherein one of the amplicon and the first nucleic acid strand comprises a reporter molecule capable of producing a detectable signal and the other of the amplicon and the first nucleic acid strand comprises a capture ligand, and wherein said step of detecting the complex comprises detecting the reporter molecule in the complex.

5. The method of claim 4, wherein the first nucleic acid strand comprises a first hybridization domain linked with the binding domain and the first probe comprises a second nucleic acid strand hybridized with the first hybridization domain of the first nucleic acid strand.

6. The method of claim 5, wherein the first nucleic acid strand comprises a first hybridization domain linked with the binding domain linked with a second hybridization domain, and the first probe further comprises a second nucleic acid strand hybridized with the first nucleic acid strand,
wherein the second nucleic acid strand comprises a first hybridization domain linked with a non-hybridizing domain linked with a second hybridization domain,
wherein the first hybridization domain of the first nucleic acid strand and the second hybridization domain of the second nucleic acid strand are hybridized with each other forming a first double-stranded region,
wherein the second hybridization domain of the first nucleic acid strand and the first hybridization domain of the second nucleic acid strand are hybridized with each other forming a second double-stranded region,
wherein the binding domain of the first nucleic acid strand and the non-hybridizing domain of the second nucleic acid strand do not hybridize to each other, and
wherein one of the amplicon and the first nucleic acid strand comprises a reporter molecule capable of producing a detectable signal and the other of the amplicon and the first nucleic acid strand comprises a capture ligand, and
wherein said step of detecting the complex comprises detecting the reporter molecule in the complex.

7. The method of claim 1, wherein the first nucleic acid strand comprises the reporter molecule and the amplicon comprises the capture ligand.

8. The method of claim 1, wherein the amplicon comprises the reporter molecule and the first nucleic acid strand comprises the capture ligand.

9. The method of claim 8, wherein the amplicon further comprises a second reporter molecule.

10. The method of claim 1, wherein the first probe comprises a second nucleic acid strand hybridized with the first strand and forming a double-stranded structure comprising a single-stranded loop region,
wherein the first nucleic acid strand comprises a first hybridization domain linked to the binding domain linked to a second hybridization domain,
wherein the second nucleic acid strand comprises a first hybridization domain linked to a non-hybridizing domain linked to a second hybridization domain,
wherein the first hybridization domain of the first nucleic acid strand and the second hybridization domain of the second nucleic acid strand are hybridized with each other forming a first double-stranded region,
wherein the second hybridization domain of the first nucleic acid strand and the first hybridization domain of the second nucleic acid strand are hybridized with each other forming a second double-stranded region,
wherein the binding domain of the first nucleic acid strand and the non-hybridizing domain of the second nucleic acid strand do not hybridize to each other,
wherein the first nucleic acid strand comprises a reporter molecule capable of producing a detectable signal and the second nucleic acid strand comprises a capture ligand, and
where absence of a detectable signal from the reporter molecule indicates presence of the target nucleic acid.

11. The method of claim 1, wherein method comprises contacting the amplicon with the first probe and a second probe to form a complex comprising the first probe, the second probe and the amplicon, and
wherein the second probe comprises a first nucleic acid strand (first nucleic acid of the second probe) and a molecule capable of localizing a single-stranded nucleic acid strand to a double-stranded nucleic acid bound with the first nucleic acid strand of the second probe, and wherein the first nucleic acid strand of the second probes comprises a binding domain comprising a nucleotide sequence substantially complementary to at least a second portion of the amplicon,
wherein one of the first nucleic acid strand of the first probe and the first nucleic acid strand of the second probe comprises a reporter molecule capable of producing a detectable signal and the other of the first nucleic acid strand of the first probe and the first nucleic acid strand of the second probe comprises a capture ligand, and
wherein said step of detecting the complex comprises detecting the reporter molecule in the complex.

12. The method of claim 1, wherein detecting the complex comprises detecting a detectable signal produced by the reporter molecule.

13. The method of 1, wherein said step of detecting the complex comprises fluorescence detection, luminescence detection, chemiluminescence detection, colorimetric detection, immunofluorescence detection, or electrochemical detection.

14. The method of claim 1, wherein said step of detecting the complex comprises a lateral flow assay.

15. The method of claim 14, wherein said step of detecting the complex comprises:
a. contacting the complex with a lateral flow device, wherein the lateral flow device or the micro-array plate comprises a capture/test region comprising a capture probe immobilized thereon, wherein the capture probe is capable of binding with capture label; and
b. detecting the reporter molecule in the complex captured by the capture probe.

16. The method of claim 1, wherein said step of detecting the complex comprises micro-array detection.

17. The method of claim 16, wherein said step of detecting the complex comprises:
a. contacting the complex with a micro-array plate, wherein the micro-array plate comprises a capture/test region comprising a capture probe immobilized thereon and capable of binding with capture label; and
b. detecting the reporter molecule in the complex captured by the capture probe.

18. The method of claim 1, wherein the first nucleic acid strand of the first probe comprises a reporter molecule capable of producing a detectable signal, and wherein said step of detecting the complex comprises:
a. contacting the complex with a lateral flow device or a micro-array plate, wherein the lateral flow device or the micro-array plate comprises a capture/test region comprising a capture nucleic acid strand immobilized thereon, wherein the capture nucleic acid strand is bound with a molecule capable of localizing a single-stranded nucleic acid strand to a double-stranded nucleic acid and comprises a nucleotide sequence substantially complementary to at least a second portion of the amplicon; and
b. detecting the reporter molecule in the complex captured by the capture probe.

19. The method of claim 18, wherein the method comprises immobilizing the capture nucleic acid strand on the capture/test region prior to contacting with the complex.

20. The method of claim 18, wherein the capture nucleic acid comprises a capture ligand conjugated thereto and the capture/test region comprises a capture probe immobilized thereon, and wherein the capture probe is capable of binding with capture ligand of the capture nucleic acid strand, optionally, the capture nucleic acid is complexed with a molecule capable of localizing a single-stranded nucleic acid strand to a double-stranded nucleic acid.

21. The method of claim 1, wherein the first nucleic acid strand comprises a first hybridization domain linked to the binding domain, and the first probe comprises a second nucleic acid strand hybridized with the hybridization domain of the first strand, wherein one of the first and second nucleic strand comprises a reporter molecule capable of producing a detectable signal and the other of the first and second nucleic strand comprises a quencher molecule, and wherein the quencher molecule quenches the detectable signal from the reporter molecule when the first and second nucleic acid strands are hybridized with each other, wherein the first hybridization domain and the binding domain together comprise a nucleotide sequence substantially complementary to at least a portion of the amplicon, and wherein said step of detecting the complex comprises detecting a detectable signal produced by the reporter molecule.

22. The method of claim 1, wherein the first nucleic acid strand comprises a first hybridization domain linked with the binding domain linked with a second hybridization domain, and the first probe comprises a second nucleic acid strand hybridized with the first nucleic acid strand,
wherein the second nucleic acid strand comprises a first hybridization domain linked with a non-hybridizing domain linked with a second hybridization domain,
wherein the first hybridization domain of the first nucleic acid strand and the second hybridization domain of the second nucleic acid strand are hybridized with each other forming a first double-stranded region,
wherein the second hybridization domain of the first nucleic acid strand and the first hybridization domain of the second nucleic acid strand are hybridized with each other forming a second double-stranded region,
wherein the binding domain of the first nucleic acid strand and the non-hybridizing domain of the second nucleic acid strand do not hybridize to each other,
wherein one of the first and second nucleic strand comprises a reporter molecule capable of producing a detectable signal and the other of the first and second nucleic strand comprises a quencher molecule, and wherein the quencher molecule quenches the detectable signal from the reporter molecule when the first and second nucleic acid strands are hybridized with each other, and
wherein said step of detecting the complex comprises detecting a detectable signal produced by the reporter molecule.

23. The method of claim 1, wherein said detecting the complex comprises fluorescence detection, luminescence detection, chemiluminescence detection, colorimetric detection, immunofluorescence detection, or electrochemical detection.

24. The method of claim 1, wherein the molecule capable of localizing a single-stranded nucleic acid strand to a double-stranded nucleic acid is a recombinase.

25. The method of claim 24, wherein the recombinase is selected from the group consisting of RecA, UvsX, RadA, Rad51, Dmcl, UvsY, Cre, Flp, Dre, SCre, VCre, Vika, B2, B3, KD, ΦC31, Bxb1, λ, HK022, HP1, γδ, ParA, Tn3, Gin, R4, TP901-1, TG1, PhiRv1, PhiBT1, SprA, XisF, TnpX, R, A118, spoIVCA, PhiMR11, SCCmec, TndX, XerC, XerD, XisA, Hin, Cin, mrpA, beta, PhiFC1, Fre, Clp, sTre, FimE, HbiFm, and homologues thereof, and modified versions thereof.

26. The method of claim 1, wherein the molecule capable of localizing a single-stranded nucleic acid strand to a double-stranded nucleic acid is a CRISPR-Cas protein selected from the group consisting of Cas9 (also known as Csn1 and Csx12), C2c1, C2c3, Casl, Cas100, Cas12a, Cas12b, Cas12c, Cas12d, Cas12e, Cas13a, Cas13b, Cas13c, Cas1B, Cas2, Cas3, Cas4, Cas5, Cas6, Cas7, Cas8, Casl, CaslB, CaslO, Cmr1, Cmr3, Cmr4, Cmr5, Cmr6, Cpf1, Csa5, Csa5, CsaX, Csb1, Csb2, Csb3, Csc1, Csc2, Cse1, Cse2, Csf1, Csf2, Csf3, Csf4, Csm2, Csm3, Csm4, Csm5, Csm6, Csn2, Csx1, Csx10, Csx14, Csx15, Csx16, Csx17, Csx3, Csy1, Csy2, Csy3, and homologues thereof, or modified versions thereof, optionally, the CRISPR-Cas protein lack nuclease activity.

27. The method of claim 1, wherein the reporter molecule is selected from the group consisting of fluorescent molecules, radioisotopes, chromophores, enzymes, enzyme substrates, chemiluminescent moieties, bioluminescent moieties, echogenic substances, non-metallic isotopes, optical reporters, paramagnetic metal ions, ferromagnetic metals, quantum dots (or semiconductor nanocrystals), nanoparticles (e.g. gold nanoparticles used in lateral flow assays, carbon nanoparticles), and latex and fluorescent beads.

28. The method of claim 1, wherein the capture ligand is biotin or a nucleic acid, optionally, the nucleic acid and comprises a toehold domain.

29. A composition comprising:
a. a probe and a double-stranded or single-stranded amplicon from amplification of a target nucleic acid, wherein the first probe comprises a first nucleic acid strand and a molecule bound with the first nucleic acid strand, wherein the molecule is capable of localizing a single-stranded nucleic acid strand to a double-stranded nucleic acid or enhancing the kinetics of hybridization between two single-stranded nucleic acids, and wherein the first nucleic acid strand comprises a binding domain comprising a nucleotide sequence substantially complementary to at least a first portion of the amplicon; or
b. a probe and a double-stranded or single stranded amplicon from amplification of a target nucleic acid, wherein the probe comprises a first nucleic acid strand, a second nucleic acid strand, and a molecule capable of localizing a single-stranded nucleic acid strand to a double-stranded nucleic acid or enhancing the kinetics of hybridization between two single-stranded nucleic acids, wherein the first nucleic acid strand comprises a binding domain comprising a nucleotide sequence substantially complementary to at least a first portion of the amplicon, wherein the first nucleic acid strand comprises a first hybridization domain linked with the binding domain and the second nucleic acid strand is hybridized with the first hybridization domain of the first nucleic acid strand, and wherein the first nucleic acid strand comprises a reporter molecule capable of producing a detectable signal and the second nucleic acid strand comprises a capture ligand; or
c. a first probe, a second probe, and a double-stranded or single-stranded amplicon from amplification of a target nucleic acid, wherein the first probe comprises a first nucleic acid strand and a first molecule capable of localizing a single-stranded nucleic acid strand to a double-stranded nucleic acid or enhancing the kinetics of hybridization between two single-stranded nucleic acids, and wherein the first nucleic acid strand of the first probe comprises a binding domain comprising a nucleotide sequence substantially complementary to at least a first portion of the amplicon,
wherein the second probe comprises a first nucleic acid strand and a first molecule capable of localizing a single-stranded nucleic acid strand to a double-stranded nucleic acid or enhancing the kinetics of hybridization between two single-stranded nucleic acids, and wherein the first nucleic acid strand of the second probe comprises a binding domain comprising a nucleotide sequence substantially complementary to at least a second portion of the amplicon, and
wherein one of the first nucleic acid strand of the first probe and the first nucleic acid strand of the second probe comprises a reporter molecule capable of producing a detectable signal and the other of the first nucleic acid strand of the first probe and the first nucleic acid strand of the second probe comprises a capture ligand; or
d. a probe and a double-stranded or single-stranded amplicon from amplification of a target nucleic acid, wherein the probe comprises a first nucleic acid strand, a second nucleic acid strand, and a first molecule capable of localizing a single-stranded nucleic acid strand to a double-stranded nucleic acid or enhancing the kinetics of hybridization between two single-stranded nucleic acids, and wherein the first nucleic acid strand of the first probe comprises a binding domain comprising a nucleotide sequence substantially complementary to at least a first portion of the amplicon, wherein the first nucleic acid strand comprises a first hybridization domain linked with the binding domain and the second nucleic acid strand is hybridized with the first hybridization domain of the first nucleic acid strand,
wherein one of the first and second nucleic strand comprises a reporter molecule capable of producing a detectable signal and the other of the first and second nucleic strand comprises a quencher molecule, and wherein the quencher molecule quenches the detectable signal from the reporter molecule when the first and second nucleic acid strands are hybridized with each other,
wherein the first hybridization domain and the binding domain of the first nucleic acid strand together comprise a nucleotide sequence substantially complementary to at least a portion of the amplicon.
Kits

30. A kit comprising:
a. a primer set for preparing a double-stranded or single-stranded amplicon from a target nucleic acid, and a first probe,
wherein the first probe comprises a first nucleic acid strand and a molecule bound with the first nucleic acid strand, wherein the capable of localizing a single-stranded nucleic acid strand to a double-stranded nucleic acid or enhancing the kinetics of hybridization between two single-stranded nucleic acids,
wherein the first nucleic acid strand comprises a binding domain comprising a nucleotide sequence substantially complementary to at least a first portion of the amplicon prepared from a target nucleic acid using the primer set,
wherein the first nucleic acid strand comprises a reporter molecule capable of producing a detectable signal and at least one primer in the primer set comprises a capture ligand, or the first nucleic acid strand comprises capture ligand and at least one primer in the primer set comprises a reporter molecule capable of producing a detectable signal.
b. a primer set for preparing a double-stranded or single-stranded amplicon from a target nucleic acid, and a first probe,
wherein the first probe comprises a first nucleic acid strand, a second nucleic acid strand, and a molecule capable of localizing a single-stranded nucleic acid strand to a double-stranded nucleic acid or enhancing the kinetics of hybridization between two single-stranded nucleic acids,
wherein the first nucleic acid strand comprises a first hybridization domain linked with a binding domain,
wherein the second nucleic acid strand is hybridized with the first hybridization domain of the first nucleic acid strand,
wherein one of the first and second nucleic strand comprises a reporter molecule capable of producing a detectable signal and the other of the first and second nucleic strand comprises a quencher molecule, and wherein the quencher molecule quenches the detectable signal from the reporter molecule when the first and second nucleic acid strands are hybridized with each other.


SEQUENCE LISTING (2 sequences)

SEQ ID NO: 1 - DNA, 24 bp, synthetic construct
  source 1..24: mol_type = other DNA
ccctatagtgagtcgtattagcgc

SEQ ID NO: 2 - DNA, 24 bp, synthetic construct
  source 1..24: mol_type = other DNA
gcgctaatacgactcactataggg